(19) United States
(12) Patent Application Publication
(10) Pub. No.: US 2023/0181104 A1
(43) Pub. Date: Jun. 15, 2023

(54) SNORE DETECTION AND RESPONSE FOR ADJUSTABLE BED SYSTEMS

(71) Applicant: Sleep Number Corporation, Minneapolis, MN (US)

(72) Inventors: Robert Dean Johnston, Lakeville, MN (US); Julianne Clemens, Bloomington, MN (US); Cory Lee Grabinger, Maple Grove, MN (US); Gary N. Garcia Molina, Madison, WI (US)

(21) Appl. No.: 18/075,913

(22) Filed: Dec. 6, 2022

Related U.S. Application Data
(60) Provisional application No. 63/299,167, filed on Jan. 13, 2022.
(60) Provisional application No. 63/288,171, filed on Dec. 10, 2021.

(51) Int. Cl. A61B 5/00; A47C 19/04; A47C 20/04; A61B 7/00

(52) U.S. Cl. CPC A61B 5/4818; A47C 19/045; A47C 20/04; A61B 5/6892; A61B 7/003; A61B 5/4836; A61B 2562/0247

(57) ABSTRACT

Disclosed herein are systems and methods for detecting snore and automatically adjusting a bed system to a position intended to mitigate the snore. The bed system can include a mattress, an adjustable foundation, and a controller having a processor. The processor can be configured to generate, in response to sensing snoring from a user of the bed system, instructions for tilting the adjustable foundation to a predetermined position such that an angle between a head portion of the adjustable foundation and the ground is greater than an angle between a foot portion of the adjustable foundation and the ground. The ground can be a horizontal plane. The predetermined position can be a position intended to mitigate snore.

### INCORPORATION BY REFERENCE

This application claims priority to U.S. Provisional Application Ser. No. 63/299,167, filed on Jan. 13, 2022, and U.S. Provisional Application Ser. No. 63/288,171, filed on Dec. 10, 2021, the disclosures of which are incorporated by reference in their entirety.

### TECHNICAL FIELD

The present document relates to detection of snore and automatic bed adjustment.

### BACKGROUND

In general, a bed is a piece of furniture used as a location to sleep or relax. Many modern beds include a soft mattress on a bed frame. The mattress may include springs, foam material, and/or an air chamber to support the weight of one or more occupants.

### SUMMARY

This document generally relates to detecting that a user of a bed system is snoring and automatically adjusting the bed system to reduce or mitigate the snoring. More particularly, the disclosed techniques provide for detecting that the user is snoring based on a combination of acoustic and pressure signals. Using machine learning techniques, the acoustic and pressure signals can be correlated to determine whether the user is in fact snoring and which side of the bed system from which the snoring originates. Based on these determinations, the disclosed techniques also provide for automated adjustment of the bed system to a position that can reduce, stop, mitigate, or otherwise prevent the snoring. The position, as an illustrative example, can be a reverse Trendelenburg position, in which an adjustable foundation of the bed system is inclined such that a head of the user is elevated above feet of the user at a predetermined angle (e.g., a 7° angle relative to the ground). The disclosed techniques can provide for reducing or eliminating the user's snore without having to wake up or disturb the sleep of the user and/or a partner user of the bed system.

Some embodiments described herein include a bed system having a mattress, an adjustable foundation, and a controller having a processor. The processor can be configured to generate, in response to sensing snoring from a user of the bed system, instructions for tilting the adjustable foundation to a predetermined position such that a head portion of the adjustable foundation can be higher than a foot portion of the adjustable foundation.

Embodiments described herein can include one or more optional features. For example, the bed system can also include an acoustic sensor for sensing audio at the bed system and the processor can be configured to: receive acoustic signals from the acoustic sensor, and analyze the acoustic signals to determine that the user is snoring.

The bed system can also include a pressure sensor for sensing pressure on the mattress, and the processor can: receive pressure signals from the pressure sensor, and analyze the pressure signals to determine that the user is snoring. The processor can also analyze the pressure signals to determine a left side or a right side of the mattress from which the snoring originates.

In some embodiments, the bed system can also include a group of pressure sensors. Each of the pressure sensors can be positioned in a support leg of the adjustable foundation and the pressure sensors can be configured to sense pressure on the bed system that can be indicative of a location of the user on the mattress.

The bed system, in some embodiments, can also include at least one sensor, and the processor can further be configured to: receive signals from the at least one sensor, and analyze the signals to determine that the user is snoring. In some implementations, the at least one sensor can be a pressure sensor. In some implementations, the at least one sensor can be an acoustic sensor. The signals can include at least one of acoustic signals and pressure signals. In some implementations, the signals can include acoustic signals and pressure signals, and the processor can correlate the acoustic signals with the pressure signals to determine (i) whether the user is snoring and (ii) a left side or right side of the mattress from which the snoring originates.

In some embodiments, the bed system can also include an articulation system that can tilt the adjustable foundation to the predetermined position. Moreover, the processor can transmit, in response to determining that the user is snoring, instructions to the articulation system to tilt the adjustable foundation to the predetermined position, and the articulation system can: raise, in response to receiving the instructions from the processor, legs supporting the head portion of the adjustable foundation to a height that corresponds to the angle between the head portion of the adjustable foundation and the ground when the adjustable foundation is in the predetermined position.

Moreover, in some embodiments, the articulation system can lower, in response to receiving the instructions from the processor, legs supporting the foot portion of the adjustable foundation to a height that corresponds to the angle between the foot portion of the adjustable foundation and the ground when the adjustable foundation is in the predetermined position. In some embodiments, the angle between the foot portion of the adjustable foundation and the ground can be less than 7°.

In some embodiments, the processor can transmit, in response to determining that the user has woken up, instructions to the articulation system to move the adjustable foundation into a neutral position, and the articulation system can lower, in response to receiving the instructions from the processor, the legs supporting the head portion of the adjustable foundation to a second height that corresponds to the neutral position. In the neutral position, the adjustable foundation can be parallel with the ground. The articulation system can also raise, in response to receiving the instructions from the processor, the legs supporting the foot portion of the adjustable foundation to the second height that corresponds to the neutral position.

In some embodiments, the articulation system can adjust, based on receiving an indication of user input from a user device indicating selection of an option to move the adjustable foundation to another position, a height of one or more of the legs supporting the adjustable foundation to move the adjustable foundation to the another position.

Some embodiments described herein include a bed system having: a mattress, an adjustable foundation, an acoustic sensor for sensing audio proximate the mattress, a pressure sensor for sensing pressure on the mattress, and a controller comprising a processor. The processor can: analyze acoustic signals obtained from the acoustic sensor to determine one or more audio signals for a user on the mattress, analyze pressure signals obtained from the pressure sensor to determine one or more biometric parameters for the user on the mattress, correlate the acoustic signals with the pressure signals to determine (i) whether the user is snoring and (ii) a left side or a right side of the mattress from which the snoring originates, and adjust a left side or a right side of the adjustable foundation that corresponds to the left side or the right side of the mattress from which the snoring originates by actuating at least one motor that is configured to tilt the adjustable foundation such that a head portion of the adjustable foundation is higher than a foot portion of the adjustable foundation on the adjusted left side or the adjusted right side of the adjustable foundation.

Embodiments described herein can include one or more optional features. For example, adjusting the left side or the right side of the adjustable foundation can include actuating the at least one motor to incline the left side or the right side of the adjustable foundation by 7° relative to the ground, where the ground can be a horizontal plane.

In some embodiments, the left side of the adjustable foundation can support a first user and the right side of the adjustable foundation supports a second user when the first and second users are resting on the respective left and right sides of the mattress.

In some embodiments, the adjustable foundation can remain inclined until the controller receives an indication of user input to move the adjustable foundation to a predetermined position. The processor can be further configured to adjust the left side or the right side of the adjustable foundation to the predetermined position by actuating the at least one motor to lower, based on the indication of user input, the left side or the right side of the adjustable foundation to the predetermined position. The predetermined position can be a flat position.

In some embodiments, the processor can correlate the acoustic signals with the pressure signals using a machine learning model that was previously trained to detect snore. The machine learning model could have been trained using a dataset of training acoustic data that was annotated with pressure signals when the adjustable foundation was inclined at one or more different angles.

Moreover, the processor can determine whether the user on the mattress is snoring based on sound waves generated by the user and detected by the pressure sensor.

In some embodiments, the mattress can be an inflatable air mattress. The bed system can also include a pump that can be operably connected to the inflatable air mattress. The pressure sensor can include a pressure transducer positioned in the pump and fluidically connected to both a manifold of the pump and to the inflatable air mattress.

In some embodiments, the bed system can include a first air chamber, a first pressure sensor for sensing air pressure of the first air chamber, a second air chamber, and a second pressure sensor for sensing air pressure of the second air chamber. The processor can: determine biometric parameters for a first user supported by the first air chamber based on air pressure sensed by the first pressure sensor, detect biometric parameters for a second user supported by the second air chamber based on air pressure sensed by the second pressure sensor, determine, based on the biometric parameters determined for the first and second users, that one of the first and second users is snoring, and adjust, based on determining which of the first and second users is snoring, a side of the adjustable foundation supporting one of the first and second air chambers by actuating the at least one motor that can be configured to incline the adjustable foundation.

In some embodiments, the bed system can also include an articulation system having the at least one motor for articulating the left side of the adjustable foundation, a second motor for articulating the right side of the adjustable foundation, and one or more controllers for controlling movement of the motors. In some embodiments, the adjustable foundation can include a plurality of legs, and one or more pressure sensors can be coupled to the plurality of legs, the one or more pressure sensors being configured to sense force applied to the plurality of legs when the user is resting on the mattress.

Some embodiments described herein include a bed system having a mattress, an adjustable foundation, and a controller including a processor. The processor can generate, in response to sensing snoring from a user of the bed system, instructions for tilting the adjustable foundation to a predetermined position such that a head portion of the adjustable foundation and a foot portion of the adjustable foundation can be in-line with respect to each-other and tilted with respect to horizontal.

Some embodiments described herein can include a bed system having a mattress, an adjustable foundation, and a controller including a processor, the processor being configured to generate, in response to sensing snoring from a user of the bed system, instructions for tilting a head portion of the adjustable foundation and a foot portion of the adjustable foundation at substantially a same angle.

Some embodiments described herein can also include a bed system having a mattress, an adjustable foundation, and a controller including a processor, the processor being configured to generate, in response to sensing snoring from a user of the bed system, instructions for tilting the adjustable foundation such that a head portion of the adjustable foundation can be in-line with and higher than a foot portion of the adjustable foundation.

Some embodiments described herein may also include a bed system having a mattress, an adjustable foundation, and a controller including a processor. The processor can generate, in response to sensing snoring from a user of the bed system, instructions for adjusting the adjustable foundation such that a head portion of the adjustable foundation can be inclined above a foot portion of the adjustable foundation, the adjustable foundation remaining in a substantially straight position when inclined.

Embodiments described herein can optionally include one or more of the following features. For example, in the predetermined position: the head portion of the adjustable foundation can be tilted above the foot portion of the adjustable foundation, and the adjustable foundation can remain inclined along a substantially straight plane. In some implementations, in the predetermined position, the adjustable foundation can be maintained along an inclined and substantially straight plane. Moreover, adjusting the left side or the right side of the adjustable foundation can include actuating the at least one motor that can tilt the adjustable foundation such that the head portion of the adjustable foundation can be higher than the foot portion of the adjustable foundation and can be maintained along an inclined and substantially straight plane on the adjusted left side or the adjusted right side of the adjustable foundation.

The devices, system, and techniques described herein may provide one or more of the following advantages. For example, tilting an entire adjustable foundation can maintain the user asleep and the user's comfort during sleep while also mitigating or preventing the user from snoring. Rather than tilting just the user's head when snore is detected, the disclosed techniques provide for tilting the user's entire body such that the head is elevated slightly above the user's feet while the body is maintained in a plane. This position can advantageously mitigate the user's snoring while also ensuring comfortability of the user by maintaining the user's back and neck in an aligned position. For example, if the user sleeps on their side or stomach, tilting the entire adjustable foundation can ensure that the user's head and/or back remain aligned and not put into uncomfortable positions that can cause the user to wake up or experience soreness in the back and/or neck when the user wakes up.

Similarly, the adjustable foundation may be quietly tilted (e.g., inclined) to a position that mitigates or prevents snore so that the user is neither disturbed by sound of actuators adjusting the foundation to the position nor an angle of the position. As a result, the user can maintain quality sleep and the user's sleep (and/or a partner's sleep) may not be disturbed.

As another example, snore can be accurately detected based on correlating acoustic and pressure signals. Correlating the acoustic signals with the pressure signals can be beneficial to determine which user in the bed is snoring such that their side of the bed can be adjusted to mitigate their snoring. As mentioned above, the user's side of the bed can be tilted into a position for snore mitigation (e.g., in which the user's head is tilted/inclined above the user's feet while the bed remains substantially straight) without disrupting sleep of either user.

Moreover, once the adjustable foundation is tilted into the position for snore mitigation, the foundation can remain in that position until the user wakes up. This can be advantageous to ensure safety of the user, other users, and/or pets/animals. For example, if the adjustable foundation moves from the position for snore mitigation to another position (such as a flat position) while the user is still sleeping, the user may get their hand caught in mechanisms of the adjustable foundation, which can cause injury. The disclosed techniques, on the other hand, provide for the adjustable foundation to remain in the tilted position to avoid causing injury to the user while the user is resting in the bed.

As another example, using machine learning techniques can provide for more accurate snore detection and mitigation. Machine learning techniques can make snore detection both faster and/or accurate such that the snore can be mitigated with minimal to no disturbance of the user or another user of the bed. The machine learning techniques can also provide for noisy and complex sensor data, such as acoustic and pressure signals, to be quickly and efficiently converted into accurate snore detection information.

As another example, the disclosed techniques can provide for detecting various health conditions of the users of the bed system. For example, moving the bed system into a position for snore mitigation can be beneficial to detect instances of sleep apnea. After all, apnea-related patterns manifest in spectral content of an audio signal. The nature of these patterns depends on a user's position on the bed. For instance, the spectral content of a snoring audio signal in an apnea patient has higher energy for frequency above 800 Hz when the user is in one position (e.g., a position for snore mitigation) than when the user is in another position in which the spectral content difference is not as visible.

The details of one or more implementations are set forth in the accompanying drawings and the description below. Other features, aspects and potential advantages will be apparent from the accompanying description and figures.

### DESCRIPTION OF DRAWINGS

FIG. 1 shows an example air bed system.

FIG. 2 is a block diagram of an example of various components of an air bed system.

FIG. 3 shows an example environment including a bed in communication with devices located in and around a home.

FIGS. 4A and 4B are block diagrams of example data processing systems that can be associated with a bed.

FIGS. 5 and 6 are block diagrams of examples of motherboards that can be used in a data processing system associated with a bed.

FIG. 7 is a block diagram of an example of a daughterboard that can be used in a data processing system associated with a bed.

FIG. 8 is a block diagram of an example of a motherboard with no daughterboard that can be used in a data processing system associated with a bed.

FIG. 9 is a block diagram of an example of a sensory array that can be used in a data processing system associated with a bed.

FIG. 10 is a block diagram of an example of a control array that can be used in a data processing system associated with a bed

FIG. 11 is a block diagram of an example of a computing device that can be used in a data processing system associated with a bed.

FIGS. 12-16 are block diagrams of example cloud services that can be used in a data processing system associated with a bed.

FIG. 17 is a block diagram of an example of using a data processing system that can be associated with a bed to automate peripherals around the bed.

FIG. 18 is a schematic diagram that shows an example of a computing device and a mobile computing device.

FIG. 19 is a conceptual diagram of a bed system environment for detecting and responding to snore.

FIGS. 20A-E show an example bed system inclined to various positions for mitigating detected snore.

FIG. 21 shows an example bed system in which one side of the bed system is inclined to a position for mitigating detected snore.

FIG. 22 is a swimlane diagram of an example process for detecting and responding to snore.

FIG. 23 is a flowchart of a process for training a machine learning model to detect snore.

FIG. 24 is a flowchart of a process for determining that a user is snoring based on pressure data.

FIG. 25 is a flowchart of a process for determining that a user is snoring based on acoustic data.

FIG. 26 is a conceptual diagram of a bed system for detecting and responding to snore.

Like reference symbols in the various drawings indicate like elements.

### DETAILED DESCRIPTION

This document generally relates to a bed system for detecting and responding to snore. More particularly, the disclosed techniques provide for detecting that a user is snoring based on correlating different sensor data, such as acoustic signals and pressure signals. The sensor data can be correlated using machine learning techniques to accurately detect whether the user is snoring and from which side of the bed system the snoring originates. Snoring may become more prevalent with age and can disturb a user of the bed system. Snore detection, as described herein, can be used to automatically and gently raise a head portion of the bed system to temporarily alleviate moderate snoring and improve restful sleep of user(s) of the bed system.

Based on these detections, a controller of the bed system can automatically cause actuators (e.g., motors) of an adjustable foundation of the bed system to incline or tilt the user's side of the bed system to a predetermined position. The predetermined position can be a position for snore mitigation, in which the user's head can be elevated (e.g., inclined or raised) above the user's feet while the body is maintained in an inclined plane and the adjustable foundation is substantially straight. In this position, snore can be mitigated, reduced, or otherwise prevented. Sleeping in this position in which a user's head is elevated can open the user's airways for easy breathing and increased blood flow for improved circulation. An increase in blood oxygen may also reduce stress on the user's heart while maintaining proper spinal alignment, which can improve or otherwise maintain health of the user while they are asleep. Using the disclosed bed system, automated adjustments can further be made to mitigate sleep apnea and acid reflux.

Moreover, adjusting the bed system to this position can be achieved quietly and non-intrusively such that the user's sleep, comfort of the user during sleep, nor a partner's sleep, is disturbed. The user can continue to maintain quality sleep and comfortability when the bed system, or the user's side of the bed, is adjusted to the position for snore mitigation.

Example Airbed Hardware

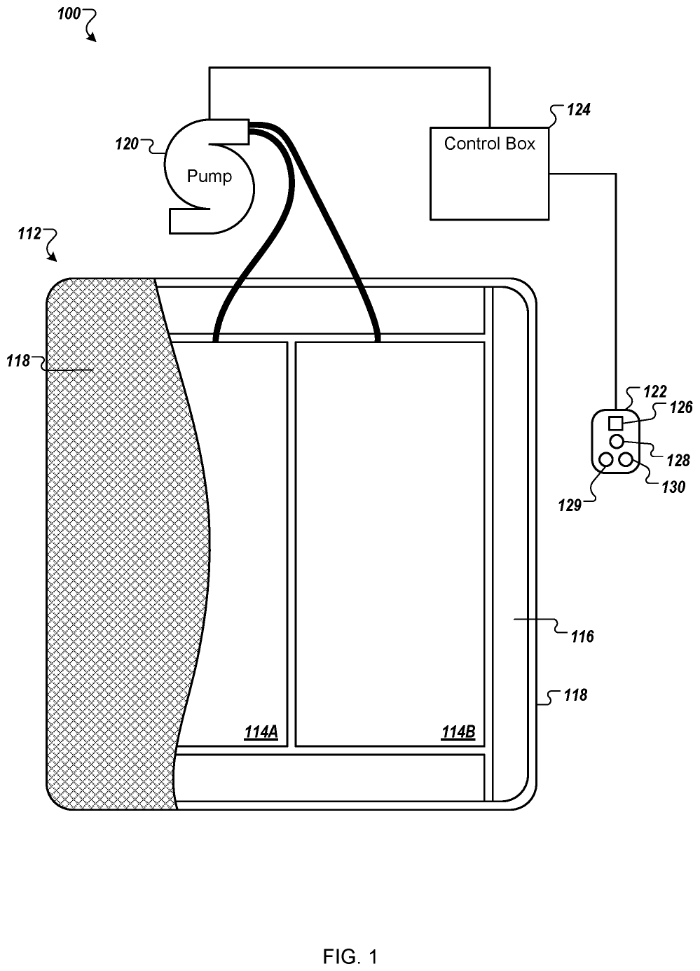

FIG. 1 shows an example air bed system 100 that includes a bed 112. The bed 112 can be a mattress that includes at least one air chamber 114 surrounded by a resilient border 116 and encapsulated by bed ticking 118. The resilient border 116 can comprise any suitable material, such as foam. In some embodiments, the resilient border 116 can combine with a top layer or layers of foam (not shown in FIG. 1) to form an upside down foam tub. In other embodiments, mattress structure can be varied as suitable for the application.

As illustrated in FIG. 1, the bed 112 can be a two chamber design having first and second fluid chambers, such as a first air chamber 114A and a second air chamber 114B. Sometimes, the bed 112 can include chambers for use with fluids other than air that are suitable for the application. For example, the fluids can include liquid. In some embodiments, such as single beds or kids' beds, the bed 112 can include a single air chamber 114A or 114B or multiple air chambers 114A and 114B. Although not depicted, sometimes, the bed 112 can include additional air chambers.

The first and second air chambers 114A and 114B can be in fluid communication with a pump 120. The pump 120 can be in electrical communication with a remote control 122 via control box 124. The control box 124 can include a wired or wireless communications interface for communicating with one or more devices, including the remote control 122. The control box 124 can be configured to operate the pump 120 to cause increases and decreases in the fluid pressure of the first and second air chambers 114A and 114B based upon commands input by a user using the remote control 122. In some implementations, the control box 124 is integrated into a housing of the pump 120. Moreover, sometimes, the pump 120 can be in wireless communication (e.g., via a home network, WIFI, BLUETOOTH, or other wireless network) with a mobile device via the control box 124. The mobile device can include but is not limited to the user's smartphone, cell phone, laptop, tablet, computer, wearable device, home automation device, or other computing device. A mobile application can be presented at the mobile device and provide functionality for the user to control the bed 112 and view information about the bed 112. The user can input commands in the mobile application presented at the mobile device. The inputted commands can be transmitted to the control box 124, which can operate the pump 120 based upon the commands.

The remote control 122 can include a display 126, an output selecting mechanism 128, a pressure increase button 129, and a pressure decrease button 130. The remote control 122 can include one or more additional output selecting mechanisms and/or buttons. The display 126 can present information to the user about settings of the bed 112. For example, the display 126 can present pressure settings of both the first and second air chambers 114A and 114B or one of the first and second air chambers 114A and 114B. Sometimes, the display 126 can be a touch screen, and can receive input from the user indicating one or more commands to control pressure in the first and second air chambers 114A and 114B and/or other settings of the bed 112.

The output selecting mechanism 128 can allow the user to switch air flow generated by the pump 120 between the first and second air chambers 114A and 114B, thus enabling control of multiple air chambers with a single remote control 122 and a single pump 120. For example, the output selecting mechanism 128 can by a physical control (e.g., switch or button) or an input control presented on the display 126. Alternatively, separate remote control units can be provided for each air chamber 114A and 114B and can each include the ability to control multiple air chambers. Pressure increase and decrease buttons 129 and 130 can allow the user to increase or decrease the pressure, respectively, in the air chamber selected with the output selecting mechanism 128. Adjusting the pressure within the selected air chamber can cause a corresponding adjustment to the firmness of the respective air chamber. In some embodiments, the remote control 122 can be omitted or modified as appropriate for an application. For example, as mentioned above, the bed 112 can be controlled by a mobile device in wired or wireless communication with the bed 112.

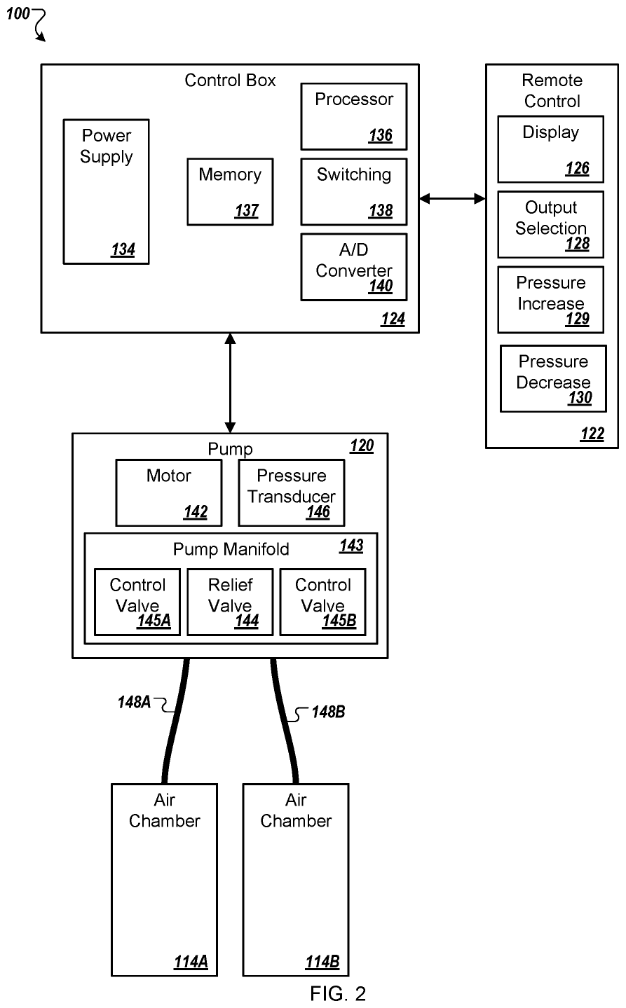

FIG. 2 is a block diagram of an example of various components of an air bed system. For example, these components can be used in the example air bed system 100. As shown in FIG. 2, the control box 124 can include a power supply 134, a processor 136, a memory 137, a switching mechanism 138, and an analog to digital (A/D) converter 140. The switching mechanism 138 can be, for example, a relay or a solid state switch. In some implementations, the switching mechanism 138 can be located in the pump 120 rather than the control box 124.

The pump 120 and the remote control 122 can be in two-way communication with the control box 124. The pump 120 includes a motor 142, a pump manifold 143, a relief valve 144, a first control valve 145A, a second control valve 145B, and a pressure transducer 146. The pump 120 is fluidly connected with the first air chamber 114A and the second air chamber 114B via a first tube 148A and a second tube 148B, respectively. The first and second control valves 145A and 145B can be controlled by switching mechanism 138, and are operable to regulate the flow of fluid between the pump 120 and first and second air chambers 114A and 114B, respectively.

In some implementations, the pump 120 and the control box 124 can be provided and packaged as a single unit. In some implementations, the pump 120 and the control box 124 can be provided as physically separate units. In yet some implementations, the control box 124, the pump 120, or both can be integrated within or otherwise contained within a bed frame, foundation, or bed support structure that supports the bed 112. Sometimes, the control box 124, the pump 120, or both can be located outside of a bed frame, foundation, or bed support structure (as shown in the example in FIG. 1).

The example air bed system 100 depicted in FIG. 2 includes the two air chambers 114A and 114B and the single pump 120 of the bed 112 depicted in FIG. 1. However, other implementations can include an air bed system having two or more air chambers and one or more pumps incorporated into the air bed system to control the air chambers. For example, a separate pump can be associated with each air chamber of the air bed system. As another example, a pump can be associated with multiple chambers of the air bed system. A first pump can, for example, be associated with air chambers that extend longitudinally from a left side to a midpoint of the air bed system 100 and a second pump can be associated with air chambers that extend longitudinally from a right side to the midpoint of the air bed system 100. Separate pumps can allow each air chamber to be inflated or deflated independently and/or simultaneously. Furthermore, additional pressure transducers can be incorporated into the air bed system 100 such that, for example, a separate pressure transducer can be associated with each air chamber.

As an illustrative example, in use, the processor 136 can send a decrease pressure command to one of air chambers 114A or 114B, and the switching mechanism 138 can convert the low voltage command signals sent by the processor 136 to higher operating voltages sufficient to operate the relief valve 144 of the pump 120 and open the respective control valve 145A or 145B. Opening the relief valve 144 can allow air to escape from the air chamber 114A or 114B through the respective air tube 148A or 148B. During deflation, the pressure transducer 146 can send pressure readings to the processor 136 via the A/D converter 140. The A/D converter 140 can receive analog information from pressure transducer 146 and can convert the analog information to digital information useable by the processor 136. The processor 136 can send the digital signal to the remote control 122 to update the display 126 in order to convey the pressure information to the user. The processor 136 can also send the digital signal to one or more other devices in wired or wireless communication with the air bed system, including but not limited to mobile devices such as smartphones, cellphones, tablets, computers, wearable devices, and home automation devices. As a result, the user can view pressure information associated with the air bed system at their mobile device instead of at, or in addition to, the remote control 122.

As another example, the processor 136 can send an increase pressure command. The pump motor 142 can be energized in response to the increase pressure command and send air to the designated one of the air chambers 114A or 114B through the air tube 148A or 148B via electronically operating the corresponding valve 145A or 145B. While air is being delivered to the designated air chamber 114A or 114B in order to increase the firmness of the chamber, the pressure transducer 146 can sense pressure within the pump manifold 143. Again, the pressure transducer 146 can send pressure readings to the processor 136 via the A/D converter 140. The processor 136 can use the information received from the A/D converter 140 to determine the difference between the actual pressure in air chamber 114A or 114B and the desired pressure. The processor 136 can send the digital signal to the remote control 122 to update display 126 in order to convey the pressure information to the user.

Generally speaking, during an inflation or deflation process, the pressure sensed within the pump manifold 143 can provide an approximation of the pressure within the respective air chamber that is in fluid communication with the pump manifold 143. An example method of obtaining a pump manifold pressure reading that is substantially equivalent to the actual pressure within an air chamber includes turning off the pump 120, allowing the pressure within the air chamber 114A or 114B and the pump manifold 143 to equalize, and then sensing the pressure within the pump manifold 143 with the pressure transducer 146. Thus, providing a sufficient amount of time to allow the pressures within the pump manifold 143 and chamber 114A or 114B to equalize can result in pressure readings that are accurate approximations of actual pressure within air chamber 114A or 114B. In some implementations, the pressure of the air chambers 114A and/or 114B can be continuously monitored using multiple pressure sensors (not shown). The pressure sensors can be positioned within the air chambers 114A and/or 114B. The pressure sensors can also be fluidly connected to the air chambers 114A and 114B, such as along the air tubes 148A and 148B.

In some implementations, information collected by the pressure transducer 146 can be analyzed to determine various states of a user laying on the bed 112. For example, the processor 136 can use information collected by the pressure transducer 146 to determine a heartrate or a respiration rate for the user laying on the bed 112. As an illustrative example, the user can be laying on a side of the bed 112 that includes the chamber 114A. The pressure transducer 146 can monitor fluctuations in pressure of the chamber 114A, and this information can be used to determine the user's heartrate and/or respiration rate. As another example, additional processing can be performed using the collected data to determine a sleep state of the user (e.g., awake, light sleep, deep sleep). For example, the processor 136 can determine when the user falls asleep and, while asleep, the various sleep states (e.g., sleep stages) of the user. Based on the determined heartrate, respiration rate, and/or sleep states of the user, the processor 136 can determine information about the user's sleep quality. The processor 136 can, for example, determine how well the user slept during a particular sleep cycle. The processor 136 can also determine user sleep cycle trends. Accordingly, the processor 136 can generate recommendations to improve the user's sleep quality and overall sleep cycle. Information that is determined about the user's sleep cycle (e.g., heartrate, respiration rate, sleep states, sleep quality, recommendations to improve sleep quality, etc.) can be transmitted to the user's mobile device and presented in a mobile application, as described above.

Additional information associated with the user of the air bed system 100 that can be determined using information collected by the pressure transducer 146 includes motion of the user, presence of the user on a surface of the bed 112, weight of the user, heart arrhythmia of the user, snoring of the user or another user on the air bed system, and apnea of the user. One or more other health conditions of the user can also be determined based on the information collected by the pressure transducer 146. Taking user presence detection for example, the pressure transducer 146 can be used to detect the user's presence on the bed 112, e.g., via a gross pressure change determination and/or via one or more of a respiration rate signal, heartrate signal, and/or other biometric signals. Detection of the user's presence on the bed 112 can be beneficial to determine, by the processor 136, one or more adjustments to make to settings of the bed 112 (e.g., adjusting a firmness of the bed 112 when the user is present to a user-preferred firmness setting) and/or peripheral devices (e.g., turning off lights when the user is present, activating a heating or cooling system, etc.).

For example, a simple pressure detection process can identify an increase in pressure as an indication that the user is present on the bed 112. As another example, the processor 136 can determine that the user is present on the bed 112 if the detected pressure increases above a specified threshold (so as to indicate that a person or other object above a certain weight is positioned on the bed 112). As yet another example, the processor 136 can identify an increase in pressure in combination with detected slight, rhythmic fluctuations in pressure as corresponding to the user being present on the bed 112. The presence of rhythmic fluctuations can be identified as being caused by respiration or heart rhythm (or both) of the user. The detection of respiration or a heartbeat can distinguish between the user being present on the bed and another object (e.g., a suitcase, a pet, a pillow, etc.) being placed upon the bed.

In some implementations, fluctuations in pressure can be measured at the pump 120. For example, one or more pressure sensors can be located within one or more internal cavities of the pump 120 to detect fluctuations in pressure within the pump 120. The fluctuations in pressure detected at the pump 120 can indicate fluctuations in pressure in one or both of the chambers 114A and 114B. One or more sensors located at the pump 120 can be in fluid communication with one or both of the chambers 114A and 114B, and the sensors can be operative to determine pressure within the chambers 114A and 114B. The control box 124 can be configured to determine at least one vital sign (e.g., heartrate, respiratory rate) based on the pressure within the chamber 114A or the chamber 114B.

In some implementations, the control box 124 can analyze a pressure signal detected by one or more pressure sensors to determine a heartrate, respiration rate, and/or other vital signs of the user lying or sitting on the chamber 114A and/or 114B. More specifically, when a user lies on the bed 112 and is positioned over the chamber 114A, each of the user's heart beats, breaths, and other movements (e.g., hand, arm, leg, foot, or other gross body movements) can create a force on the bed 112 that is transmitted to the chamber 114A. As a result of the force input applied to the chamber 114A from the user's movement, a wave can propagate through the chamber 114A and into the pump 120. A pressure sensor located at the pump 120 can detect the wave, and thus the pressure signal outputted by the sensor can indicate a heartrate, respiratory rate, or other information regarding the user.

With regard to sleep state, the air bed system 100 can determine the user's sleep state by using various biometric signals such as heartrate, respiration, and/or movement of the user. While the user is sleeping, the processor 136 can receive one or more of the user's biometric signals (e.g., heartrate, respiration, motion, etc.) and can determine the user's present sleep state based on the received biometric signals. In some implementations, signals indicating fluctuations in pressure in one or both of the chambers 114A and 114B can be amplified and/or filtered to allow for more precise detection of heartrate and respiratory rate.

Sometimes, the processor 136 can also receive additional biometric signals of the user from one or more other sensors or sensor arrays that are positioned on or otherwise integrated into the air bed system 100. For example, one or more sensors can be attached or removably attached to a top surface of the air bed system 100 and configured to detect signals such as heartrate, respiration rate, and/or motion of the user. The processor 136 can then combine biometric signals received from pressure sensors located at the pump 120, the pressure transducer 146, and/or the sensors positioned throughout the air bed system 100 to generate accurate and more precise heartrate, respiratory rate, and other information about the user and the user's sleep quality.

Sometimes, the control box 124 can perform a pattern recognition algorithm or other calculation based on the amplified and filtered pressure signal(s) to determine the user's heartrate and/or respiratory rate. For example, the algorithm or calculation can be based on assumptions that a heartrate portion of the signal has a frequency in a range of 0.5-4.0 Hz and that a respiration rate portion of the signal has a frequency in a range of less than 1 Hz. Sometimes, the control box 124 can use one or more machine learning models to determine the user's heartrate, respiratory rate, or other health information. The models can be trained using training data that includes training pressure signals and expected heartrates and/or respiratory rates. Sometimes, the control box 124 can determine the user's heartrate, respiratory rate, or other health information by using a lookup table that corresponds to sensed pressure signals.

The control box 124 can also be configured to determine other characteristics of the user based on the received pressure signal, such as blood pressure, tossing and turning movements, rolling movements, limb movements, weight, presence or lack of presence of the user, and/or the identity of the user.

For example, the pressure transducer 146 can be used to monitor the air pressure in the chambers 114A and 114B of the bed 112. If the user on the bed 112 is not moving, the air pressure changes in the air chamber 114A or 114B can be relatively minimal, and can be attributable to respiration and/or heartbeat. When the user on the bed 112 is moving, however, the air pressure in the mattress can fluctuate by a much larger amount. Thus, the pressure signals generated by the pressure transducer 146 and received by the processor 136 can be filtered and indicated as corresponding to motion, heartbeat, or respiration. The processor 136 can also attribute such fluctuations in air pressure to sleep quality of the user. Such attributions can be determined based on applying one or more machine learning models and/or algorithms to the pressure signals generated by the pressure transducer 146. For example, if the user shifts and turns a lot during a sleep cycle (for example, in comparison to historic trends of the user's sleep cycles), the processor 136 can determine that the user experienced poor sleep during that particular sleep cycle.

In some implementations, rather than performing the data analysis in the control box 124 with the processor 136, a digital signal processor (DSP) can be provided to analyze the data collected by the pressure transducer 146. Alternatively, the data collected by the pressure transducer 146 can be sent to a cloud-based computing system for remote analysis.

In some implementations, the example air bed system 100 further includes a temperature controller configured to increase, decrease, or maintain a temperature of the bed 112, for example for the comfort of the user. For example, a pad (e.g., mat, layer, etc.) can be placed on top of or be part of the bed 112, or can be placed on top of or be part of one or both of the chambers 114A and 114B. Air can be pushed through the pad and vented to cool off the user on the bed 112. Additionally or alternatively, the pad can include a heating element that can be used to keep the user warm. In some implementations, the temperature controller can receive temperature readings from the pad. The temperature controller can determine whether the temperature readings are less than or greater than some threshold range and/or value. Based on this determination, the temperature controller can actuate components to push air through the pad to cool off the user or active the heating element. In some implementations, separate pads are used for different sides of the bed 112 (e.g., corresponding to the locations of the chambers 114A and 114B) to provide for differing temperature control for the different sides of the bed 112. Each pad can therefore be selectively controlled by the temperature controller to provide cooling or heating that is preferred by each of the users on the different sides of the bed 112. For example, a first user on a left side of the bed 112 can prefer to have their side of the bed 112 cooled during the night while a second user on a right side of the bed 112 can prefer to have their side of the bed 112 warmed during the night.

In some implementations, the user of the air bed system 100 can use an input device, such as the remote control 122 or a mobile device as described above, to input a desired temperature for a surface of the bed 112 (or for a portion of the surface of the bed 112, for example at a foot region, a lumbar or waist region, a shoulder region, and/or a head region of the bed 112). The desired temperature can be encapsulated in a command data structure that includes the desired temperature and also identifies the temperature controller as the desired component to be controlled. The command data structure can then be transmitted via Bluetooth or another suitable communication protocol (e.g., WIFI, a local network, etc.) to the processor 136. In various examples, the command data structure is encrypted before being transmitted. The temperature controller can then configure its elements to increase or decrease the temperature of the pad depending on the temperature input provided at the remote control 122 by the user.

In some implementations, data can be transmitted from a component back to the processor 136 or to one or more display devices, such as the display 126 of the remote controller 122. For example, the current temperature as determined by a sensor element of temperature controller, the pressure of the bed, the current position of the foundation or other information can be transmitted to control box 124. The control box 124 can then transmit the received information to the remote control 122, where the information can be displayed to the user (e.g., on the display 126). As described above, the control box 124 can also transmit the received information to a mobile device (e.g., smartphone, cellphone, laptop, tablet, computer, wearable device, or home automation device) to be displayed in a mobile application or other graphical user interface (GUI) to the user.

In some implementations, the example air bed system 100 further includes an adjustable foundation and an articulation controller configured to adjust the position of a bed (e.g., the bed 112) by adjusting the adjustable foundation that supports the bed. For example, the articulation controller can adjust the bed 112 from a flat position to a position in which a head portion of a mattress of the bed is inclined upward (e.g., to facilitate a user sitting up in bed and/or watching television). The bed 112 can also include multiple separately articulable sections. As an illustrative example, the bed 112 can include one or more of a head portion, a lumbar/waist portion, a leg portion, and/or a foot portion, all of which can be separately articulable. As another example, portions of the bed 112 corresponding to the locations of the chambers 114A and 114B can be articulated independently from each other, to allow one user positioned on the bed 112 surface to rest in a first position (e.g., a flat position or other desired position) while a second user rests in a second position (e.g., a reclining position with the head raised at an angle from the waist or another desired position). Separate positions can also be set for two different beds (e.g., two twin beds placed next to each other). The foundation of the bed 112 can include more than one zone that can be independently adjusted.

Sometimes, the bed 112 can be adjusted to one or more user-defined positions based on user input and/or user preferences. For example, the bed 112 can automatically adjust, by the articulation controller, to one or more user-defined settings. As another example, the user can control the articulation controller to adjust the bed 112 to one or more user-defined positions. Sometimes, the bed 112 can be adjusted to one or more positions that may provide the user with improved or otherwise improve sleep and sleep quality. For example, a head portion on one side of the bed 112 can be automatically articulated, by the articulation controller, when one or more sensors of the air bed system 100 detect that a user sleeping on that side of the bed 112 is snoring. As a result, the user's snoring can be mitigated so that the snoring does not wake up another user sleeping in the bed 112.

In some implementations, the bed 112 can be adjusted using one or more devices in communication with the articulation controller or instead of the articulation controller. For example, the user can change positions of one or more portions of the bed 112 using the remote control 122 described above. The user can also adjust the bed 112 using a mobile application or other graphical user interface presented at a mobile computing device of the user.

The articulation controller can also be configured to provide different levels of massage to one or more portions of the bed 112 for one or more users on the bed 112. The user(s) can also adjust one or more massage settings for different portions of the bed 112 using the remote control 122 and/or a mobile device in communication with the air bed system 100, as described above.

Example of a Bed in a Bedroom Environment

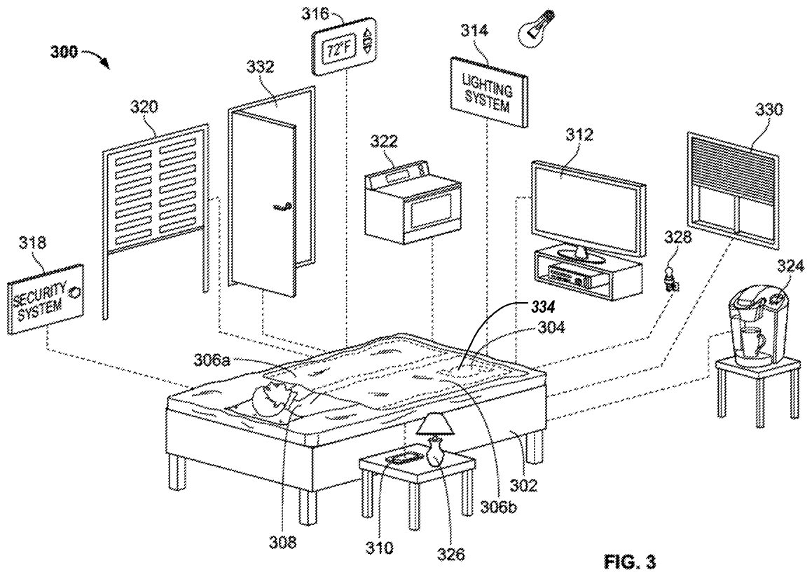

FIG. 3 shows an example environment 300 including a bed 302 in communication with devices located in and around a home. In the example shown, the bed 302 includes pump 304 for controlling air pressure within two air chambers 306a and 306b (as described above with respect to the air chambers 114A and 114B). The pump 304 additionally includes circuitry 334 for controlling inflation and deflation functionality performed by the pump 304. The circuitry 334 is further programmed to detect fluctuations in air pressure of the air chambers 306a-b and uses the detected fluctuations in air pressure to identify bed presence of a user 308, sleep state of the user 308, movement of the user 308, and biometric signals of the user 308, such as heartrate and respiration rate. The detected fluctuations in air pressure can also be used to detect when the user 308 is snoring and whether the user 308 has sleep apnea or other health conditions. Moreover, the detected fluctuations in air pressure can be used to determine an overall sleep quality of the user 308.

In the example shown, the pump 304 is located within a support structure of the bed 302 and the control circuitry 334 for controlling the pump 304 is integrated with the pump 304. In some implementations, the control circuitry 334 is physically separate from the pump 304 and is in wireless or wired communication with the pump 304. In some implementations, the pump 304 and/or control circuitry 334 are located outside of the bed 302. In some implementations, various control functions can be performed by systems located in different physical locations. For example, circuitry for controlling actions of the pump 304 can be located within a pump casing of the pump 304 while control circuitry 334 for performing other functions associated with the bed 302 can be located in another portion of the bed 302, or external to the bed 302. As another example, the control circuitry 334 located within the pump 304 can communicate with control circuitry 334 at a remote location through a LAN or WAN (e.g., the internet). As yet another example, the control circuitry 334 can be included in the control box 124 of FIGS. 1 and 2.

In some implementations, one or more devices other than, or in addition to, the pump 304 and control circuitry 334 can be utilized to identify user bed presence, sleep state, movement, biometric signals, and other information (e.g., sleep quality and/or health related) about the user 308. For example, the bed 302 can include a second pump in addition to the pump 304, with each of the two pumps connected to a respective one of the air chambers 306a-b. For example, the pump 304 can be in fluid communication with the air chamber 306b to control inflation and deflation of the air chamber 306b as well as detect user signals for a user located over the air chamber 306b, such as bed presence, sleep state, movement, and biometric signals. The second pump can then be in fluid communication with the air chamber 306a and used to control inflation and deflation of the air chamber 306a as well as detect user signals for a user located over the air chamber 306a.

As another example, the bed 302 can include one or more pressure sensitive pads or surface portions that are operable to detect movement, including user presence, user motion, respiration, and heartrate. A first pressure sensitive pad can be incorporated into a surface of the bed 302 over a left portion of the bed 302, where a first user would normally be located during sleep, and a second pressure sensitive pad can be incorporated into the surface of the bed 302 over a right portion of the bed 302, where a second user would normally be located during sleep. The movement detected by the one or more pressure sensitive pads or surface portions can be used by control circuitry 334 to identify user sleep state, bed presence, or biometric signals for each of the users. The pressure sensitive pads can also be removable rather than incorporated into the surface of the bed 302.

The bed 302 can also include one or more temperature sensors and/or array of sensors that are operable to detect temperatures in microclimates of the bed 302. Detected temperatures in different microclimates of the bed 302 can be used by the control circuitry 334 to determine one or more modifications to the user 308's sleep environment. For example, a temperature sensor located near a core region of the bed 302 where the user 308 rests can detect high temperature values. Such high temperature values can indicate that the user 308 is warm. To lower the user's body temperature in this microclimate, the control circuitry 334 can determine that a cooling element of the bed 302 can be activated. As another example, the control circuitry 334 can determine that a cooling unit in the home can be automatically activated to cool an ambient temperature in the environment 300.

The control circuitry 334 can also process a combination of signals sensed by different sensors that are integrated into, positioned on, or otherwise in communication with the bed 112. For example, pressure and temperature signals can be processed by the control circuitry 334 to more accurately determine one or more health conditions of the user 308 and/or sleep quality of the user 308. Acoustic signals detected by one or more microphones or other audio sensors can also be used in combination with pressure or motion sensors in order to determine when the user 308 snores, whether the user 308 has sleep apnea, and/or overall sleep quality of the user 308. Combinations of one or more other sensed signals are also possible for the control circuitry 334 to more accurately determine one or more health and/or sleep conditions of the user 308.

Accordingly, information detected by one or more sensors or other components of the bed 112 (e.g., motion information) can be processed by the control circuitry 334 and provided to one or more user devices, such as a user device 310 for presentation to the user 308 or to other users. The information can be presented in a mobile application or other graphical user interface at the user device 310. The user 308 can view different information that is processed and/or determined by the control circuitry 334 and based the signals that are detected by components of the bed 302. For example, the user 308 can view their overall sleep quality for a particular sleep cycle (e.g., the previous night), historic trends of their sleep quality, and health information. The user 308 can also adjust one or more settings of the bed 302 (e.g., increase or decrease pressure in one or more regions of the bed 302, incline or decline different regions of the bed 302, turn on or off massage features of the bed 302, etc.) using the mobile application that is presented at the user device 310.

In the example depicted in FIG. 3, the user device 310 is a mobile phone; however, the user device 310 can also be any one of a tablet, personal computer, laptop, a smartphone, a smart television (e.g., a television 312), a home automation device, or other user device capable of wired or wireless communication with the control circuitry 334, one or more other components of the bed 302, and/or one or more devices in the environment 300. The user device 310 can be in communication with the control circuitry 334 of the bed 302 through a network or through direct point-to-point communication. For example, the control circuitry 334 can be connected to a LAN (e.g., through a WIFI router) and communicate with the user device 310 through the LAN. As another example, the control circuitry 334 and the user device 310 can both connect to the Internet and communicate through the Internet. For example, the control circuitry 334 can connect to the Internet through a WIFI router and the user device 310 can connect to the Internet through communication with a cellular communication system. As another example, the control circuitry 334 can communicate directly with the user device 310 through a wireless communication protocol, such as Bluetooth. As yet another example, the control circuitry 334 can communicate with the user device 310 through a wireless communication protocol, such as ZigBee, Z-Wave, infrared, or another wireless communication protocol suitable for the application. As another example, the control circuitry 334 can communicate with the user device 310 through a wired connection such as, for example, a USB connector, serial/RS232, or another wired connection suitable for the application.

As mentioned above, the user device 310 can display a variety of information and statistics related to sleep, or user 308's interaction with the bed 302. For example, a user interface displayed by the user device 310 can present information including amount of sleep for the user 308 over a period of time (e.g., a single evening, a week, a month, etc.), amount of deep sleep, ratio of deep sleep to restless sleep, time lapse between the user 308 getting into bed and the user 308 falling asleep, total amount of time spent in the bed 302 for a given period of time, heartrate for the user 308 over a period of time, respiration rate for the user 308 over a period of time, or other information related to user interaction with the bed 302 by the user 308 or one or more other users of the bed 302. In some implementations, information for multiple users can be presented on the user device 310, for example information for a first user positioned over the air chamber 306a can be presented along with information for a second user positioned over the air chamber 306b. In some implementations, the information presented on the user device 310 can vary according to the age of the user 308. For example, the information presented on the user device 310 can evolve with the age of the user 308 such that different information is presented on the user device 310 as the user 308 ages as a child or an adult.

The user device 310 can also be used as an interface for the control circuitry 334 of the bed 302 to allow the user 308 to enter information and/or adjust one or more settings of the bed 302. The information entered by the user 308 can be used by the control circuitry 334 to provide better information to the user 308 or to various control signals for controlling functions of the bed 302 or other devices. For example, the user 308 can enter information such as weight, height, and age of the user 308. The control circuitry 334 can use this information to provide the user 308 with a comparison of the user 308's tracked sleep information to sleep information of other people having similar weights, heights, and/or ages as the user 308. The control circuitry 308 can also use this information to more accurately determine overall sleep quality and/or health of the user 308 based on information that is detected by one or more components (e.g., sensors) of the bed 302.

As another example, and as mentioned above, the user 308 can use the user device 310 as an interface for controlling air pressure of the air chambers 306a and 306b, for controlling various recline or incline positions of the bed 302, for controlling temperature of one or more surface temperature control devices of the bed 302, or for allowing the control circuitry 334 to generate control signals for other devices (as described in greater detail below).

In some implementations, the control circuitry 334 of the bed 302 can communicate with other devices or systems in addition to or instead of the user device 310. For example, the control circuitry 334 can communicate with the television 312, a lighting system 314, a thermostat 316, a security system 318, home automation devices, and/or other household devices, including but not limited to an oven 322, a coffee maker 324, a lamp 326, and/or a nightlight 328. Other examples of devices and/or systems that the control circuitry 334 can communicate with include a system for controlling window blinds 330, one or more devices for detecting or controlling the states of one or more doors 332 (such as detecting if a door is open, detecting if a door is locked, or automatically locking a door), and a system for controlling a garage door 320 (e.g., control circuitry 334 integrated with a garage door opener for identifying an open or closed state of the garage door 320 and for causing the garage door opener to open or close the garage door 320). Communications between the control circuitry 334 of the bed 302 and other devices can occur through a network (e.g., a LAN or the Internet) or as point-to-point communication (e.g., using Bluetooth, radio communication, or a wired connection). In some implementations, control circuitry 334 of different beds 302 can communicate with different sets of devices. For example, a kid's bed may not communicate with and/or control the same devices as an adult bed. In some embodiments, the bed 302 can evolve with the age of the user such that the control circuitry 334 of the bed 302 communicates with different devices as a function of age of the user of that bed 302.

The control circuitry 334 can receive information and inputs from other devices/systems and use the received information and inputs to control actions of the bed 302 and/or other devices. For example, the control circuitry 334 can receive information from the thermostat 316 indicating a current environmental temperature for a house or room in which the bed 302 is located. The control circuitry 334 can use the received information (along with other information, such as signals detected from one or more sensors of the bed 302) to determine if a temperature of all or a portion of the surface of the bed 302 should be raised or lowered. The control circuitry 334 can then cause a heating or cooling mechanism of the bed 302 to raise or lower the temperature of the surface of the bed 302. The control circuitry 334 can also cause a heating or cooling unit of the house or room in which the bed 302 is located to raise or lower the ambient temperature surrounding the bed 302. Thus, by adjusting the temperature of the bed 302 and/or the room in which the bed 302 is located, the user 308 can experience more improved sleep quality and comfort.

As an example, the user 308 can indicate a desired sleeping temperature of 74 degrees while a second user of the bed 302 indicates a desired sleeping temperature of 72 degrees. The thermostat 316 can transmit signals indicating room temperature at predetermined times to the control circuitry 334. The thermostat 316 can also send a continuous stream of detected temperature values of the room to the control circuitry 334. The transmitted signal(s) can indicate to the control circuitry 334 that the current temperature of the bedroom is 72 degrees. The control circuitry 334 can identify that the user 308 has indicated a desired sleeping temperature of 74 degrees, and can accordingly send control signals to a heating pad located on the user 308's side of the bed to raise the temperature of the portion of the surface of the bed 302 where the user 308 is located until the user 308's desired temperature is achieved. Moreover, the control circuitry 334 can sent control signals to the thermostat 316 and/or a heating unit in the house to raise the temperature in the room in which the bed 302 is located.

The control circuitry 334 can generate control signals to control other devices and propagate the control signals to the other devices. In some implementations, the control signals are generated based on information collected by the control circuitry 334, including information related to user interaction with the bed 302 by the user 308 and/or one or more other users. Information collected from one or more other devices other than the bed 302 can also be used when generating the control signals. For example, information relating to environmental occurrences (e.g., environmental temperature, environmental noise level, and environmental light level), time of day, time of year, day of the week, or other information can be used when generating control signals for various devices in communication with the control circuitry 334 of the bed 302.

For example, information on the time of day can be combined with information relating to movement and bed presence of the user 308 to generate control signals for the lighting system 314. The control circuitry 334 can, based on detected pressure signals of the user 308 on the bed 302, determine when the user 308 is presently in the bed 302 and when the user 308 falls asleep. Once the control circuitry 334 determines that the user has fallen asleep, the control circuitry 334 can transmit control signals to the lighting system 314 to turn off lights in the room in which the bed 302 is located, to lower the window blinds 330 in the room, and/or to activate the nightlight 328. Moreover, the control circuitry 334 can receive input from the user 308 (e.g., via the user device 310) that indicates a time at which the user 308 would like to wake up. When that time approaches, the control circuitry 334 can transmit control signals to one or more devices in the environment 300 to control devices that may cause the user 308 to wake up. For example, the control signals can be sent to a home automation device that controls multiple devices in the home. The home automation device can be instructed, by the control circuitry 334, to raise the window blinds 330, turn off the nightlight 328, turn on lighting beneath the bed 302, start the coffee machine 324, change a temperature in the house via the thermostat 316, or perform some other home automation. The home automation device can also be instructed to activate an alarm that can cause the user 308 to wake up. Sometimes, the user 308 can input information at the user device 310 that indicates what actions can be taken by the home automation device or other devices in the environment 300.

In some implementations, rather than or in addition to providing control signals for one or more other devices, the control circuitry 334 can provide collected information (e.g., information related to user movement, bed presence, sleep state, or biometric signals for the user 308) to one or more other devices to allow the one or more other devices to utilize the collected information when generating control signals. For example, the control circuitry 334 of the bed 302 can provide information relating to user interactions with the bed 302 by the user 308 to a central controller (not shown) that can use the provided information to generate control signals for various devices, including the bed 302.

The central controller can, for example, be a hub device that provides a variety of information about the user 308 and control information associated with the bed 302 and one or more other devices in the house. The central controller can include one or more sensors that detect signals that can be used by the control circuitry 334 and/or the central controller to determine information about the user 308 (e.g., biometric or other health data, sleep quality, etc.). The sensors can detect signals including but not limited to ambient light, temperature, humidity, volatile organic compound(s), pulse, motion, and audio. These signals can be combined with signals that are detected by sensors of the bed 302 to determine more accurate information about the user 308's health and sleep quality. The central controller can provide controls (e.g., user-defined, presets, automated, user initiated, etc.) for the bed 302, determining and viewing sleep quality and health information, a smart alarm clock, a speaker or other home automation device, a smart picture frame, a nightlight, and one or more mobile applications that the user 308 can install and use at the central controller. The central controller can include a display screen that can output information and also receive input from the user 308. The display can output information such as the user 308's health, sleep quality, weather information, security integration features, lighting integration features, heating and cooling integration features, and other controls to automate devices in the house. The central controller can therefore operate to provide the user 308 with functionality and control of multiple different types of devices in the house as well as the user 308's bed 302.

Still referring to FIG. 3, the control circuitry 334 of the bed 302 can generate control signals for controlling actions of other devices, and transmit the control signals to the other devices in response to information collected by the control circuitry 334, including bed presence of the user 308, sleep state of the user 308, and other factors. For example, the control circuitry 334 integrated with the pump 304 can detect a feature of a mattress of the bed 302, such as an increase in pressure in the air chamber 306b, and use this detected increase in air pressure to determine that the user 308 is present on the bed 302. In some implementations, the control circuitry 334 can identify a heartrate or respiratory rate for the user 308 to identify that the increase in pressure is due to a person sitting, laying, or otherwise resting on the bed 302, rather than an inanimate object (such as a suitcase) having been placed on the bed 302. In some implementations, the information indicating user bed presence can be combined with other information to identify a current or future likely state for the user 308. For example, a detected user bed presence at 11:00 am can indicate that the user is sitting on the bed (e.g., to tie her shoes, or to read a book) and does not intend to go to sleep, while a detected user bed presence at 10:00 pm can indicate that the user 308 is in bed for the evening and is intending to fall asleep soon. As another example, if the control circuitry 334 detects that the user 308 has left the bed 302 at 6:30 am (e.g., indicating that the user 308 has woken up for the day), and then later detects presence of the user 308 at 7:30 am on the bed 302, the control circuitry 334 can use this information that the newly detected presence is likely temporary (e.g., while the user 308 ties her shoes before heading to work) rather than an indication that the user 308 is intending to stay on the bed 302 for an extended period of time.

If the control circuitry 334 determines that the user 308 is likely to remain on the bed 302 for an extended period of time, the control circuitry 334 can determine one or more home automation controls that can aid the user 308 in falling asleep and experiencing improved sleep quality throughout the user 308's sleep cycle. For example, the control circuitry 334 can communicate with security system 318 to ensure that doors are locked. The control circuitry 334 can communicate with the oven 322 to ensure that the oven 322 is turned off. The control circuitry 334 can also communicate with the lighting system 314 to dim or otherwise turn off lights in the room in which the bed 302 is located and/or throughout the house, and the control circuitry 334 can communicate with the thermostat 316 to ensure that the house is at a desired temperature of the user 308. The control circuitry 334 can also determine one or more adjustments that can be made to the bed 302 to facilitate the user 308 falling asleep and staying asleep (e.g., changing a position of one or more regions of the bed 302, foot warming, massage features, pressure/firmness in one or more regions of the bed 302, etc.).

In some implementations, the control circuitry 334 is able to use collected information (including information related to user interaction with the bed 302 by the user 308, as well as environmental information, time information, and input received from the user 308) to identify use patterns for the user 308. For example, the control circuitry 334 can use information indicating bed presence and sleep states for the user 308 collected over a period of time to identify a sleep pattern for the user. The control circuitry 334 can identify that the user 308 generally goes to bed between 9:30 pm and 10:00 pm, generally falls asleep between 10:00 pm and 11:00 pm, and generally wakes up between 6:30 am and 6:45 am, based on information indicating user presence and biometrics for the user 308 collected over a week or a different time period. The control circuitry 334 can use identified patterns of the user 308 to better process and identify user interactions with the bed 302.

For example, given the above example user bed presence, sleep, and wake patterns for the user 308, if the user 308 is detected as being on the bed 302 at 3:00 pm, the control circuitry 334 can determine that the user 308's presence on the bed 302 is only temporary, and use this determination to generate different control signals than would be generated if the control circuitry 334 determined that the user 308 was in bed for the evening (e.g., at 3:00 pm, a head region of the bed 302 can be raised to facilitate reading or watching TV while in the bed 302, whereas in the evening, the bed 302 can be adjusted to a flat position to facilitate falling asleep). As another example, if the control circuitry 334 detects that the user 308 has gotten out of bed at 3:00 am, the control circuitry 334 can use identified patterns for the user 308 to determine that the user has only gotten up temporarily (e.g., to use the bathroom, or get a glass of water) and is not up for the day. For example, the control circuitry 334 can turn on underbed lighting to assist the user 308 in carefully moving around the bed 302 and the room. By contrast, if the control circuitry 334 identifies that the user 308 has gotten out of the bed 302 at 6:40 am, the control circuitry 334 can determine that the user 308 is up for the day and generate a different set of control signals than those that would be generated if it were determined that the user 308 were only getting out of bed temporarily (as would be the case when the user 308 gets out of the bed 302 at 3:00 am) (e.g., the control circuitry 334 can turn on light 326 near the bed 302 and/or raise the window blinds 330 when it is determined that the user 308 is up for the day). For other users, getting out of the bed 302 at 3:00 am can be a normal wake-up time, which the control circuitry 334 can learn and respond to accordingly. Moreover, if the bed 302 is occupied by two users, the control circuitry 334 can learn and respond to the patterns of each of the users.

As described above, the control circuitry 334 for the bed 302 can generate control signals for control functions of various other devices. The control signals can be generated, at least in part, based on detected interactions by the user 308 with the bed 302, as well as other information including time, date, temperature, etc. The control circuitry 334 can communicate with the television 312, receive information from the television 312, and generate control signals for controlling functions of the television 312. For example, the control circuitry 334 can receive an indication from the television 312 that the television 312 is currently turned on. If the television 312 is located in a different room than the bed 302, the control circuitry 334 can generate a control signal to turn the television 312 off upon making a determination that the user 308 has gone to bed for the evening or otherwise is remaining in the room with the bed 302. For example, if presence of the user 308 is detected on the bed 302 during a particular time range (e.g., between 8:00 pm and 7:00 am) and persists for longer than a threshold period of time (e.g., 10 minutes), the control circuitry 334 can determine that the user 308 is in bed for the evening. If the television 312 is on (as indicated by communications received by the control circuitry 334 of the bed 302 from the television 312), the control circuitry 334 can generate a control signal to turn the television 312 off. The control signals can be transmitted to the television (e.g., through a directed communication link between the television 312 and the control circuitry 334 or through a network, such as WIFI). As another example, rather than turning off the television 312 in response to detection of user bed presence, the control circuitry 334 can generate a control signal that causes the volume of the television 312 to be lowered by a pre-specified amount.

As another example, upon detecting that the user 308 has left the bed 302 during a specified time range (e.g., between 6:00 am and 8:00 am), the control circuitry 334 can generate control signals to cause the television 312 to turn on and tune to a pre-specified channel (e.g., the user 308 has indicated a preference for watching the morning news upon getting out of bed). The control circuitry 334 can generate the control signal and transmit the signal to the television 312 to cause the television 312 to turn on and tune to the desired station (which can be stored at the control circuitry 334, the television 312, or another location). As another example, upon detecting that the user 308 has gotten up for the day, the control circuitry 334 can generate and transmit control signals to cause the television 312 to turn on and begin playing a previously recorded program from a digital video recorder (DVR) in communication with the television 312.

As another example, if the television 312 is in the same room as the bed 302, the control circuitry 334 may not cause the television 312 to turn off in response to detection of user bed presence. Rather, the control circuitry 334 can generate and transmit control signals to cause the television 312 to turn off in response to determining that the user 308 is asleep. For example, the control circuitry 334 can monitor biometric signals of the user 308 (e.g., motion, heartrate, respiration rate) to determine that the user 308 has fallen asleep. Upon detecting that the user 308 is sleeping, the control circuitry 334 generates and transmits a control signal to turn the television 312 off. As another example, the control circuitry 334 can generate the control signal to turn off the television 312 after a threshold period of time has passed since the user 308 has fallen asleep (e.g., 10 minutes after the user has fallen asleep). As another example, the control circuitry 334 generates control signals to lower the volume of the television 312 after determining that the user 308 is asleep. As yet another example, the control circuitry 334 generates and transmits a control signal to cause the television to gradually lower in volume over a period of time and then turn off in response to determining that the user 308 is asleep. Any of the control signals described above in reference to the television 312 can also be determined by the central controller previously described.

In some implementations, the control circuitry 334 can similarly interact with other media devices, such as computers, tablets, mobile phones, smart phones, wearable devices, stereo systems, etc. For example, upon detecting that the user 308 is asleep, the control circuitry 334 can generate and transmit a control signal to the user device 310 to cause the user device 310 to turn off, or turn down the volume on a video or audio file being played by the user device 310.

The control circuitry 334 can additionally communicate with the lighting system 314, receive information from the lighting system 314, and generate control signals for controlling functions of the lighting system 314. For example, upon detecting user bed presence on the bed 302 during a certain time frame (e.g., between 8:00 pm and 7:00 am) that lasts for longer than a threshold period of time (e.g., 10 minutes), the control circuitry 334 of the bed 302 can determine that the user 308 is in bed for the evening. In response to this determination, the control circuitry 334 can generate control signals to cause lights in one or more rooms other than the room in which the bed 302 is located to switch off. The control signals can then be transmitted to the lighting system 314 and executed by the lighting system 314 to cause the lights in the indicated rooms to shut off. For example, the control circuitry 334 can generate and transmit control signals to turn off lights in all common rooms, but not in other bedrooms. As another example, the control signals generated by the control circuitry 334 can indicate that lights in all rooms other than the room in which the bed 302 is located are to be turned off, while one or more lights located outside of the house containing the bed 302 are to be turned on, in response to determining that the user 308 is in bed for the evening. Additionally, the control circuitry 334 can generate and transmit control signals to cause the nightlight 328 to turn on in response to determining user 308 bed presence or that the user 308 is asleep. As another example, the control circuitry 334 can generate first control signals for turning off a first set of lights (e.g., lights in common rooms) in response to detecting user bed presence, and second control signals for turning off a second set of lights (e.g., lights in the room in which the bed 302 is located) in response to detecting that the user 308 is asleep.

In some implementations, in response to determining that the user 308 is in bed for the evening, the control circuitry 334 of the bed 302 can generate control signals to cause the lighting system 314 to implement a sunset lighting scheme in the room in which the bed 302 is located. A sunset lighting scheme can include, for example, dimming the lights (either gradually over time, or all at once) in combination with changing the color of the light in the bedroom environment, such as adding an amber hue to the lighting in the bedroom. The sunset lighting scheme can help to put the user 308 to sleep when the control circuitry 334 has determined that the user 308 is in bed for the evening. Sometimes, the control signals can cause the lighting system 314 to dim the lights or change color of the lighting in the bedroom environment, but not both.

The control circuitry 334 can also be configured to implement a sunrise lighting scheme when the user 308 wakes up in the morning. The control circuitry 334 can determine that the user 308 is awake for the day, for example, by detecting that the user 308 has gotten off of the bed 302 (e.g., is no longer present on the bed 302) during a specified time frame (e.g., between 6:00 am and 8:00 am). As another example, the control circuitry 334 can monitor movement, heartrate, respiratory rate, or other biometric signals of the user 308 to determine that the user 308 is awake or is waking up, even though the user 308 has not gotten out of bed. If the control circuitry 334 detects that the user is awake or waking up during a specified timeframe, the control circuitry 334 can determine that the user 308 is awake for the day. The specified timeframe can be, for example, based on previously recorded user bed presence information collected over a period of time (e.g., two weeks) that indicates that the user 308 usually wakes up for the day between 6:30 am and 7:30 am. In response to the control circuitry 334 determining that the user 308 is awake, the control circuitry 334 can generate control signals to cause the lighting system 314 to implement the sunrise lighting scheme in the bedroom in which the bed 302 is located. The sunrise lighting scheme can include, for example, turning on lights (e.g., the lamp 326, or other lights in the bedroom). The sunrise lighting scheme can further include gradually increasing the level of light in the room where the bed 302 is located (or in one or more other rooms). The sunrise lighting scheme can also include only turning on lights of specified colors. For example, the sunrise lighting scheme can include lighting the bedroom with blue light to gently assist the user 308 in waking up and becoming active.

In some implementations, the control circuitry 334 can generate different control signals for controlling actions of one or more components, such as the lighting system 314, depending on a time of day that user interactions with the bed 302 are detected. For example, the control circuitry 334 can use historical user interaction information for interactions between the user 308 and the bed 302 to determine that the user 308 usually falls asleep between 10:00 pm and 11:00 pm and usually wakes up between 6:30 am and 7:30 am on weekdays. The control circuitry 334 can use this information to generate a first set of control signals for controlling the lighting system 314 if the user 308 is detected as getting out of bed at 3:00 am and to generate a second set of control signals for controlling the lighting system 314 if the user 308 is detected as getting out of bed after 6:30 am. For example, if the user 308 gets out of bed prior to 6:30 am, the control circuitry 334 can turn on lights that guide the user 308's route to a bathroom. As another example, if the user 308 gets out of bed prior to 6:30 am, the control circuitry 334 can turn on lights that guide the user 308's route to the kitchen (which can include, for example, turning on the nightlight 328, turning on under bed lighting, turning on the lamp 326, or turning on lights along a path that the user 308 takes to get to the kitchen).

As another example, if the user 308 gets out of bed after 6:30 am, the control circuitry 334 can generate control signals to cause the lighting system 314 to initiate a sunrise lighting scheme, or to turn on one or more lights in the bedroom and/or other rooms. In some implementations, if the user 308 is detected as getting out of bed prior to a specified morning rise time for the user 308, the control circuitry 334 can cause the lighting system 314 to turn on lights that are dimmer than lights that are turned on by the lighting system 314 if the user 308 is detected as getting out of bed after the specified morning rise time. Causing the lighting system 314 to only turn on dim lights when the user 308 gets out of bed during the night (e.g., prior to normal rise time for the user 308) can prevent other occupants of the house from being woken up by the lights while still allowing the user 308 to see in order to reach the bathroom, kitchen, or another destination in the house.

The historical user interaction information for interactions between the user 308 and the bed 302 can be used to identify user sleep and awake timeframes. For example, user bed presence times and sleep times can be determined for a set period of time (e.g., two weeks, a month, etc.). The control circuitry 334 can then identify a typical time range or timeframe in which the user 308 goes to bed, a typical timeframe for when the user 308 falls asleep, and a typical timeframe for when the user 308 wakes up (and in some cases, different timeframes for when the user 308 wakes up and when the user 308 actually gets out of bed). In some implementations, buffer time can be added to these timeframes. For example, if the user is identified as typically going to bed between 10:00 pm and 10:30 pm, a buffer of a half hour in each direction can be added to the timeframe such that any detection of the user getting in bed between 9:30 pm and 11:00 pm is interpreted as the user 308 going to bed for the evening. As another example, detection of bed presence of the user 308 starting from a half hour before the earliest typical time that the user 308 goes to bed extending until the typical wake up time (e.g., 6:30 am) for the user 308 can be interpreted as the user 308 going to bed for the evening. For example, if the user 308 typically goes to bed between 10:00 pm and 10:30 pm, if the user 308's bed presence is sensed at 12:30 am one night, that can be interpreted as the user 308 getting into bed for the evening even though this is outside of the user 308's typical timeframe for going to bed because it has occurred prior to the user 308's normal wake up time. In some implementations, different timeframes are identified for different times of the year (e.g., earlier bed time during winter vs. summer) or at different times of the week (e.g., user 308 wakes up earlier on weekdays than on weekends).

The control circuitry 334 can distinguish between the user 308 going to bed for an extended period (such as for the night) as opposed to being present on the bed 302 for a shorter period (such as for a nap) by sensing duration of presence of the user 308 (e.g., by detecting pressure signals and/or temperature signals of the user 308 on the bed 302 by one or more sensors that are integrated into the bed 302). In some examples, the control circuitry 334 can distinguish between the user 308 going to bed for an extended period (such as for the night) as opposed to going to bed for a shorter period (such as for a nap) by sensing duration of sleep of the user 308. For example, the control circuitry 334 can set a time threshold whereby if the user 308 is sensed on the bed 302 for longer than the threshold, the user 308 is considered to have gone to bed for the night. In some examples, the threshold can be about 2 hours, whereby if the user 308 is sensed on the bed 302 for greater than 2 hours, the control circuitry 334 registers that as an extended sleep event. In other examples, the threshold can be greater than or less than two hours. The threshold can also be determined based on historic trends indicating how long the user 302 usually sleeps or otherwise stays on the bed 302.

The control circuitry 334 can detect repeated extended sleep events to automatically determine a typical bed time range of the user 308, without requiring the user 308 to enter a bed time range. This can allow the control circuitry 334 to accurately estimate when the user 308 is likely to go to bed for an extended sleep event, regardless of whether the user 308 typically goes to bed using a traditional sleep schedule or a non-traditional sleep schedule. The control circuitry 334 can then use knowledge of the bed time range of the user 308 to control one or more components (including components of the bed 302 and/or non-bed peripherals) based on sensing bed presence during the bed time range or outside of the bed time range.

In some examples, the control circuitry 334 can automatically determine the bed time range of the user 308 without requiring user inputs. In some examples, the control circuitry 334 can determine the bed time range of the user 308 automatically and in combination with user inputs (e.g., using one or more signals that are sensed by sensors of the bed 302 and/or the central controller described above). In some examples, the control circuitry 334 can set the bed time range directly according to user inputs. In some examples, the control circuitry 334 can associate different bed times with different days of the week. In each of these examples, the control circuitry 334 can control one or more components (such as the lighting system 314, the thermostat 316, the security system 318, the oven 322, the coffee maker 324, the lamp 326, and the nightlight 328), as a function of sensed bed presence and the bed time range.

The control circuitry 334 can additionally communicate with the thermostat 316, receive information from the thermostat 316, and generate control signals for controlling functions of the thermostat 316. For example, the user 308 can indicate user preferences for different temperatures at different times, depending on the sleep state or bed presence of the user 308. For example, the user 308 may prefer an environmental temperature of 72 degrees when out of bed, 70 degrees when in bed but awake, and 68 degrees when sleeping. The control circuitry 334 of the bed 302 can detect bed presence of the user 308 in the evening and determine that the user 308 is in bed for the night. In response to this determination, the control circuitry 334 can generate control signals to cause the thermostat 316 to change the temperature to 70 degrees. The control circuitry 334 can then transmit the control signals to the thermostat 316. Upon detecting that the user 308 is in bed during the bed time range or asleep, the control circuitry 334 can generate and transmit control signals to cause the thermostat 316 to change the temperature to 68. The next morning, upon determining that the user 308 is awake for the day (e.g., the user 308 gets out of bed after 6:30 am), the control circuitry 334 can generate and transmit control circuitry 334 to cause the thermostat to change the temperature to 72 degrees.

The control circuitry 334 can also determine control signals to be transmitted to the thermostat 316 based on maintaining improved or preferred sleep quality of the user 308. In other words, the control circuitry 334 can determine adjustments to the thermostat 316 that are not merely based on user-inputted preferences. For example, the control circuitry 334 can determine, based on historic sleep patterns and quality of the user 308 and by applying one or more machine learning models, that the user 308 experiences their best sleep when the bedroom is at 74 degrees. The control circuitry 334 can receive temperature signals from one or more devices and/or sensors in the bedroom indicating a temperature of the bedroom. When the temperature is below 74 degrees, the control circuitry 334 can determine control signals that cause the thermostat 316 to activate a heating unit in the house to raise the temperature to 74 degrees in the bedroom. When the temperature is above 74 degrees, the control circuitry 334 can determine control signals that cause the thermostat 316 to activate a cooling unit in the house to lower the temperature back to 74 degrees. Sometimes, the control circuitry 334 can also determine control signals that cause the thermostat 316 to maintain the bedroom within a temperature range that is intended to keep the user 308 in particular sleep states and/or transition to next preferred sleep states.

In some implementations, the control circuitry 334 can generate control signals to cause one or more heating or cooling elements on the surface of the bed 302 to change temperature at various times, either in response to user interaction with the bed 302, at various pre-programmed times, based on user preference, and/or in response to detecting microclimate temperatures of the user 308 on the bed 302. For example, the control circuitry 334 can activate a heating element to raise the temperature of one side of the surface of the bed 302 to 73 degrees when it is detected that the user 308 has fallen asleep. As another example, upon determining that the user 308 is up for the day, the control circuitry 334 can turn off a heating or cooling element. As yet another example, the user 308 can pre-program various times at which the temperature at the surface of the bed should be raised or lowered. For example, the user 308 can program the bed 302 to raise the surface temperature to 76 degrees at 10:00 pm, and lower the surface temperature to 68 degrees at 11:30 pm. As another example, one or more temperature sensors on the surface of the bed 302 can detect microclimates of the user 308 on the bed 302. When a detected microclimate of the user 308 drops below a predetermined threshold temperature, the control circuitry 334 can activate a heating element to raise the user 308's body temperature, thereby improving the user 308's comfortability, maintaining the user 308 in their sleep cycle, transitioning the user 308 to a next preferred sleep state, and/or otherwise maintaining or improving the user 308's sleep quality.

In some implementations, in response to detecting user bed presence of the user 308 and/or that the user 308 is asleep, the control circuitry 334 can cause the thermostat 316 to change the temperature in different rooms to different values. For example, in response to determining that the user 308 is in bed for the evening, the control circuitry 334 can generate and transmit control signals to cause the thermostat 316 to set the temperature in one or more bedrooms of the house to 72 degrees and set the temperature in other rooms to 67 degrees. Other control signals are also possible, and can be based on user preference and user input.

The control circuitry 334 can also receive temperature information from the thermostat 316 and use this temperature information to control functions of the bed 302 or other devices. For example, as discussed above, the control circuitry 334 can adjust temperatures of heating elements included in or otherwise attached to the bed 302 (e.g., a foot warming pad) in response to temperature information received from the thermostat 316.

In some implementations, the control circuitry 334 can generate and transmit control signals for controlling other temperature control systems. For example, in response to determining that the user 308 is awake for the day, the control circuitry 334 can generate and transmit control signals for causing floor heating elements to activate in the bedroom and/or in other rooms in the house. For example, the control circuitry 334 can cause a floor heating system in a master bedroom to turn on in response to determining that the user 308 is awake for the day. One or more of the control signals described herein that are determined by the control circuitry 334 can also be determined by the central controller described above.

The control circuitry 334 can additionally communicate with the security system 318, receive information from the security system 318, and generate control signals for controlling functions of the security system 318. For example, in response to detecting that the user 308 in is bed for the evening, the control circuitry 334 can generate control signals to cause the security system 318 to engage or disengage security functions. The control circuitry 334 can then transmit the control signals to the security system 318 to cause the security system 318 to engage (e.g., turning on security cameras along a perimeter of the house, automatically locking doors in the house, etc.). As another example, the control circuitry 334 can generate and transmit control signals to cause the security system 318 to disable in response to determining that the user 308 is awake for the day (e.g., user 308 is no longer present on the bed 302 after 6:00 am). In some implementations, the control circuitry 334 can generate and transmit a first set of control signals to cause the security system 318 to engage a first set of security features in response to detecting user bed presence of the user 308, and can generate and transmit a second set of control signals to cause the security system 318 to engage a second set of security features in response to detecting that the user 308 has fallen asleep.

In some implementations, the control circuitry 334 can receive alerts from the security system 318 and indicate the alert to the user 308. For example, the control circuitry 334 can detect that the user 308 is in bed for the evening and in response, generate and transmit control signals to cause the security system 318 to engage or disengage. The security system can then detect a security breach (e.g., someone has opened the door 332 without entering the security code, or someone has opened a window when the security system 318 is engaged). The security system 318 can communicate the security breach to the control circuitry 334 of the bed 302. In response to receiving the communication from the security system 318, the control circuitry 334 can generate control signals to alert the user 308 to the security breach. For example, the control circuitry 334 can cause the bed 302 to vibrate. As another example, the control circuitry 334 can cause portions of the bed 302 to articulate (e.g., cause the head section to raise or lower) in order to wake the user 308 and alert the user to the security breach. As another example, the control circuitry 334 can generate and transmit control signals to cause the lamp 326 to flash on and off at regular intervals to alert the user 308 to the security breach. As another example, the control circuitry 334 can alert the user 308 of one bed 302 regarding a security breach in a bedroom of another bed, such as an open window in a kid's bedroom. As another example, the control circuitry 334 can send an alert to a garage door controller (e.g., to close and lock the door). As another example, the control circuitry 334 can send an alert for the security to be disengaged. The control circuitry 334 can also set off a smart alarm or other alarm device/clock near the bed 302. The control circuitry 334 can transmit a push notification, text message, or other indication of the security breach to the user device 310. Also, the control circuitry 334 can transmit a notification of the security breach to the central controller described above The central controller can then determine one or more responses to the security breach.

The control circuitry 334 can additionally generate and transmit control signals for controlling the garage door 320 and receive information indicating a state of the garage door 320 (e.g., open or closed). For example, in response to determining that the user 308 is in bed for the evening, the control circuitry 334 can generate and transmit a request to a garage door opener or another device capable of sensing if the garage door 320 is open. The control circuitry 334 can request information on the current state of the garage door 320. If the control circuitry 334 receives a response (e.g., from the garage door opener) indicating that the garage door 320 is open, the control circuitry 334 can either notify the user 308 that the garage door is open (e.g., by displaying a notification or other message at the user device 310, by outputting a notification at the central controller, etc.), and/or generate a control signal to cause the garage door opener to close the garage door 320. For example, the control circuitry 334 can send a message to the user device 310 indicating that the garage door is open. As another example, the control circuitry 334 can cause the bed 302 to vibrate. As yet another example, the control circuitry 334 can generate and transmit a control signal to cause the lighting system 314 to cause one or more lights in the bedroom to flash to alert the user 308 to check the user device 310 for an alert (in this example, an alert regarding the garage door 320 being open). Alternatively, or additionally, the control circuitry 334 can generate and transmit control signals to cause the garage door opener to close the garage door 320 in response to identifying that the user 308 is in bed for the evening and that the garage door 320 is open. Control signals can also vary depend on the age of the user 308.

The control circuitry 334 can similarly send and receive communications for controlling or receiving state information associated with the door 332 or the oven 322. For example, upon detecting that the user 308 is in bed for the evening, the control circuitry 334 can generate and transmit a request to a device or system for detecting a state of the door 332. Information returned in response to the request can indicate various states of the door 332 such as open, closed but unlocked, or closed and locked. If the door 332 is open or closed but unlocked, the control circuitry 334 can alert the user 308 to the state of the door, such as in a manner described above with reference to the garage door 320. Alternatively, or in addition to alerting the user 308, the control circuitry 334 can generate and transmit control signals to cause the door 332 to lock, or to close and lock. If the door 332 is closed and locked, the control circuitry 334 can determine that no further action is needed.

Similarly, upon detecting that the user 308 is in bed for the evening, the control circuitry 334 can generate and transmit a request to the oven 322 to request a state of the oven 322 (e.g., on or off). If the oven 322 is on, the control circuitry 334 can alert the user 308 and/or generate and transmit control signals to cause the oven 322 to turn off. If the oven is already off, the control circuitry 334 can determine that no further action is necessary. In some implementations, different alerts can be generated for different events. For example, the control circuitry 334 can cause the lamp 326 (or one or more other lights, via the lighting system 314) to flash in a first pattern if the security system 318 has detected a breach, flash in a second pattern if garage door 320 is on, flash in a third pattern if the door 332 is open, flash in a fourth pattern if the oven 322 is on, and flash in a fifth pattern if another bed has detected that a user 308 of that bed has gotten up (e.g., that a child of the user 308 has gotten out of bed in the middle of the night as sensed by a sensor in the child's bed). Other examples of alerts that can be processed by the control circuitry 334 of the bed 302 and communicated to the user (e.g., at the user device 310 and/or the central controller described herein) include a smoke detector detecting smoke (and communicating this detection of smoke to the control circuitry 334), a carbon monoxide tester detecting carbon monoxide, a heater malfunctioning, or an alert from any other device capable of communicating with the control circuitry 334 and detecting an occurrence that should be brought to the user 308's attention.

The control circuitry 334 can also communicate with a system or device for controlling a state of the window blinds 330. For example, in response to determining that the user 308 is in bed for the evening, the control circuitry 334 can generate and transmit control signals to cause the window blinds 330 to close. As another example, in response to determining that the user 308 is up for the day (e.g., user has gotten out of bed after 6:30 am) or that the user 308 set an alarm to wake up at a particular time, the control circuitry 334 can generate and transmit control signals to cause the window blinds 330 to open. By contrast, if the user 308 gets out of bed prior to a normal rise time for the user 308, the control circuitry 334 can determine that the user 308 is not awake for the day and may not generate control signals that cause the window blinds 330 to open. As yet another example, the control circuitry 334 can generate and transmit control signals that cause a first set of blinds to close in response to detecting user bed presence of the user 308 and a second set of blinds to close in response to detecting that the user 308 is asleep.

The control circuitry 334 can generate and transmit control signals for controlling functions of other household devices in response to detecting user interactions with the bed 302. For example, in response to determining that the user 308 is awake for the day, the control circuitry 334 can generate and transmit control signals to the coffee maker 324 to cause the coffee maker 324 to begin brewing coffee. As another example, the control circuitry 334 can generate and transmit control signals to the oven 322 to cause the oven 322 to begin preheating (for users that like fresh baked bread in the morning or otherwise bake or prepare food in the morning). As another example, the control circuitry 334 can use information indicating that the user 308 is awake for the day along with information indicating that the time of year is currently winter and/or that the outside temperature is below a threshold value to generate and transmit control signals to cause a car engine block heater to turn on.

As another example, the control circuitry 334 can generate and transmit control signals to cause one or more devices to enter a sleep mode in response to detecting user bed presence of the user 308, or in response to detecting that the user 308 is asleep. For example, the control circuitry 334 can generate control signals to cause a mobile phone of the user 308 to switch into sleep mode or night mode such that notifications from the mobile phone are muted to not disturb the user 308's sleep. The control circuitry 334 can then transmit the control signals to the mobile phone. Later, upon determining that the user 308 is up for the day, the control circuitry 334 can generate and transmit control signals to cause the mobile phone to switch out of sleep mode.

In some implementations, the control circuitry 334 can communicate with one or more noise control devices. For example, upon determining that the user 308 is in bed for the evening, or that the user 308 is asleep (e.g., based on pressure signals received from the bed 302, audio/decibel signals received from audio sensors positioned on or around the bed 302, etc.), the control circuitry 334 can generate and transmit control signals to cause one or more noise cancelation devices to activate. The noise cancelation devices can, for example, be included as part of the bed 302 or located in the bedroom with the bed 302. As another example, upon determining that the user 308 is in bed for the evening or that the user 308 is asleep, the control circuitry 334 can generate and transmit control signals to turn the volume on, off, up, or down, for one or more sound generating devices, such as a stereo system radio, television, computer, tablet, mobile phone, etc.

Additionally, functions of the bed 302 can be controlled by the control circuitry 334 in response to user interactions with the bed 302. As mentioned throughout, functions of the bed 302 described herein can also be controlled by the user device 310 and/or the central controller (e.g., a hub device or other home automation device that controls multiple different devices in the home). As mentioned above, the bed 302 can include an adjustable foundation and an articulation controller configured to adjust the position of one or more portions of the bed 302 by adjusting the adjustable foundation that supports the bed 302. For example, the articulation controller can adjust the bed 302 from a flat position to a position in which a head portion of a mattress of the bed 302 is inclined upward (e.g., to facilitate a user sitting up in bed, reading, and/or watching television). In some implementations, the bed 302 includes multiple separately articulable sections. For example, portions of the bed corresponding to the locations of the air chambers 306a and 306b can be articulated independently from each other, to allow one person positioned on the bed 302 surface to rest in a first position (e.g., a flat position) while a second person rests in a second position (e.g., a reclining position with the head raised at an angle from the waist). In some implementations, separate positions can be set for two different beds (e.g., two twin beds placed next to each other). The foundation of the bed 302 can include more than one zone that can be independently adjusted. The articulation controller can also be configured to provide different levels of massage to one or more users on the bed 302 or to cause the bed to vibrate to communicate alerts to the user 308 as described above.

The control circuitry 334 can adjust positions (e.g., incline and decline positions for the user 308 and/or an additional user of the bed 302) in response to user interactions with the bed 302. For example, the control circuitry 334 can cause the articulation controller to adjust the bed 302 to a first recline position for the user 308 in response to sensing user bed presence for the user 308. The control circuitry 334 can cause the articulation controller to adjust the bed 302 to a second recline position (e.g., a less reclined, or flat position) in response to determining that the user 308 is asleep. As another example, the control circuitry 334 can receive a communication from the television 312 indicating that the user 308 has turned off the television 312, and in response, the control circuitry 334 can cause the articulation controller to adjust the position of the bed 302 to a preferred user sleeping position (e.g., due to the user turning off the television 312 while the user 308 is in bed indicating that the user 308 wishes to go to sleep).

In some implementations, the control circuitry 334 can control the articulation controller so as to wake up one user of the bed 302 without waking another user of the bed 302. For example, the user 308 and a second user of the bed 302 can each set distinct wakeup times (e.g., 6:30 am and 7:15 am respectively). When the wakeup time for the user 308 is reached, the control circuitry 334 can cause the articulation controller to vibrate or change the position of only a side of the bed on which the user 308 is located to wake the user 308 without disturbing the second user. When the wakeup time for the second user is reached, the control circuitry 334 can cause the articulation controller to vibrate or change the position of only the side of the bed on which the second user is located. Alternatively, when the second wakeup time occurs, the control circuitry 334 can utilize other methods (such as audio alarms, or turning on the lights) to wake the second user since the user 308 is already awake and therefore will not be disturbed when the control circuitry 334 attempts to wake the second user.

Still referring to FIG. 3, the control circuitry 334 for the bed 302 can utilize information for interactions with the bed 302 by multiple users to generate control signals for controlling functions of various other devices. For example, the control circuitry 334 can wait to generate control signals for, for example, engaging the security system 318, or instructing the lighting system 314 to turn off lights in various rooms, until both the user 308 and a second user are detected as being present on the bed 302. As another example, the control circuitry 334 can generate a first set of control signals to cause the lighting system 314 to turn off a first set of lights upon detecting bed presence of the user 308 and generate a second set of control signals for turning off a second set of lights in response to detecting bed presence of a second user. As another example, the control circuitry 334 can wait until it has been determined that both the user 308 and a second user are awake for the day before generating control signals to open the window blinds 330. As yet another example, in response to determining that the user 308 has left the bed 302 and is awake for the day, but that a second user is still sleeping, the control circuitry 334 can generate and transmit a first set of control signals to cause the coffee maker 324 to begin brewing coffee, to cause the security system 318 to deactivate, to turn on the lamp 326, to turn off the nightlight 328, to cause the thermostat 316 to raise the temperature in one or more rooms to 72 degrees, and/or to open the window blinds 330 in rooms other than the bedroom in which the bed 302 is located. Later, in response to detecting that the second user is no longer present on the bed (or that the second user is awake or is waking up) the control circuitry 334 can generate and transmit a second set of control signals to, for example, cause the lighting system 314 to turn on one or more lights in the bedroom, to cause window blinds in the bedroom to open, and to turn on the television 312 to a pre-specified channel. One or more other home automation control signals can be determined and generated by the control circuitry 334, the user device 310, and/or the central controller described herein.

Examples of Data Processing Systems Associated with a Bed

Described here are examples of systems and components that can be used for data processing tasks that are, for example, associated with a bed. In some cases, multiple examples of a particular component or group of components are presented. Some of these examples are redundant and/or mutually exclusive alternatives. Connections between components are shown as examples to illustrate possible network configurations for allowing communication between components. Different formats of connections can be used as technically needed or desired. The connections generally indicate a logical connection that can be created with any technologically feasible format. For example, a network on a motherboard can be created with a printed circuit board, wireless data connections, and/or other types of network connections. Some logical connections are not shown for clarity. For example, connections with power supplies and/or computer readable memory may not be shown for clarities sake, as many or all elements of a particular component may need to be connected to the power supplies and/or computer readable memory.

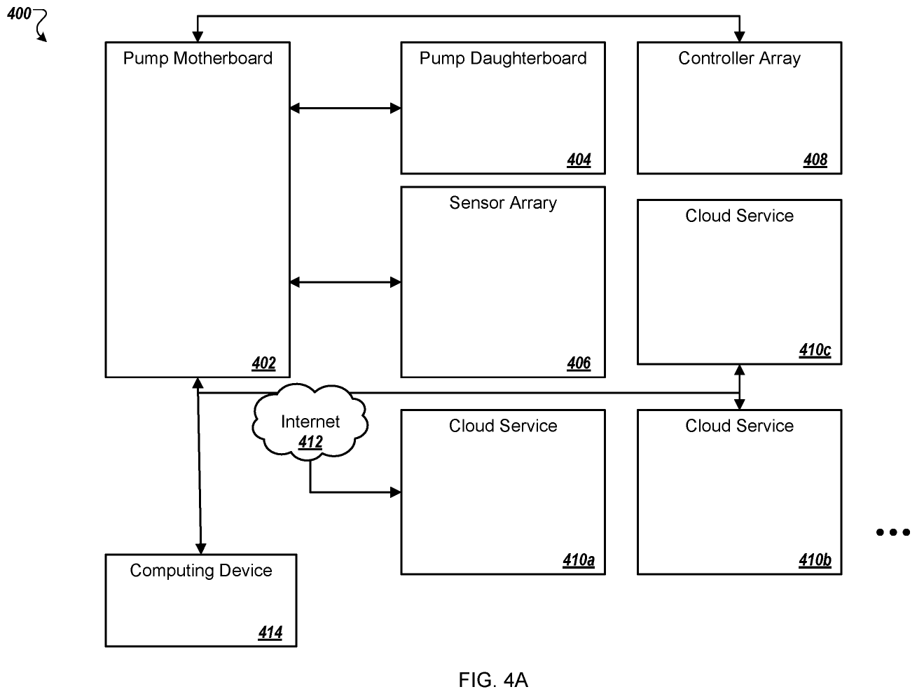

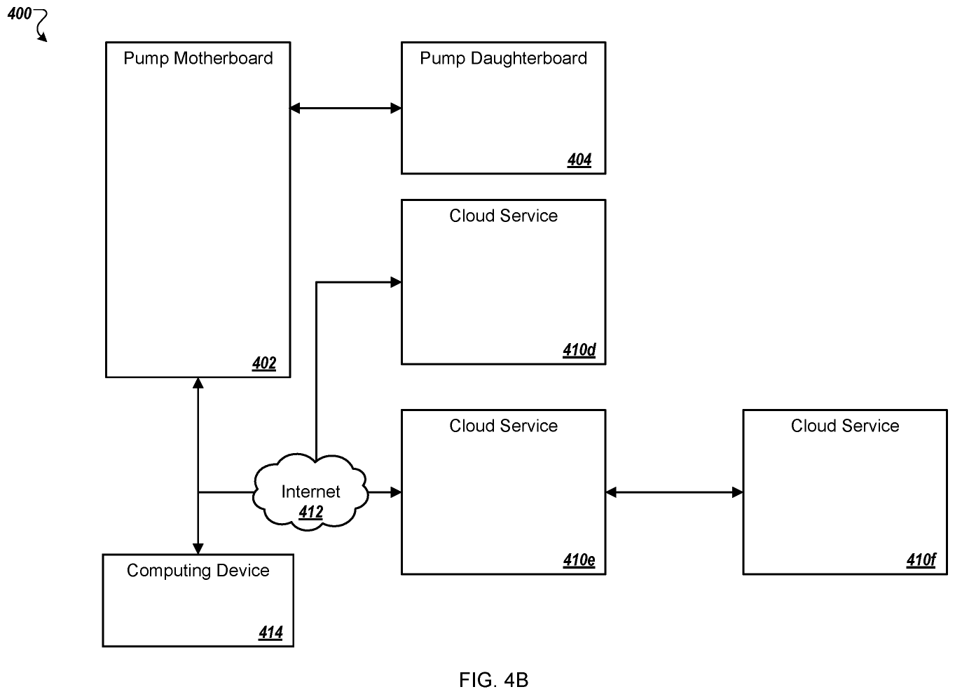

FIG. 4A is a block diagram of an example of a data processing system 400 that can be associated with a bed system, including those described above with respect to FIGS. 1-3. This system 400 includes a pump motherboard 402 and a pump daughterboard 404. The system 400 includes a sensor array 406 that can include one or more sensors configured to sense physical phenomenon of the environment and/or bed, and to report such sensing back to the pump motherboard 402 for, for example, analysis. The sensor array 406 can include one or more different types of sensors, including but not limited to pressure sensors, temperature sensors, light sensors, movement (e.g. motion) sensors, and audio sensors. The system 400 also includes a controller array 408 that can include one or more controllers configured to control logic-controlled devices of the bed and/or environment (such as home automation devices, security systems light systems, and other devices that are described in reference to FIG. 3). The pump motherboard 400 can be in communication with one or more computing devices 414 and one or more cloud services 410 over local networks, the Internet 412, or otherwise as is technically appropriate. Each of these components will be described in more detail, some with multiple example configurations, below.

In this example, a pump motherboard 402 and a pump daughterboard 404 are communicably coupled. They can be conceptually described as a center or hub of the system 400, with the other components conceptually described as spokes of the system 400. In some configurations, this can mean that each of the spoke components communicates primarily or exclusively with the pump motherboard 402. For example, a sensor of the sensor array 406 may not be configured to, or may not be able to, communicate directly with a corresponding controller. Instead, each spoke component can communicate with the motherboard 402. The sensor of the sensor array 406 can report a sensor reading to the motherboard 402, and the motherboard 402 can determine that, in response, a controller of the controller array 408 should adjust some parameters of a logic controlled device or otherwise modify a state of one or more peripheral devices. In one case, if the temperature of the bed is determined to be too hot based on received temperature signals from the sensor array 406, the pump motherboard 402 can determine that a temperature controller should cool the bed.

One advantage of a hub-and-spoke network configuration, sometimes also referred to as a star-shaped network, is a reduction in network traffic compared to, for example, a mesh network with dynamic routing. If a particular sensor generates a large, continuous stream of traffic, that traffic may only be transmitted over one spoke of the network to the motherboard 402. The motherboard 402 can, for example, marshal that data and condense it to a smaller data format for retransmission for storage in a cloud service 410. Additionally or alternatively, the motherboard 402 can generate a single, small, command message to be sent down a different spoke of the network in response to the large stream. For example, if the large stream of data is a pressure reading that is transmitted from the sensor array 406 a few times a second, the motherboard 402 can respond with a single command message to the controller array to increase the pressure in an air chamber of the bed. In this case, the single command message can be orders of magnitude smaller than the stream of pressure readings.

As another advantage, a hub-and-spoke network configuration can allow for an extensible network that can accommodate components being added, removed, failing, etc. This can allow, for example, more, fewer, or different sensors in the sensor array 406, controllers in the controller array 408, computing devices 414, and/or cloud services 410. For example, if a particular sensor fails or is deprecated by a newer version of the sensor, the system 400 can be configured such that only the motherboard 402 needs to be updated about the replacement sensor. This can allow, for example, product differentiation where the same motherboard 402 can support an entry level product with fewer sensors and controllers, a higher value product with more sensors and controllers, and customer personalization where a customer can add their own selected components to the system 400.

Additionally, a line of air bed products can use the system 400 with different components. In an application in which every air bed in the product line includes both a central logic unit and a pump, the motherboard 402 (and optionally the daughterboard 404) can be designed to fit within a single, universal housing. Then, for each upgrade of the product in the product line, additional sensors, controllers, cloud services, etc., can be added. Design, manufacturing, and testing time can be reduced by designing all products in a product line from this base, compared to a product line in which each product has a bespoke logic control system.

Each of the components discussed above can be realized in a wide variety of technologies and configurations. Below, some examples of each component will be further discussed. In some alternatives, two or more of the components of the system 400 can be realized in a single alternative component; some components can be realized in multiple, separate components; and/or some functionality can be provided by different components.

FIG. 4B is a block diagram showing some communication paths of the data processing system 400. As previously described, the motherboard 402 and the pump daughterboard 404 may act as a hub for peripheral devices and cloud services of the system 400. In cases in which the pump daughterboard 404 communicates with cloud services or other components, communications from the pump daughterboard 404 may be routed through the pump motherboard 402. This may allow, for example, the bed to have only a single connection with the internet 412. The computing device 414 may also have a connection to the internet 412, possibly through the same gateway used by the bed and/or possibly through a different gateway (e.g., a cell service provider).

Previously, a number of cloud services 410 were described. As shown in FIG. 4B, some cloud services, such as cloud services 410d and 410e, may be configured such that the pump motherboard 402 can communicate with the cloud service directly—that is the motherboard 402 may communicate with a cloud service 410 without having to use another cloud service 410 as an intermediary. Additionally or alternatively, some cloud services 410, for example cloud service 410f, may only be reachable by the pump motherboard 402 through an intermediary cloud service, for example cloud service 410e. While not shown here, some cloud services 410 may be reachable either directly or indirectly by the pump motherboard 402.

Additionally, some or all of the cloud services 410 may be configured to communicate with other cloud services. This communication may include the transfer of data and/or remote function calls according to any technologically appropriate format. For example, one cloud service 410 may request a copy for another cloud service's 410 data, for example, for purposes of backup, coordination, migration, or for performance of calculations or data mining. In another example, many cloud services 410 may contain data that is indexed according to specific users tracked by the user account cloud 410c and/or the bed data cloud 410a. These cloud services 410 may communicate with the user account cloud 410c and/or the bed data cloud 410a when accessing data specific to a particular user or bed.

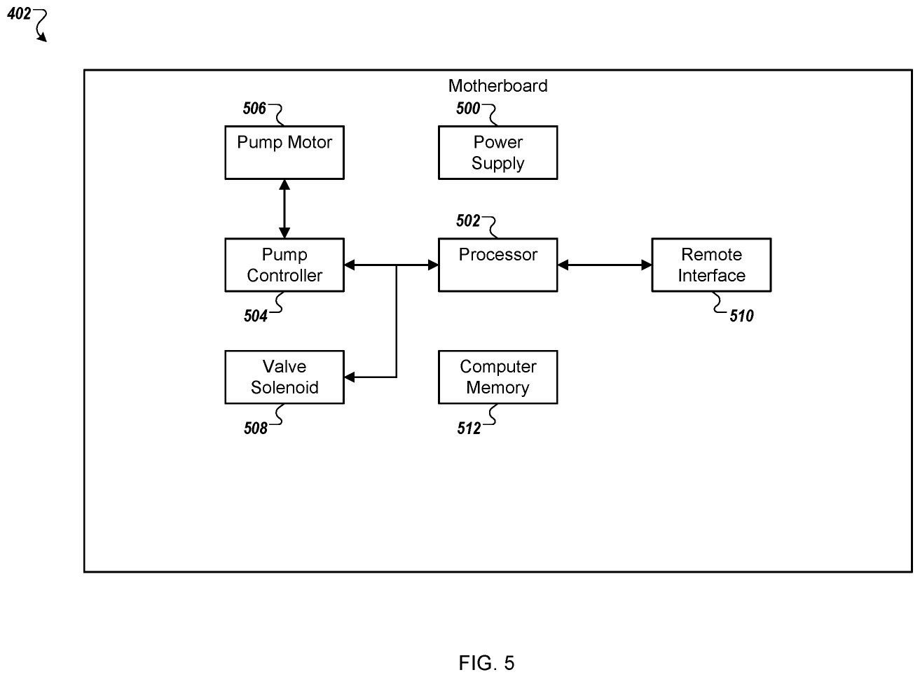

FIG. 5 is a block diagram of an example of a motherboard 402 that can be used in a data processing system that can be associated with a bed system, including those described above with respect to FIGS. 1-3. In this example, compared to other examples described below, this motherboard 402 consists of relatively fewer parts and can be limited to provide a relatively limited feature set.

The motherboard 402 includes a power supply 500, a processor 502, and computer memory 512. In general, the power supply 500 includes hardware used to receive electrical power from an outside source and supply it to components of the motherboard 402. The power supply can include, for example, a battery pack and/or wall outlet adapter, an AC to DC converter, a DC to AC converter, a power conditioner, a capacitor bank, and/or one or more interfaces for providing power in the current type, voltage, etc., needed by other components of the motherboard 402.

The processor 502 is generally a device for receiving input, performing logical determinations, and providing output. The processor 502 can be a central processing unit, a microprocessor, general purpose logic circuitry, application-specific integrated circuitry, a combination of these, and/or other hardware for performing the functionality needed.

The memory 512 is generally one or more devices for storing data. The memory 512 can include long term stable data storage (e.g., on a hard disk), short term unstable (e.g., on Random Access Memory) or any other technologically appropriate configuration.

The motherboard 402 includes a pump controller 504 and a pump motor 506. The pump controller 504 can receive commands from the processor 502 and, in response, control the functioning of the pump motor 506. For example, the pump controller 504 can receive, from the processor 502, a command to increase pressure of an air chamber by 0.3 pounds per square inch (PSI). The pump controller 504, in response, engages a valve so that the pump motor 506 is configured to pump air into the selected air chamber, and can engage the pump motor 506 for a length of time that corresponds to 0.3 PSI or until a sensor indicates that pressure has been increased by 0.3 PSI. In an alternative configuration, the message can specify that the chamber should be inflated to a target PSI, and the pump controller 504 can engage the pump motor 506 until the target PSI is reached.

A valve solenoid 508 can control which air chamber a pump is connected to. In some cases, the solenoid 508 can be controlled by the processor 502 directly. In some cases, the solenoid 508 can be controlled by the pump controller 504.

A remote interface 510 of the motherboard 402 can allow the motherboard 402 to communicate with other components of a data processing system. For example, the motherboard 402 can be able to communicate with one or more daughterboards, with peripheral sensors, and/or with peripheral controllers through the remote interface 510. The remote interface 510 can provide any technologically appropriate communication interface, including but not limited to multiple communication interfaces such as WIFI, Bluetooth, and copper wired networks.

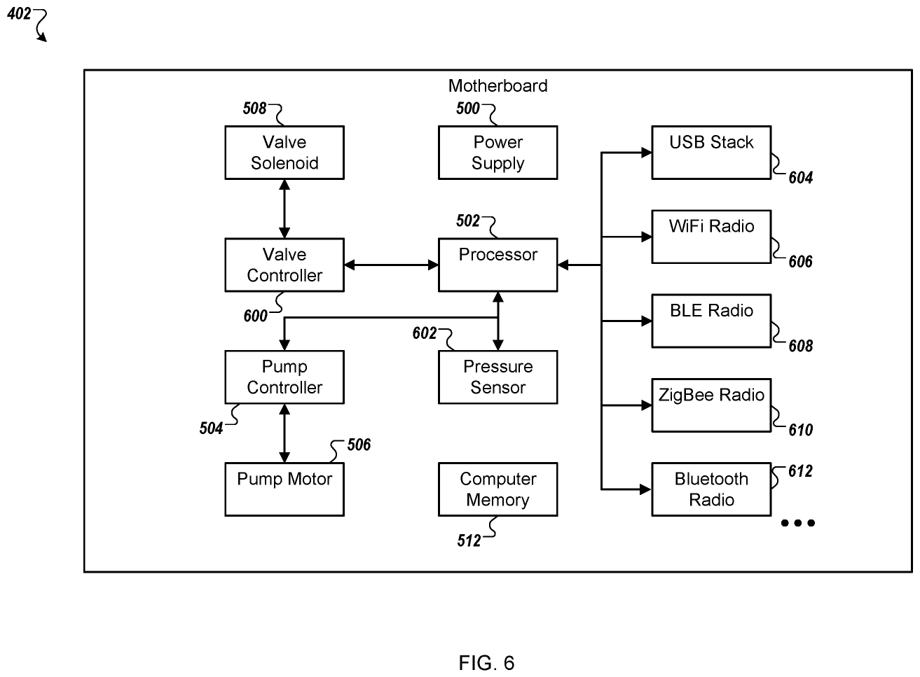

FIG. 6 is a block diagram of an example of the motherboard 402 that can be used in a data processing system associated with a bed system, including those described above with respect to FIGS. 1-3. Compared to the motherboard 402 described with reference to FIG. 5, the motherboard 402 in FIG. 6 can contain more components and provide more functionality in some applications.

In addition to the power supply 500, processor 502, pump controller 504, pump motor 506, and valve solenoid 508, this motherboard 402 is shown with a valve controller 600, a pressure sensor 602, a universal serial bus (USB) stack 604, a WiFi radio 606, a Bluetooth Low Energy (BLE) radio 608, a ZigBee radio 610, a Bluetooth radio 612, and a computer memory 512.

Similar to the way that the pump controller 504 converts commands from the processor 502 into control signals for the pump motor 506, the valve controller 600 can convert commands from the processor 502 into control signals for the valve solenoid 508. In one example, the processor 502 can issue a command to the valve controller 600 to connect the pump to a particular air chamber out of a group of air chambers in an air bed. The valve controller 600 can control the position of the valve solenoid 508 so that the pump is connected to the indicated air chamber.

The pressure sensor 602 can read pressure readings from one or more air chambers of the air bed. The pressure sensor 602 can also preform digital sensor conditioning. As described herein, multiple pressure sensors 602 can be included as part of the motherboard 402 or otherwise in communication with the motherboard 402.

The motherboard 402 can include a suite of network interfaces 604, 606, 608, 610, 612, etc., including but not limited to those shown in FIG. 6. These network interfaces can allow the motherboard to communicate over a wired or wireless network with any number of devices, including but not limited to peripheral sensors, peripheral controllers, computing devices, and devices and services connected to the Internet 412.

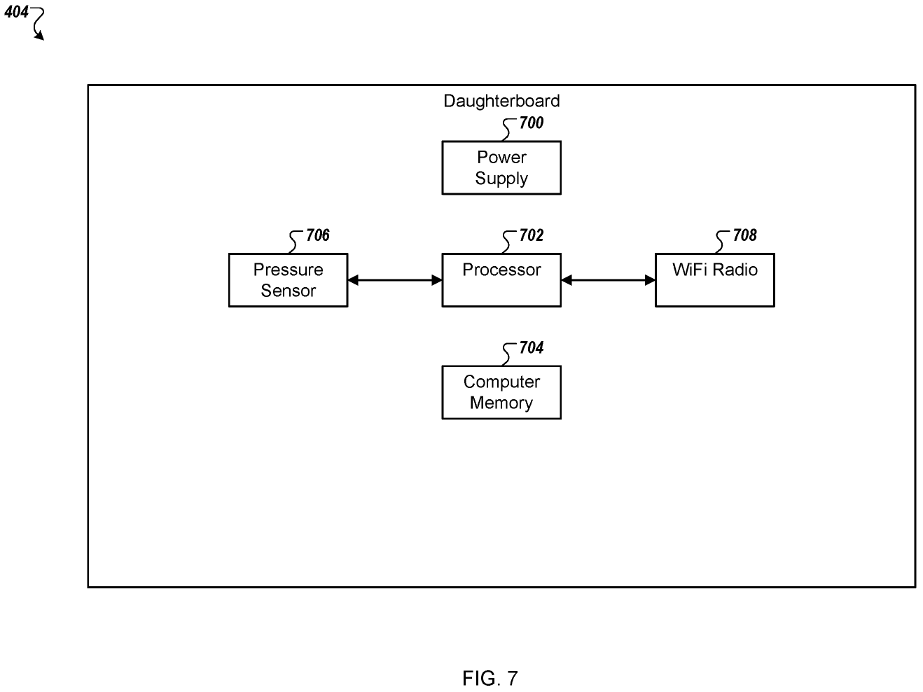

FIG. 7 is a block diagram of an example of a daughterboard 404 that can be used in a data processing system associated with a bed system, including those described above with respect to FIGS. 1-3. In some configurations, one or more daughterboards 404 can be connected to the motherboard 402. Some daughterboards 404 can be designed to offload particular and/or compartmentalized tasks from the motherboard 402. This can be advantageous, for example, if the particular tasks are computationally intensive, proprietary, or subject to future revisions. For example, the daughterboard 404 can be used to calculate a particular sleep data metric. This metric can be computationally intensive, and calculating the sleep metric on the daughterboard 404 can free up the resources of the motherboard 402 while the metric is being calculated. Additionally and/or alternatively, the sleep metric can be subject to future revisions. To update the system 400 with the new sleep metric, it is possible that only the daughterboard 404 that calculates that metric need be replaced. In this case, the same motherboard 402 and other components can be used, saving the need to perform unit testing of additional components instead of just the daughterboard 404.

The daughterboard 404 is shown with a power supply 700, a processor 702, computer readable memory 704, a pressure sensor 706, and a WiFi radio 708. The processor 702 can use the pressure sensor 706 to gather information about the pressure of an air chamber or chambers of an air bed. From this data, the processor 702 can perform an algorithm to calculate a sleep metric (e.g., sleep quality, whether a user is presently in the bed, whether the user has fallen asleep, a heartrate of the user, a respiration rate of the user, movement of the user, etc.). In some examples, the sleep metric can be calculated from only the pressure of air chambers. In other examples, the sleep metric can be calculated using signals from a variety of sensors (e.g., a movement sensor, a pressure sensor, a temperature sensor, and/or an audio sensor). In an example in which different data is needed, the processor 702 can receive that data from an appropriate sensor or sensors. These sensors can be internal to the daughterboard 404, accessible via the WiFi radio 708, or otherwise in communication with the processor 702. Once the sleep metric is calculated, the processor 702 can report that sleep metric to, for example, the motherboard 402. The motherboard 402 can then generate instructions for outputting the sleep metric to the user or otherwise using the sleep metric to determine one or more other information about the user or controls to control the bed system and/or peripheral devices.

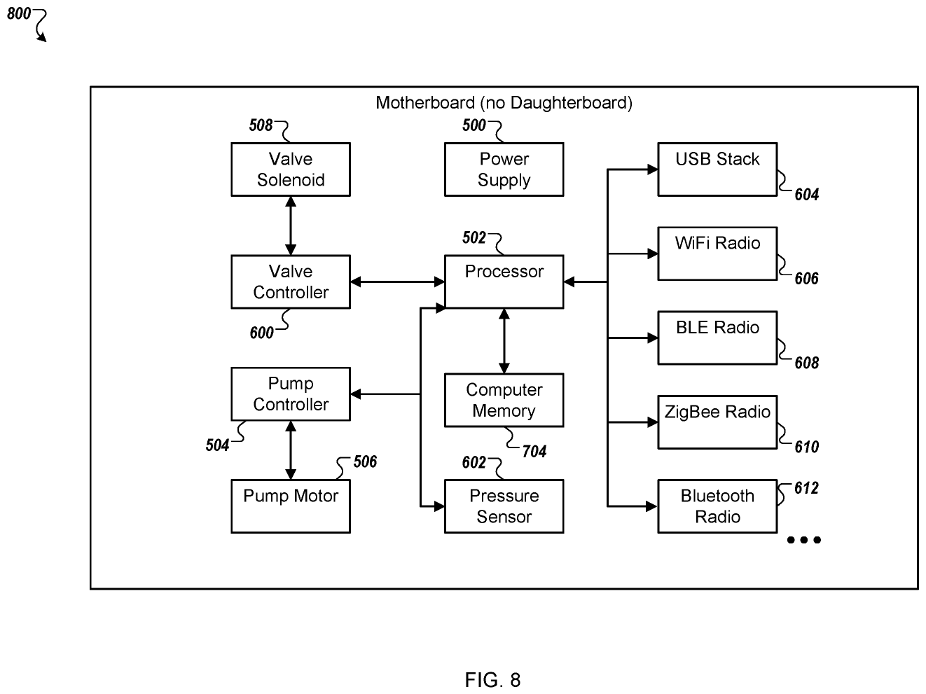

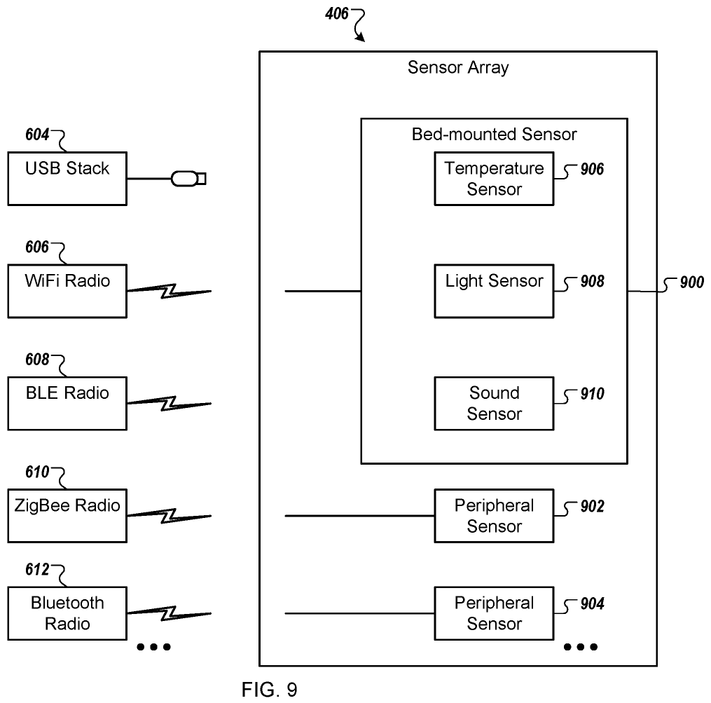

FIG. 8 is a block diagram of an example of a motherboard 800 with no daughterboard that can be used in a data processing system associated with a bed system, including those described above with respect to FIGS. 1-3. In this example, the motherboard 800 can perform most, all, or more of the features described with reference to the motherboard 402 in FIG. 6 and the daughterboard 404 in FIG. 7.

FIG. 9 is a block diagram of an example of the sensory array 406 that can be used in a data processing system associated with a bed system, including those described above with respect to FIGS. 1-3. In general, the sensor array 406 is a conceptual grouping of some or all the peripheral sensors that communicate with the motherboard 402 but are not native to the motherboard 402.

The peripheral sensors 902, 904, 906, 908, 910, etc. of the sensor array 406 can communicate with the motherboard 402 through one or more of the network interfaces of the motherboard, including but not limited to the USB stack 604, WiFi radio 606, Bluetooth Low Energy (BLE) radio 608, ZigBee radio 610, and Bluetooth radio 612, as is appropriate for the configuration of the particular sensor. For example, a sensor that outputs a reading over a USB cable can communicate through the USB stack 604.

Some of the peripheral sensors of the sensor array 406 can be bed mounted sensors 900, such as a temperature sensor 906, a light sensor 908, and a sound sensor 910. The bed mounted sensors 900 can be, for example, embedded into the structure of a bed and sold with the bed, or later affixed to the structure of the bed (e.g., part of a pressure sensing pad that is removably installed on a top surface of the bed, part of a temperature sensing or heating pad that is removably installed on the top surface of the bed, integrated into the top surface of the bed, attached along connecting tubes between a pump and air chambers, within air chambers, attached to a headboard of the bed, attached to one or more regions of an adjustable foundation, etc.). Other sensors 902 and 904 can be in communication with the motherboard 402, but optionally not mounted to the bed. The other sensors 902 and 904 can include a pressure sensor 902 and/or peripheral sensor 904. For example, the sensors 902 and 904 can be integrated or otherwise part of a user mobile device (e.g., mobile phone, wearable device, etc.). The sensors 902 and 904 can also be part of a central controller for controlling the bed and peripheral devices in the home. Sometimes, the sensors 902 and 904 can also be part of one or more home automation devices or other peripheral devices in the home.

In some cases, some or all of the bed mounted sensors 900 and/or sensors 902 and 904 can share networking hardware, including a conduit that contains wires from each sensor, a multi-wire cable or plug that, when affixed to the motherboard 402, connect all of the associated sensors with the motherboard 402. In some embodiments, one, some, or all of sensors 902, 904, 906, 908, and 910 can sense one or more features of a mattress, such as pressure, temperature, light, sound, and/or one or more other features of the mattress. In some embodiments, one, some, or all of sensors 902, 904, 906, 908, and 910 can sense one or more features external to the mattress. In some embodiments, pressure sensor 902 can sense pressure of the mattress while some or all of sensors 902, 904, 906, 908, and 910 can sense one or more features of the mattress and/or external to the mattress.

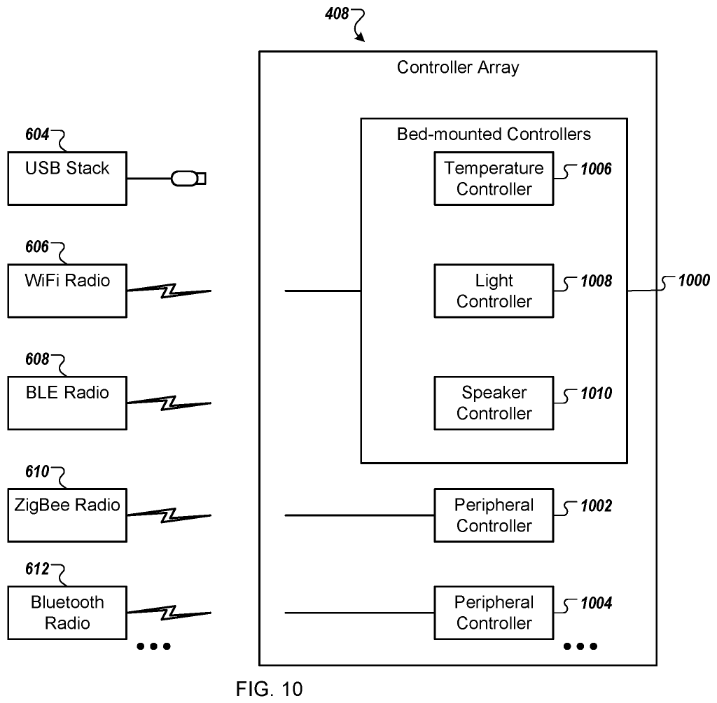

FIG. 10 is a block diagram of an example of the controller array 408 that can be used in a data processing system associated with a bed system, including those described above with respect to FIGS. 1-3. In general, the controller array 408 is a conceptual grouping of some or all peripheral controllers that communicate with the motherboard 402 but are not native to the motherboard 402.

The peripheral controllers of the controller array 408 can communicate with the motherboard 402 through one or more of the network interfaces of the motherboard, including but not limited to the USB stack 604, WiFi radio 606, Bluetooth Low Energy (BLE) radio 608, ZigBee radio 610, and Bluetooth radio 612, as is appropriate for the configuration of the particular sensor. For example, a controller that receives a command over a USB cable can communicate through the USB stack 604.

Some of the controllers of the controller array 408 can be bed mounted controllers 1000, such as a temperature controller 1006, a light controller 1008, and a speaker controller 1010. The bed mounting controllers 1000 can be, for example, embedded into the structure of a bed and sold with the bed, or later affixed to the structure of the bed, as described in reference to the peripheral sensors in FIG. 9. Other peripheral controllers 1002 and 1004 can be in communication with the motherboard 402, but optionally not mounted to the bed. In some cases, some or all of the bed mounted controllers 1000 and/or the peripheral controllers 1002 and 1004 can share networking hardware, including a conduit that contains wires for each controller, a multi-wire cable or plug that, when affixed to the motherboard 402, connects all of the associated controllers with the motherboard 402.

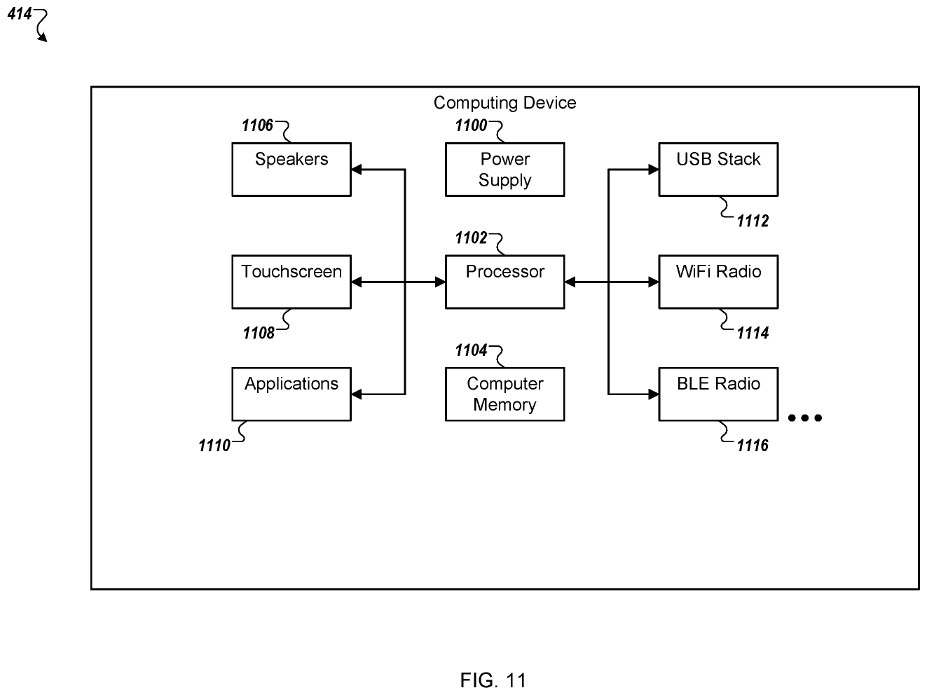

FIG. 11 is a block diagram of an example of the computing device 412 that can be used in a data processing system associated with a bed system, including those described above with respect to FIGS. 1-3. The computing device 412 can include, for example, computing devices used by a user of a bed. Example computing devices 412 include, but are not limited to, mobile computing devices (e.g., mobile phones, tablet computers, laptops, smart phones, wearable devices), desktop computers, home automation devices, and/or central controllers or other hub devices.

The computing device 412 includes a power supply 1100, a processor 1102, and computer readable memory 1104. User input and output can be transmitted by, for example, speakers 1106, a touchscreen 1108, or other not shown components, such as a pointing device or keyboard. The computing device 412 can run one or more applications 1110. These applications can include, for example, applications to allow the user to interact with the system 400. These applications can allow a user to view information about the bed (e.g., sensor readings, sleep metrics), information about themselves (e.g., health conditions that are detected based on signals that are sensed at the bed), and/or configure the behavior of the system 400 (e.g., set a desired firmness to the bed, set desired behavior for peripheral devices). In some cases, the computing device 412 can be used in addition to, or to replace, the remote control 122 described previously.

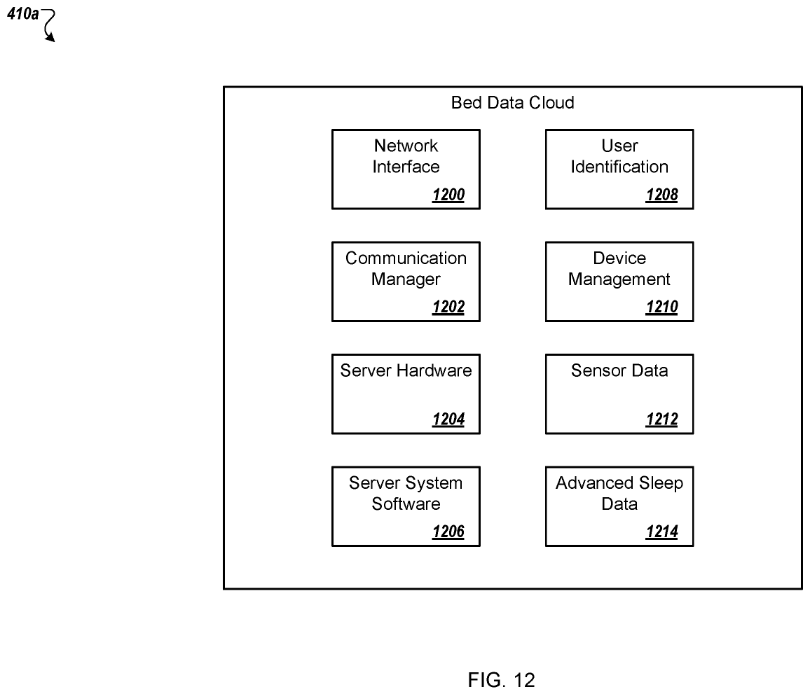

FIG. 12 is a block diagram of an example bed data cloud service 410a that can be used in a data processing system associated with a bed system, including those described above with respect to FIGS. 1-3. In this example, the bed data cloud service 410a is configured to collect sensor data and sleep data from a particular bed, and to match the sensor and sleep data with one or more users that use the bed when the sensor and sleep data was generated.

The bed data cloud service 410a is shown with a network interface 1200, a communication manager 1202, server hardware 1204, and server system software 1206. In addition, the bed data cloud service 410a is shown with a user identification module 1208, a device management 1210 module, a sensor data module 1210, and an advanced sleep data module 1214.

The network interface 1200 generally includes hardware and low level software used to allow one or more hardware devices to communicate over networks. For example the network interface 1200 can include network cards, routers, modems, and other hardware needed to allow the components of the bed data cloud service 410a to communicate with each other and other destinations over, for example, the Internet 412.

The communication manager 1202 generally comprises hardware and software that operate above the network interface 1200. This includes software to initiate, maintain, and tear down network communications used by the bed data cloud service 410a. This includes, for example, TCP/IP, SSL or TLS, Torrent, and other communication sessions over local or wide area networks. The communication manager 1202 can also provide load balancing and other services to other elements of the bed data cloud service 410a.

The server hardware 1204 generally includes physical processing devices used to instantiate and maintain the bed data cloud service 410a. This hardware includes, but is not limited to, processors (e.g., central processing units, ASICs, graphical processers) and computer readable memory (e.g., random access memory, stable hard disks, tape backup). One or more servers can be configured into clusters, multi-computer, or datacenters that can be geographically separate or connected.

The server system software 1206 generally includes software that runs on the server hardware 1204 to provide operating environments to applications and services. The server system software 1206 can include operating systems running on real servers, virtual machines instantiated on real servers to create many virtual servers, server level operations such as data migration, redundancy, and backup.

The user identification 1208 can include, or reference, data related to users of beds with associated data processing systems. For example, the users can include customers, owners, or other users registered with the bed data cloud service 410a or another service. Each user can have, for example, a unique identifier, user credentials, contact information, billing information, demographic information, or any other technologically appropriate information.

The device manager 1210 can include, or reference, data related to beds or other products associated with data processing systems. For example, the beds can include products sold or registered with a system associated with the bed data cloud service 410a. Each bed can have, for example, a unique identifier, model and/or serial number, sales information, geographic information, delivery information, a listing of associated sensors and control peripherals, etc. Additionally, an index or indexes stored by the bed data cloud service 410a can identify users that are associated with beds. For example, this index can record sales of a bed to a user, users that sleep in a bed, etc.

The sensor data 1212 can record raw or condensed sensor data recorded by beds with associated data processing systems. For example, a bed's data processing system can have a temperature sensor, pressure sensor, motion sensor, audio sensor, and/or light sensor. Readings from one or more of these sensors, either in raw form or in a format generated from the raw data (e.g. sleep metrics) of the sensors, can be communicated by the bed's data processing system to the bed data cloud service 410a for storage in the sensor data 1212. Additionally, an index or indexes stored by the bed data cloud service 410a can identify users and/or beds that are associated with the sensor data 1212.

The bed data cloud service 410a can use any of its available data, such as the sensor data 1212, to generate advanced sleep data 1214. In general, the advanced sleep data 1214 includes sleep metrics and other data generated from sensor readings, such as health information associated with the user of a particular bed. Some of these calculations can be performed in the bed data cloud service 410a instead of locally on the bed's data processing system, for example, because the calculations can be computationally complex or require a large amount of memory space or processor power that may not be available on the bed's data processing system. This can help allow a bed system to operate with a relatively simple controller and still be part of a system that performs relatively complex tasks and computations.

For example, the bed data cloud service 410a can retrieve one or more machine learning models from a remote data store and use those models to determine the advanced sleep data 1214. The bed data cloud service 410a can retrieve different types of models based on a type of the advanced sleep data 1214 that is being generated. As an illustrative example, the bed data cloud service 410a can retrieve one or more models to determine overall sleep quality of the user based on currently detected sensor data 1212 and/or historic sensor data (e.g., which can be stored in and accessed from a data store). The bed data cloud service 410a can retrieve one or more other models to determine whether the user is currently snoring based on the detected sensor data 1212. The bed data cloud service 410a can also retrieve one or more other models that can be used to determine whether the user is experiencing some health condition based on the detected sensor data 1212.

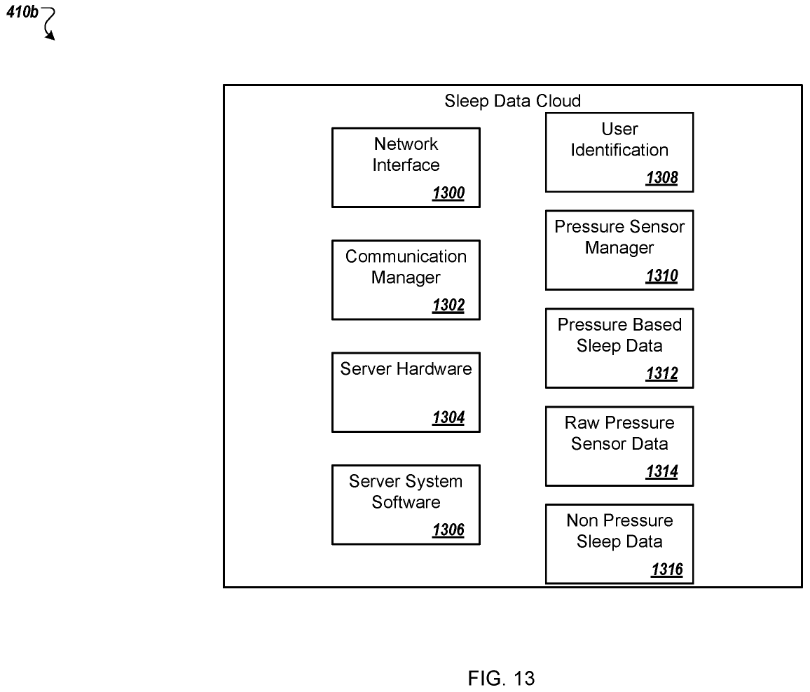

FIG. 13 is a block diagram of an example sleep data cloud service 410b that can be used in a data processing system associated with a bed system, including those described above with respect to FIGS. 1-3. In this example, the sleep data cloud service 410b is configured to record data related to users' sleep experience.

The sleep data cloud service 410b is shown with a network interface 1300, a communication manager 1302, server hardware 1304, and server system software 1306. In addition, the sleep data cloud service 410b is shown with a user identification module 1308, a pressure sensor manager 1310, a pressure based sleep data module 1312, a raw pressure sensor data module 1314, and a non-pressure sleep data module 1316. Sometimes, the sleep data cloud service 410b can include a sensor manager for each of the sensors that are integrated or otherwise in communication with the bed. In some implementations, the sleep data cloud service 410b can include a sensor manager that relates to multiple sensors in beds. For example, a single sensor manager can relate to pressure, temperature, light, movement, and audio sensors in a bed.

Referring to the sleep data cloud service 410b in FIG. 13, the pressure sensor manager 1310 can include, or reference, data related to the configuration and operation of pressure sensors in beds. For example, this data can include an identifier of the types of sensors in a particular bed, their settings and calibration data, etc.

The pressure based sleep data 1312 can use raw pressure sensor data 1314 to calculate sleep metrics specifically tied to pressure sensor data. For example, user presence, movements, weight change, heartrate, and breathing rate can all be determined from raw pressure sensor data 1314. Additionally, an index or indexes stored by the sleep data cloud service 410b can identify users that are associated with pressure sensors, raw pressure sensor data, and/or pressure based sleep data.

The non-pressure sleep data 1316 can use other sources of data to calculate sleep metrics. For example, user-entered preferences, light sensor readings, and sound sensor readings can all be used to track sleep data. Additionally, an index or indexes stored by the sleep data cloud service 410b can identify users that are associated with other sensors and/or non-pressure sleep data 1316.

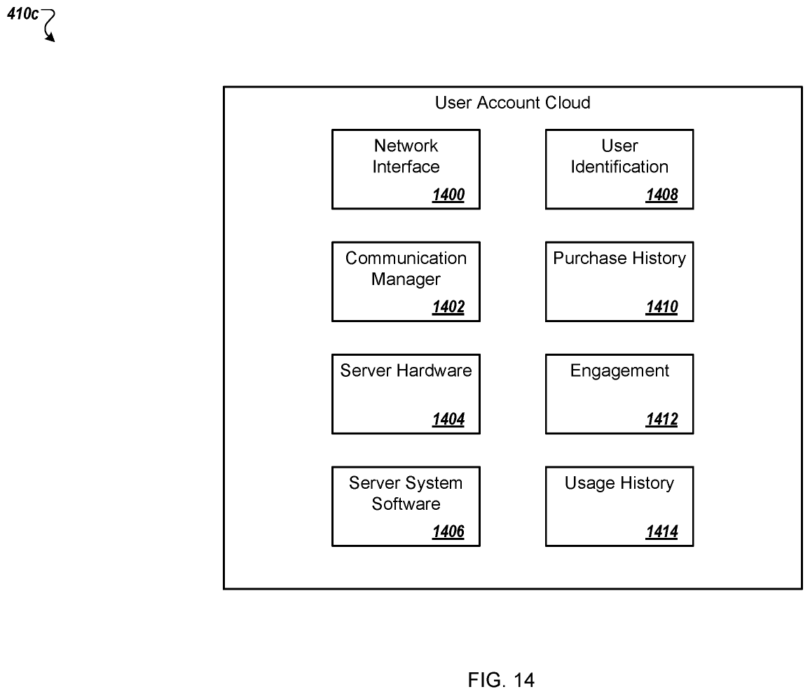

FIG. 14 is a block diagram of an example user account cloud service 410c that can be used in a data processing system associated with a bed system, including those described above with respect to FIGS. 1-3. In this example, the user account cloud service 410c is configured to record a list of users and to identify other data related to those users.

The user account cloud service 410c is shown with a network interface 1400, a communication manager 1402, server hardware 1404, and server system software 1406. In addition, the user account cloud service 410c is shown with a user identification module 1408, a purchase history module 1410, an engagement module 1412, and an application usage history module 1414.

The user identification module 1408 can include, or reference, data related to users of beds with associated data processing systems. For example, the users can include customers, owners, or other users registered with the user account cloud service 410c or another service. Each user can have, for example, a unique identifier, and user credentials, demographic information, or any other technologically appropriate information. Each user can also have user-inputted preferences pertaining to the user's bed system (e.g., firmness settings, heating/cooling settings, inclined and/or declined positions of different regions of the bed, etc.), ambient environment (e.g., lighting, temperature, etc.), and/or peripheral devices (e.g., turning on or off a television, coffee maker, security system, alarm clock, etc.).

The purchase history module 1410 can include, or reference, data related to purchases by users. For example, the purchase data can include a sale's contact information, billing information, and salesperson information that is associated with the user's purchase of the bed system. Additionally, an index or indexes stored by the user account cloud service 410c can identify users that are associated with a purchase of the bed system.

The engagement 1412 can track user interactions with the manufacturer, vendor, and/or manager of the bed and or cloud services. This engagement data can include communications (e.g., emails, service calls), data from sales (e.g., sales receipts, configuration logs), and social network interactions. The engagement data can also include servicing, maintenance, or replacements of components of the user's bed system.

The usage history module 1414 can contain data about user interactions with one or more applications and/or remote controls of a bed. For example, a monitoring and configuration application can be distributed to run on, for example, computing devices 412. The computing devices 412 can include a mobile phone, laptop, tablet, computer, smartphone, and/or wearable device of the user. The computing devices 412 can also include a central controller or hub device that can be used to control operations of the bed system and one or more peripheral devices. Moreover, the computing devices 412 can include a home automation device. The application that is presented to the user via the computing devices 412 can log and report user interactions for storage in the application usage history module 1414. Additionally, an index or indexes stored by the user account cloud service 410c can identify users that are associated with each log entry. User interactions that are stored in the application usage history module 1414 can optionally be used to determine or otherwise predict user preferences and/or settings for the user's bed and/or peripheral devices that can improve the user's overall sleep quality.

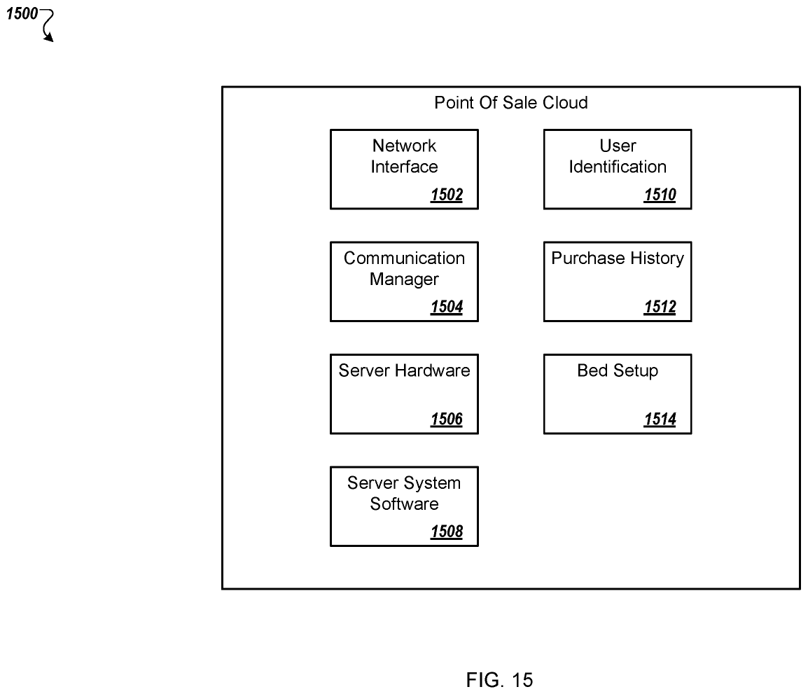

FIG. 15 is a block diagram of an example point of sale cloud service 1500 that can be used in a data processing system associated with a bed system, including those described above with respect to FIGS. 1-3. In this example, the point of sale cloud service 1500 is configured to record data related to users' purchases, specifically purchases of bed systems described herein.

The point of sale cloud service 1500 is shown with a network interface 1502, a communication manager 1504, server hardware 1506, and server system software 1508. In addition, the point of sale cloud service 1500 is shown with a user identification module 1510, a purchase history module 1512, and a bed setup module 1514.

The purchase history module 1512 can include, or reference, data related to purchases made by users identified in the user identification module 1510. The purchase information can include, for example, data of a sale, price, and location of sale, delivery address, and configuration options selected by the users at the time of sale. These configuration options can include selections made by the user about how they wish their newly purchased beds to be setup and can include, for example, expected sleep schedule, a listing of peripheral sensors and controllers that they have or will install, etc.

The bed setup module 1514 can include, or reference, data related to installations of beds that users purchase. The bed setup data can include, for example, a date and address to which a bed is delivered, a person who accepts delivery, configuration that is applied to the bed upon delivery (e.g., firmness settings), name or names of a user or users who will sleep on the bed, which side of the bed each user will use, etc.

Data recorded in the point of sale cloud service 1500 can be referenced by a user's bed system at later dates to control functionality of the bed system and/or to send control signals to peripheral components according to data recorded in the point of sale cloud service 1500. This can allow a salesperson to collect information from the user at the point of sale that later facilitates automation of the bed system. In some examples, some or all aspects of the bed system can be automated with little or no user-entered data required after the point of sale. In other examples, data recorded in the point of sale cloud service 1500 can be used in connection with a variety of additional data gathered from user-entered data.

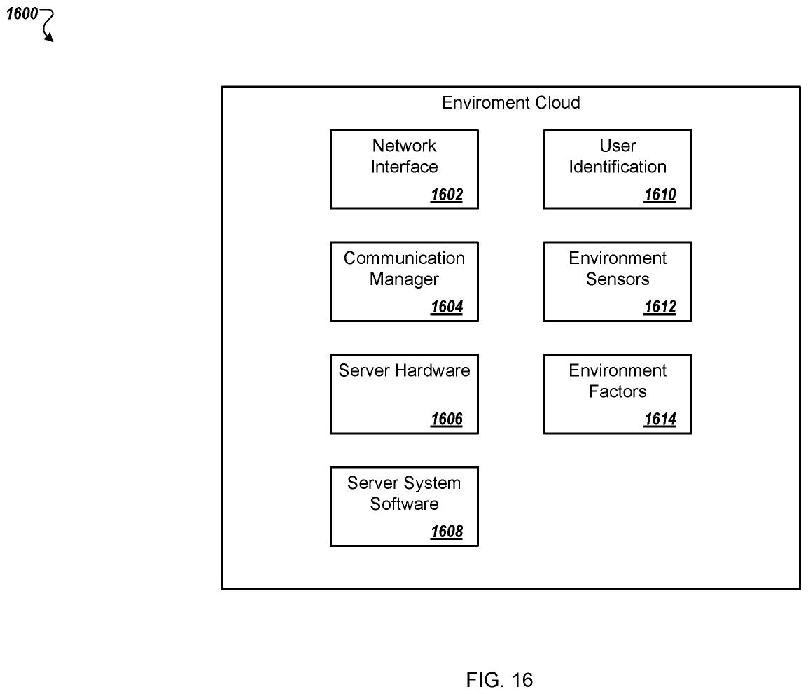

FIG. 16 is a block diagram of an example environment cloud service 1600 that can be used in a data processing system associated with a bed system, including those described above with respect to FIGS. 1-3. In this example, the environment cloud service 1600 is configured to record data related to users' home environment.

The environment cloud service 1600 is shown with a network interface 1602, a communication manager 1604, server hardware 1606, and server system software 1608. In addition, the environment cloud service 1600 is shown with a user identification module 1610, an environmental sensors module 1612, and an environmental factors module 1614.

The environmental sensors module 1612 can include a listing and identification of sensors that users identified in the user identification module 1610 have installed in and/or surrounding their bed. These sensors may include any sensors that can detect environmental variables, including but not limited to light sensors, noise/audio sensors, vibration sensors, thermostats, movement sensors (e.g., motion), etc. Additionally, the environmental sensors module 1612 can store historical readings or reports from those sensors. The environmental sensors module 1612 can then be accessed at a later time and used by one or more of the cloud services described herein to determine sleep quality and/or health information of the users.

The environmental factors module 1614 can include reports generated based on data in the environmental sensors module 1612. For example, the environmental factors module 1614 can generate and retain a report indicating frequency and duration of instances of increased lighting when the user is asleep based on light sensor data that is stored in the environment sensors module 1612.

In the examples discussed here, each cloud service 410 is shown with some of the same components. In various configurations, these same components can be partially or wholly shared between services, or they can be separate. In some configurations, each service can have separate copies of some or all of the components that are the same or different in some ways. Additionally, these components are only provided as illustrative examples. In other examples, each cloud service can have different number, types, and styles of components that are technically possible.

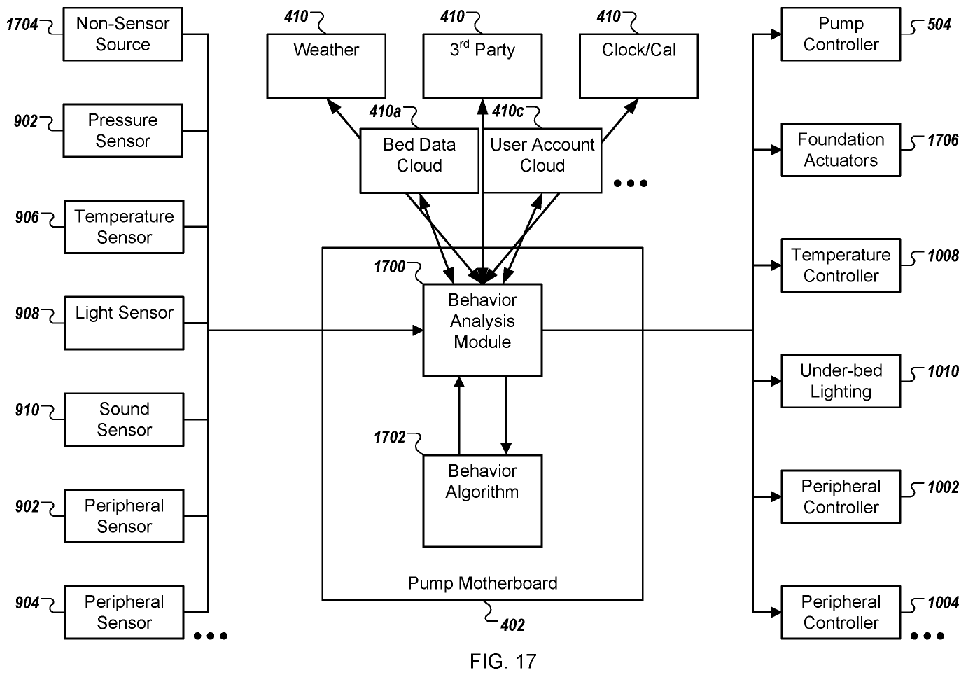

FIG. 17 is a block diagram of an example of using a data processing system associated with a bed (e.g., a bed of the bed systems described herein, such as in FIGS. 1-3) to automate peripherals around the bed. Shown here is a behavior analysis module 1700 that runs on the pump motherboard 402. For example, the behavior analysis module 1700 can be one or more software components stored on the computer memory 512 and executed by the processor 502.

In general, the behavior analysis module 1700 can collect data from a wide variety of sources (e.g., sensors 902, 904, 906, 908, and/or 910, non-sensor local sources 1704, cloud data services 410a and/or 410c) and use a behavioral algorithm 1702 (e.g., one or more machine learning models) to generate one or more actions to be taken (e.g., commands to send to peripheral controllers, data to send to cloud services, such as the bed data cloud 410a and/or the user account cloud 410c). This can be useful, for example, in tracking user behavior and automating devices in communication with the user's bed.

The behavior analysis module 1700 can collect data from any technologically appropriate source, for example, to gather data about features of a bed, the bed's environment, and/or the bed's users. Some such sources include any of the sensors of the sensor array 406 that is previously described (e.g., including but not limited to sensors such as 902, 904, 906, 908, and/or 910). For example, this data can provide the behavior analysis module 1700 with information about a current state of the environment around the bed. For example, the behavior analysis module 1700 can access readings from the pressure sensor 902 to determine the pressure of an air chamber in the bed. From this reading, and potentially other data, user presence in the bed can be determined. In another example, the behavior analysis module 1700 can access the light sensor 908 to detect the amount of light in the bed's environment. The behavior analysis module 1700 can also access the temperature sensor 906 to detect a temperature in the bed's environment and/or one or more microclimates in the bed. Using this data, the behavior analysis module 1700 can determine whether temperature adjustments should be made to the bed's environment and/or components of the bed in order to improve the user's sleep quality and overall comfortability.

Similarly, the behavior analysis module 1700 can access data from cloud services and use such data to make more accurate determinations of user sleep quality, health information, and/or control of the user's bed and/or peripheral devices. For example, the behavior analysis module 1700 can access the bed cloud service 410a to access historical sensor data 1212 and/or advanced sleep data 1214. Other cloud services 410, including those previously described can be accessed by the behavior analysis module 1700. For example, the behavior analysis module 1700 can access a weather reporting service, a 3^{rd} party data provider (e.g., traffic and news data, emergency broadcast data, user travel data), and/or a clock and calendar service. Using data that is retrieved from the cloud services 410, the behavior analysis module 1700 can more accurately determine user sleep quality, health information, and/or control of the user's bed and/or peripheral devices.

Similarly, the behavior analysis module 1700 can access data from non-sensor sources 1704. For example, the behavior analysis module 1700 can access a local clock and calendar service (e.g., a component of the motherboard 402 or of the processor 502). The behavior analysis module 1700 can use the local clock and/or calendar information to determine, for example, times of day that the user is in the bed, asleep, waking up, and/or going to bed.

The behavior analysis module 1700 can aggregate and prepare this data for use with one or more behavioral algorithms 1702. As mentioned, the behavioral algorithm 1702 can include machine learning models. The behavioral algorithms 1702 can be used to learn a user's behavior and/or to perform some action based on the state of the accessed data and/or the predicted user behavior. For example, the behavior algorithm 1702 can use available data (e.g., pressure sensor, non-sensor data, clock and calendar data) to create a model of when a user goes to bed every night. Later, the same or a different behavioral algorithm 1702 can be used to determine if an increase in air chamber pressure is likely to indicate a user going to bed and, if so, send some data to a third-party cloud service 410 and/or engage a peripheral controller 1002 or 1004, foundation actuators 1006, a temperature controller 1008, and/or an under-bed lighting controller 1010.

In the example shown, the behavioral analysis module 1700 and the behavioral algorithm 1702 are shown as components of the pump motherboard 402. However, other configurations are possible. For example, the same or a similar behavioral analysis module 1700 and/or behavioral algorithm 1702 can be run in one or more cloud services, and resulting output can be sent to the pump motherboard 402, a controller in the controller array 408, or to any other technologically appropriate recipient described throughout this document.

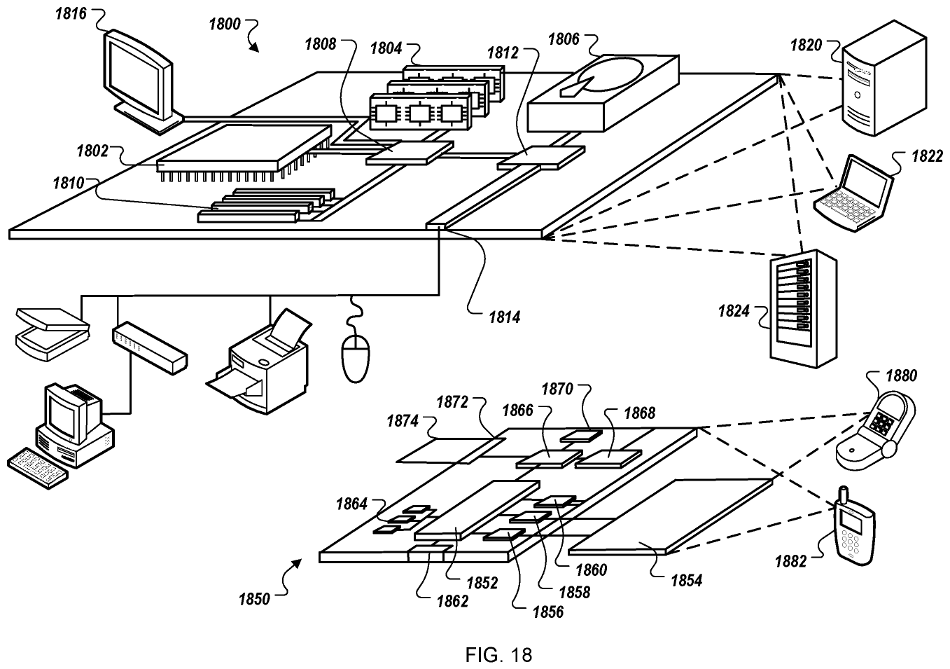

FIG. 18 shows an example of a computing device 1800 and an example of a mobile computing device that can be used to implement the techniques described here. The computing device 1800 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1800 includes a processor 1802, a memory 1804, a storage device 1806, a high-speed interface 1808 connecting to the memory 1804 and multiple high-speed expansion ports 1810, and a low-speed interface 1812 connecting to a low-speed expansion port 1814 and the storage device 1806. Each of the processor 1802, the memory 1804, the storage device 1806, the high-speed interface 1808, the high-speed expansion ports 1810, and the low-speed interface 1812, are interconnected using various busses, and can be mounted on a common motherboard or in other manners as appropriate. The processor 1802 can process instructions for execution within the computing device 1800, including instructions stored in the memory 1804 or on the storage device 1806 to display graphical information for a GUI on an external input/output device, such as a display 1816 coupled to the high-speed interface 1808. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 1804 stores information within the computing device 1800. In some implementations, the memory 1804 is a volatile memory unit or units. In some implementations, the memory 1804 is a non-volatile memory unit or units. The memory 1804 can also be another form of computer-readable medium, such as a magnetic or optical disk.

The storage device 1806 is capable of providing mass storage for the computing device 1800. In some implementations, the storage device 1806 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 1804, the storage device 1806, or memory on the processor 1802.

The high-speed interface 1808 manages bandwidth-intensive operations for the computing device 1800, while the low-speed interface 1812 manages lower bandwidth-intensive operations. Such allocation of functions is exemplary only. In some implementations, the high-speed interface 1808 is coupled to the memory 1804, the display 1816 (e.g., through a graphics processor or accelerator), and to the high-speed expansion ports 1810, which can accept various expansion cards (not shown). In the implementation, the low-speed interface 1812 is coupled to the storage device 1806 and the low-speed expansion port 1814. The low-speed expansion port 1814, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 1800 can be implemented in a number of different forms, as shown in the figure. For example, it can be implemented as a standard server 1820, or multiple times in a group of such servers. In addition, it can be implemented in a personal computer such as a laptop computer 1822. It can also be implemented as part of a rack server system 1824. Alternatively, components from the computing device 1800 can be combined with other components in a mobile device (not shown), such as a mobile computing device 1850. Each of such devices can contain one or more of the computing device 1800 and the mobile computing device 1850, and an entire system can be made up of multiple computing devices communicating with each other.

The mobile computing device 1850 includes a processor 1852, a memory 1864, an input/output device such as a display 1854, a communication interface 1866, and a transceiver 1868, among other components. The mobile computing device 1850 can also be provided with a storage device, such as a micro-drive or other device, to provide additional storage. Each of the processor 1852, the memory 1864, the display 1854, the communication interface 1866, and the transceiver 1868, are interconnected using various buses, and several of the components can be mounted on a common motherboard or in other manners as appropriate.

The processor 1852 can execute instructions within the mobile computing device 1850, including instructions stored in the memory 1864. The processor 1852 can be implemented as a chip set of chips that include separate and multiple analog and digital processors. The processor 1852 can provide, for example, for coordination of the other components of the mobile computing device 1850, such as control of user interfaces, applications run by the mobile computing device 1850, and wireless communication by the mobile computing device 1850.

The processor 1852 can communicate with a user through a control interface 1858 and a display interface 1856 coupled to the display 1854. The display 1854 can be, for example, a TFT (Thin-Film-Transistor Liquid Crystal Display) display or an OLED (Organic Light Emitting Diode) display, or other appropriate display technology. The display interface 1856 can comprise appropriate circuitry for driving the display 1854 to present graphical and other information to a user. The control interface 1858 can receive commands from a user and convert them for submission to the processor 1852. In addition, an external interface 1862 can provide communication with the processor 1852, so as to enable near area communication of the mobile computing device 1850 with other devices. The external interface 1862 can provide, for example, for wired communication in some implementations, or for wireless communication in other implementations, and multiple interfaces can also be used.

The memory 1864 stores information within the mobile computing device 1850. The memory 1864 can be implemented as one or more of a computer-readable medium or media, a volatile memory unit or units, or a non-volatile memory unit or units. An expansion memory 1874 can also be provided and connected to the mobile computing device 1850 through an expansion interface 1872, which can include, for example, a SIMM (Single In Line Memory Module) card interface. The expansion memory 1874 can provide extra storage space for the mobile computing device 1850, or can also store applications or other information for the mobile computing device 1850. Specifically, the expansion memory 1874 can include instructions to carry out or supplement the processes described above, and can include secure information also. Thus, for example, the expansion memory 1874 can be provide as a security module for the mobile computing device 1850, and can be programmed with instructions that permit secure use of the mobile computing device 1850. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 1864, the expansion memory 1874, or memory on the processor 1852. In some implementations, the computer program product can be received in a propagated signal, for example, over the transceiver 1868 or the external interface 1862.

The mobile computing device 1850 can communicate wirelessly through the communication interface 1866, which can include digital signal processing circuitry where necessary. The communication interface 1866 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 1868 using a radio-frequency. In addition, short-range communication can occur, such as using a Bluetooth, WiFi, or other such transceiver (not shown). In addition, a GPS (Global Positioning System) receiver module 1870 can provide additional navigation- and location-related wireless data to the mobile computing device 1850, which can be used as appropriate by applications running on the mobile computing device 1850.

The mobile computing device 1850 can also communicate audibly using an audio codec 1860, which can receive spoken information from a user and convert it to usable digital information. The audio codec 1860 can likewise generate audible sound for a user, such as through a speaker, e.g., in a handset of the mobile computing device 1850. Such sound can include sound from voice telephone calls, can include recorded sound (e.g., voice messages, music files, etc.) and can also include sound generated by applications operating on the mobile computing device 1850.

The mobile computing device 1850 can be implemented in a number of different forms, as shown in the figure. For example, it can be implemented as a cellular telephone 1880. It can also be implemented as part of a smart-phone 1882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

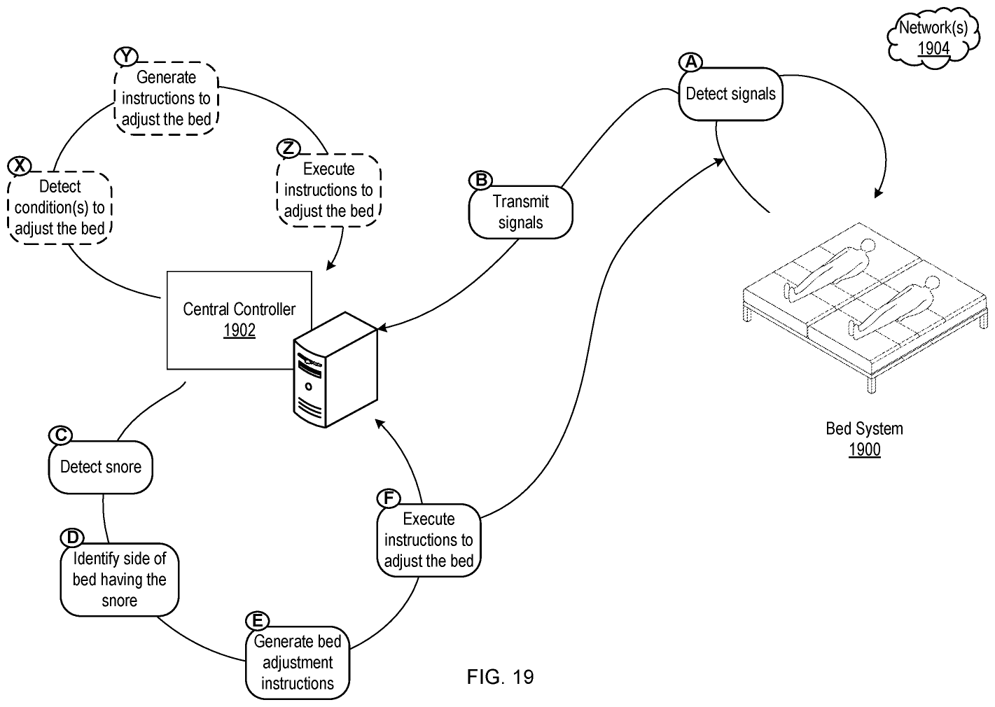

FIG. 19 is a conceptual diagram of a bed system 1900 environment for detecting and responding to snore. The bed system 1900 can include a central controller 1902. In some implementations, the central controller 1902 can be separate from the bed system 1900 and may be in communication with components of the bed system 1900 via network(s) 1904. For example, the central controller 1902 can be a cloud-based service, server, computing system, and/or device. The central controller 1902 can communicate with components of the bed system 1900 such as actuators and/or motors that can be used to mechanically adjust the bed system 1900 (e.g., incline sides of the bed system 1900).

In brief, the central controller 1902 can be configured to perform one or more operations for controlling/adjusting the bed system 1900. As described throughout this disclosure, the central controller 1902 can be used to adjust temperature and/or pressure of the bed system 1900. The central controller 1902 can also be used to adjust the bed system 1900, such as tilting and/or inclining one or more sides of the bed system 1900.

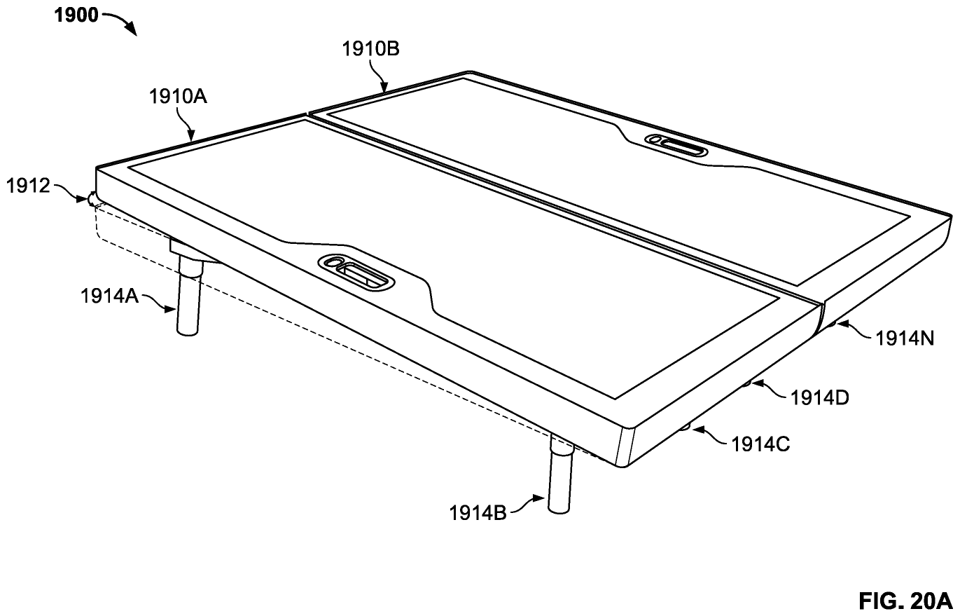

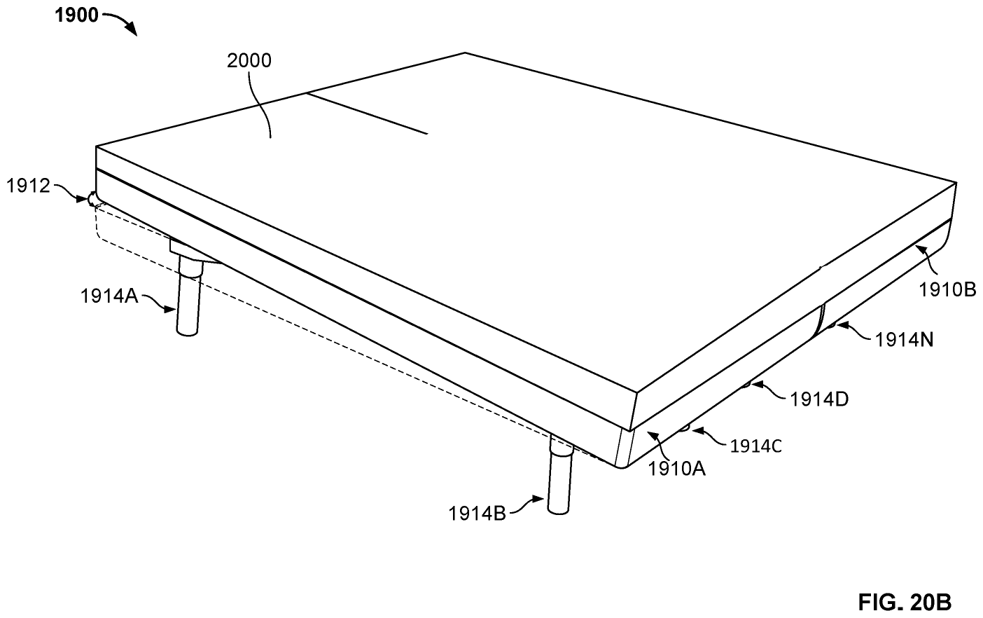

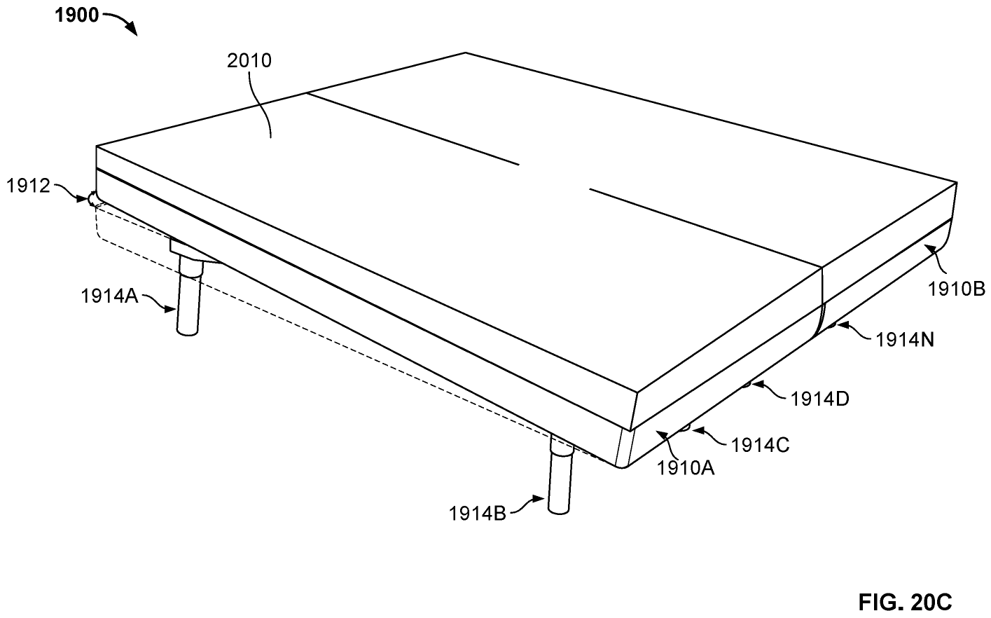

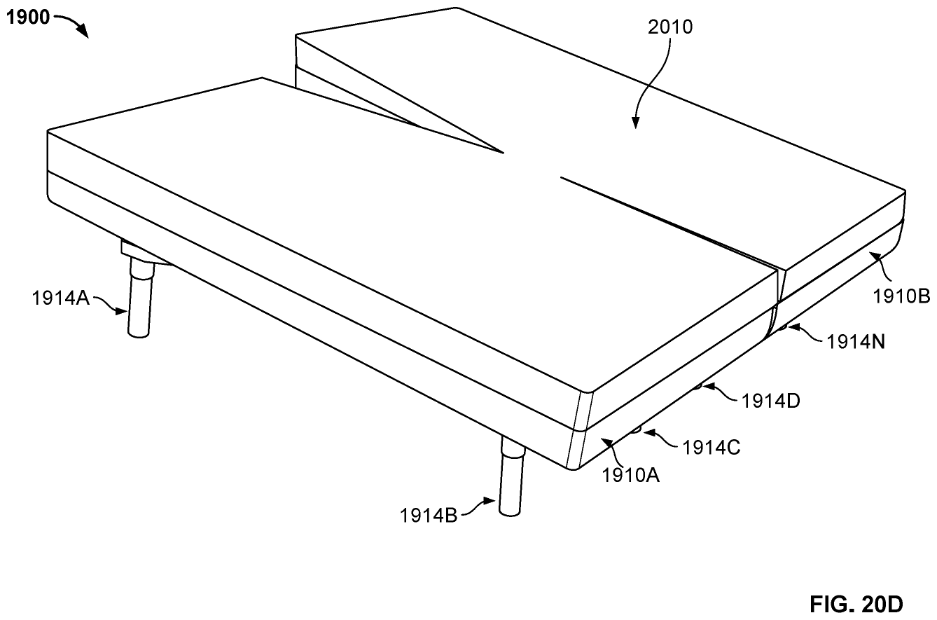

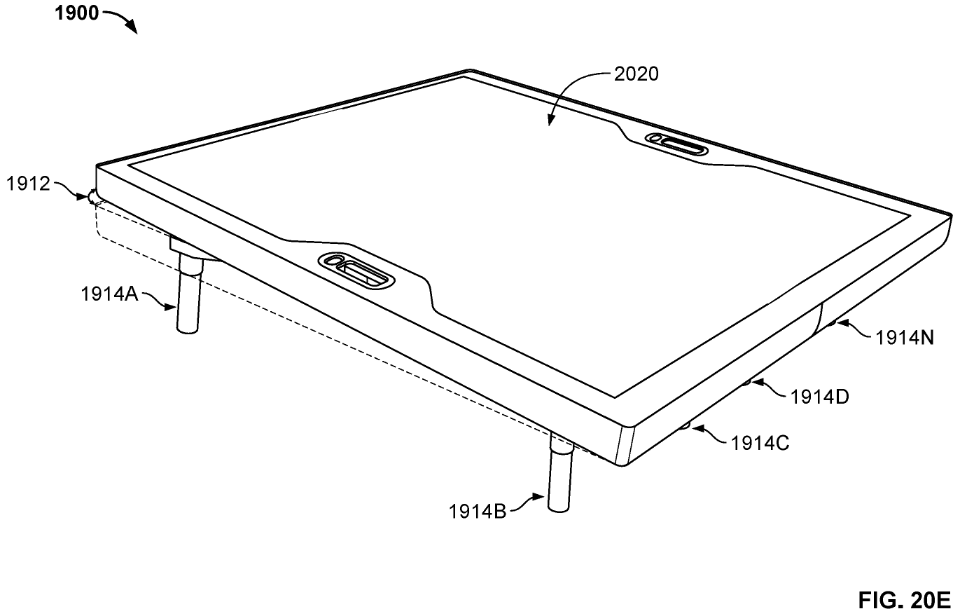

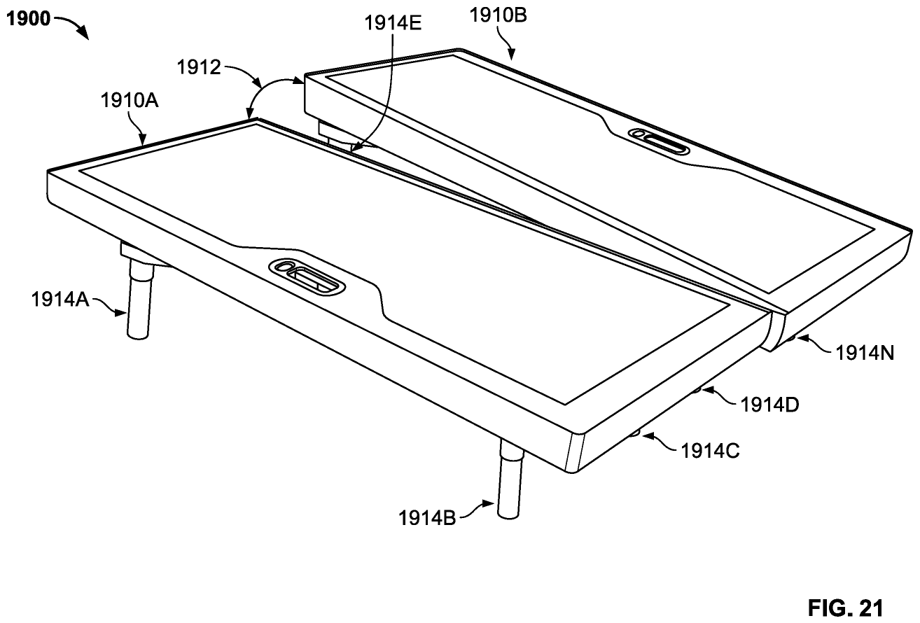

The bed system 1900, as described throughout this disclosure and further in FIGS. 20-21, can include a mattress and an adjustable foundation. The mattress can be an inflatable air mattress. The adjustable foundation can include one or more panels that can be uniformly (e.g., collectively) and/or separately articulated to one or more different positions. As described further below, the adjustable foundation can be articulated (e.g., tilted, inclined) to a position intended to reduce, mitigate, or otherwise prevent snoring. In this position, a head section of the adjustable foundation can be elevated (e.g., inclined or raised to a height) above a foot section of the adjustable foundation while maintaining the user's body in a plane. Moreover, in this position, the adjustable foundation can remain substantially straight.

The adjustable foundation can be rigid with a pivot (e.g. at a midpoint of the foundation or at other locations). A motor can move the bed around the pivot in order for the adjustable foundation to be tilted to the one or more positions, such as a position for snore mitigation, as described throughout this disclosure. The adjustable foundation may also be made of multiple panels, such as a head, back, and foot section. Each of those sections can be linked together and driven by a motor such that the entire foundation can be tilted to the one or more positions.

In some implementations, the bed system 1900 can be intended for two users. The adjustable foundation may include two sides (e.g., sections). A left side can support a first user resting on the left side of the bed system 1900 while a right side can support a second user resting on the right side of the bed system 1900. Each side of the adjustable foundation can include respective panels/sections, actuators, and/or motors. For example, the left side of the adjustable foundation can include a motor that is configured to tilt the entire left side into one or more positions. The right side of the adjustable foundation can also include a separate motor configured to work independently of the left side motor to tilt the right side of the adjustable foundation. As a result, a side of the bed system 1900 from which snoring originates can be automatically adjusted to mitigate the snoring without disrupting sleep of either user in the bed system 1900.

Still referring to FIG. 19, signals can be detected at the bed system 1900 (A). As described further below, the signals can include acoustic signals and/or pressure signals. The acoustic signals can be detected by one or more acoustic sensors that can be integrated into the bed system 1900. The acoustic sensors can also be separate from the bed system 1900 and in communication with components of the bed system 1900 and/or the central controller 1902 via the network(s) 1904. In some examples, the acoustic sensors can be microphones.

The pressure signals can be detected by one or more pressure sensors that can be integrated into the bed system 1900. For example, the pressure sensors can be configured to a pump of the bed system 1900. The pressure sensors can also be configured within air chambers of the inflatable air mattress. The pressure sensors may also be load sensors that are integrated into legs of the bed system 1900. One or more other pressure sensors can be used and placed in various locations of the bed system 1900, as described throughout this disclosure.

The detected signals can be transmitted to the central controller 1902 in B. Using the detected signals, the central controller 1902 can detect whether a user of the bed system 1900 is snoring (C). As described further below, the central controller 1902 can correlate the acoustic signals and the pressure signals using a machine learning model to determine whether the user is snoring. Such processing of the signals can occur during one or more predetermined periods of analysis. For example, this processing can occur within 20 seconds. This processing can also occur in one or more other periods of analysis, including but not limited to every 30 seconds, 40 seconds, 50 seconds, 1 minute, 2 minutes, etc. Furthermore, the processing of the signals described herein can occur in real-time.

The central controller 1902 may also identify a side of the bed system 1900 from which the detected snore originates in D. The side of the bed system 1900 having the snore can be identified using the correlated acoustic and pressure signals. In some implementations, the side of the bed system 1900 having the snore can be identified using just one of the acoustic signals or the pressure signals. For example, the central controller 1902 can determine ballistocardiograph (BCG) data and/or other biometric parameters from the pressure signals. The central controller 1902 can then determine whether the BCG data and/or other biometric parameters are indicative of snoring to determine which side of the bed from which the snoring originates.

As another example, the central controller 1902 can perform a plausibility check using the pressure signals to determine which side of the bed system 1900 from which the snoring originates. In some implementations, the central controller 1902 can detect snore in C, identify the side of the bed system 1900 having the snore in D, and then check the bed system 1900 a second time for snore as part of the plausibility check. The central controller 1902 can therefore assess acoustic and pressure signals within some predetermined period of time (e.g., 30 seconds, 1 minute, 2 minutes, etc.) do determine that the user is snoring, the side of the bed system 1900 from which the snoring originates, and an adjustment to the bed system 1900 to respond to the snoring.

In some implementations, the plausibility check may also be performed to determine whether the user has stopped snoring and/or started snoring again. If, for example, it is determined that the user has stopped snoring, then the central controller 1902 may generate instructions that cause the bed system 1900 to be lowered by a predetermined amount of degrees. If it is determined that the user starts snoring again, then the central controller 1902 can generate instructions that cause the bed system 1900 to be raised again to a particular position intended for stopping snoring. The purpose of such minor adjustments can be to unblock soft tissues at the back of the user's throat that cause the snoring without disrupting the user's sleep or comfortability during sleep.

Based on determining that the user is snoring and the side of the bed system 1900 from which the snore originates, the central controller 1902 can generate bed adjustment instructions (E). As described herein, the instructions can cause the side of the bed 1900 having the snore to be tilted/inclined into a position that is intended to reduce, mitigate, or otherwise prevent the user's snoring. In this position, the user's head is elevated (e.g., inclined/raised) above the user's feet while maintaining the user's body along a plane.

This position can maintain the bed system 1900 at a 7° angle relative to a ground surface such that a head section of the bed system 1900 is elevated above a foot section of the bed system 1900 along a plane. An entire deck of the bed system 1900 can therefore be inclined/tilted to a position that reduces or stops snoring and maintains comfortability of the user while they are sleeping. Therefore, at the 7° angle, the user may not slide down the bed system 1900, nor may the user wake up from soreness in their neck, back, or other parts of the body. In some implementations, the bed system 1900 can be adjusted to one or more other preferred angles relative to the ground surface that are intended to reduce or stop snore and maintain the user's comfortability while sleeping.

More particularly, the instructions, when executed by an actuation system of the bed system 1900, can cause one or more motors to operate and thus tilt (e.g., incline) the side of the bed system 1900 to the desired position (e.g., a position intended to mitigate snore). Accordingly, the central controller 1902 can execute the instructions to adjust the bed system 1900 in F.

Optionally, the central controller 1902 may also perform X-Z. X-Z can be performed at any time while the bed system 1900 is occupied. X-Z can be performed, in some implementations, after the bed system 1900 has been adjusted using the C-F. For example, X-Z can be performed to adjust the bed system 1900 a second time after the bed system 1900 has been adjusted to the position intended to stop the user's snore.

In X, the central controller 1902 can detect one or more conditions that suggest the bed system 1900 should be adjusted again. The condition, for example, can be an amount of time that has passed since the bed system 1900 was adjusted to the position for snore mitigation in F. If the amount of time exceeds some threshold period of time (e.g., an expected length of time for the user's sleep cycle), then the condition can be satisfied and the central controller 1902 can proceed to Y. As another example, the condition can be the user waking up. Therefore, if one or more sensors at the bed system detect activity by the user that, when analyzed by the central controller 1902, indicates that the user is waking up or has woken up, then the condition can be satisfied and the central controller 1902 can proceed to Y. As yet another example, the condition can be receiving user input indicating selection of an option to move the bed system 1900 into another position. Thus, if the user provides input via a remote control of the bed system 1900 and/or an application in a mobile phone that is in communication with the central controller 1902, where that input indicates selection of another position for the bed system 1900, the condition can be satisfies and the central controller 1902 can proceed to Y. One or more other conditions can be defined, detected, and/or determined in the optional X.

In the optional Y, the central controller 1902 can generate instructions to adjust the bed system 1900. These instructions can be based on the condition(s) of X. For example, if the user's sleep cycle has ended and/or the user has woken up, the central controller 1902 can generate instructions that, when executed by the actuation system of the bed system 1900, can cause the motors to adjust the bed system 1900 to a flat position. In the flat position, the user's head and feet may be parallel with each other on a horizontal plane. As another example, if the user provided user input selecting another position, the central controller 1902 can generate instructions that, when executed by the actuation system, can cause the motors to adjust the bed system 1900 to the user-desired position.

Otherwise, if the condition(s) is not detected/satisfied in X, the bed system 1900 can remain inclined in the position for snore mitigation. This can be advantageous to ensure the user's safety. After all, if the bed system 1900 automatically adjusts while the user is still sleeping, the user's arms, hands, or other body parts may get stuck between moving components of the bed system 1900, thereby causing injury. As another example, if the bed system 1900 is automatically adjusting while the user is still sleeping, the automatic adjustment can cause potential injury to pets or other animals in the user's sleep environment.

Once the instructions are generated in Y, the central controller 1902 can execute the instructions to adjust the bed system 1900 (Z). As described in reference to F, the central controller 1902 can transmit the instructions to the actuation system for execution. The central controller 1902 may also execute the instructions to cause the motors of the bed system 1900 to adjust accordingly.

Although the bed system 1900 is shown as supporting two users, A-F and/or X-Z can also be performed for a bed system that is intended for a single user.

FIGS. 20A-E show an example bed system 1900 inclined to various positions for mitigating detected snore. In FIG. 20A, the bed system 1900 is an adjustable foundation. The bed system 1900 can be used by two sleepers (e.g., users). The bed system 1900 can include two sections, a first foundation section 1910A and a second foundation section 1910B. Each of the foundation sections 1910A and 1910B can support a section of a mattress on which a sleeper rests. For example, a first sleeper can sleep on the first foundation section 1910A and a second sleeper can sleep on the second foundation section 1910B.

Each foundation section 1910A and 1910B can be independently articulable and controllable by the central controller 1902 described in reference to FIG. 19. Each foundation section 1910A and 1910B can include respective openings for moving and/or routing wires for any components described throughout this disclosure. The openings can also be configured to receive air hoses of an inflatable air mattress or other component of the bed system 1900 described throughout this disclosure.

The foundation sections 1910A and 1910B can be configured to adjust in overall height and/or inclination. Adjusting the overall height of either of the foundation sections 910A or 910B can beneficially provide easy bed entry and exit. As described throughout this disclosure, the central controller 1902 can generate and send instructions to an actuation system of the bed system 1900 that causes motors of one of the foundation sections 1910A and 1910B to raise or lower respective legs 1914A-N of the foundation sections 1910A and 1910B and/or tilt the respective foundation section 1910A and/or 1910B.

For example, as shown in FIG. 20A, the actuation system of the bed system 1900 can be activated to extend the respective legs 1914A-N of the foundation sections 1910A and 1910B to raise a height of head portions of the foundation sections 1910A and 1910B to a predetermined height. The respective legs 1914A-N near foot portions of the foundation sections 1910A and 1910B can remain at a current height and thus may not be adjusted (or they can be adjusted to a lower height, in some implementations). As a result, the foundation sections 1910A and 1910B are tilted at an angle 1912 such that the head portions of the foundation sections 1910A and 1910B are elevated from the foot portions of the foundation sections 1910A and 1910B along a plane. By tilting the foundation sections 1910A and 1910B at the angle 1912, the user(s) can experience improved breathing, increased blood flow, improved circulation, and reduce or no snoring.

The angle 1912 can be 7° relative to a ground surface that is a horizontal plane. The foundation sections 1910A and/or 1910B can be inclined at the angle 1912 relative to horizontal, as shown in FIG. 20A. The angle 1912 can be determined based on a height of the head portions of the foundation sections 1910A and 1910B that is likely to reduce or stop snoring, improve circulation and blood flow, and maintain comfortability of the user while the user is sleeping. Therefore, the angle 1912 can be selected to reduce or eliminate vibration of soft tissue within the mouth or throat of the user in order to reduce or eliminate snoring by the user. As additional examples, the angle 1912 can be from about 5° to about 15° from horizontal. One or more other angles may also be determined and used with the disclosed techniques.

The techniques described herein for adjusting the bed system 1900 can be used with different foundation types as well as different mattress types. For example, various embodiments of the bed system 1900 can support a twin, full, queen, and/or regular king size mattress. When the foundation sections 1910A and 1910B are both tilted, the entire mattress placed on top of the foundation sections 1910A and 1910B can be tilted. The bed system 1900 can also support other types of mattresses, such as split top kings, split kings, and H-bed king mattresses. With these mattresses, when the foundation sections 1910A and 1910B are individually tilted, a respective side of the mattress can similarly be tilted. Alternatively, partially or entirely split mattresses (e.g. split top kings, split kings, and H-bed king mattresses) the entire mattress (or mattresses) placed on top of the foundation sections 1910A and 1910B can be tilted.

FIG. 20B depicts the example bed system 1900 with a split top king mattress 2000. As shown, when both foundation sections 1910A and 1910B are tilted to a desired position for mitigating detected snore, the entire split top king mattress 2000 is also tilted.

FIG. 20C depicts the example bed system 1900 with an H-bed king mattress 2010. The H-bed king mattress can be substantially H-shaped, with a split at a head of the mattress, a split at a foot of the mattress, and a joined middle portion of the mattress. As shown, when both foundation sections 1910A and 1910B are tilted to a desired position for mitigating detected snore, the entire H-bed king mattress 2010 can be tilted.

In some implementations, as shown in FIG. 20D, the foundation sections 1910A and 1910B can be separately and individually controlled to be tilted while the H-bed king mattress 2010 is supported thereon. In the example of FIG. 20D, when the foundation section 1910B is tilted such that a head portion of the foundation section 1910B is raised and a foot section of the foundation section 1910B is lowered, and the foundation section 1910A remains in a relatively horizontal/flat position, a right side of the H-bed king mattress 2010 that is supported by the foundation section 1910B is also tilted. A left side of the H-bed king mattress 2010 that is supported by the foundation section 1910A can remain in a relatively horizontal/flat position, even though there may be some tension along a pivot point at a midpoint of the H-bed king mattress 2010. The pivot point of the H-bed king mattress 2010 can remain relatively stationary when the mattress 2010 is tilted in response to the foundation section 1910B being individually tilted.

FIG. 20E depicts the bed system 1900 having a unitary foundation section 2020. As a result, the unitary foundation section 2020 can be tilted as described throughout this disclosure. Tilting the unitary foundation section 2020 can cause an entire mattress supported by the bed system 1900 to also tilt. The unitary foundation section 2020 can support different mattress types, including but not limited to twin, full, queen, and/or regular king size mattresses.

FIG. 21 shows the example bed system 1900 in which one side of the bed system 1900 is inclined to a position for mitigating detected snore. Each of the foundation sections 1910A and 1910B can be individually controlled and actuated by the central controller 1902. As described throughout this disclosure, the central controller 1902 can determine which of the foundation sections 1910A and 1910B to incline based on detecting snore and from which side of the foundation 1900 the snore originates.

In the example of FIG. 21, the foundation section 1910B is elevated or tilted (e.g., inclined) to the position for snore mitigation, which is described throughout this disclosure. The central controller 1902 might have determined that snore was detected in the bed system 1900 and the snore was detected at the foundation section 1910B. The foundation section 1910A remains in a flat (e.g., neutral) position in which a height of the respective legs 1914A-N supporting the foundation section 1910A remain at a neutral or base height. In other words, the foundation section 1910A is in a flat configuration with head and foot portions being in a horizontal or substantially horizontal orientation (e.g., relative to the ground surface).

The respective legs 1914A-N supporting the foundation section 1910B, on the other hand, can be raised in height by the actuation system of the bed system 1900 near the head portion of the foundation section 1910B to cause the head portion of the foundation section 1910B to be elevated with respect to the foot portion of the foundation section 1910B on the plane. Therefore, the foundation section 1910B can be inclined by the predetermined angle 1912.

Although FIGS. 20-21 are described in reference to the bed system 1900 that includes the first and second foundation sections 1910A and 1910B, the disclosure of FIGS. 20-21 can also apply to bed systems having one foundation section, such as just the first foundation section 1910A, which can be intended for one user.

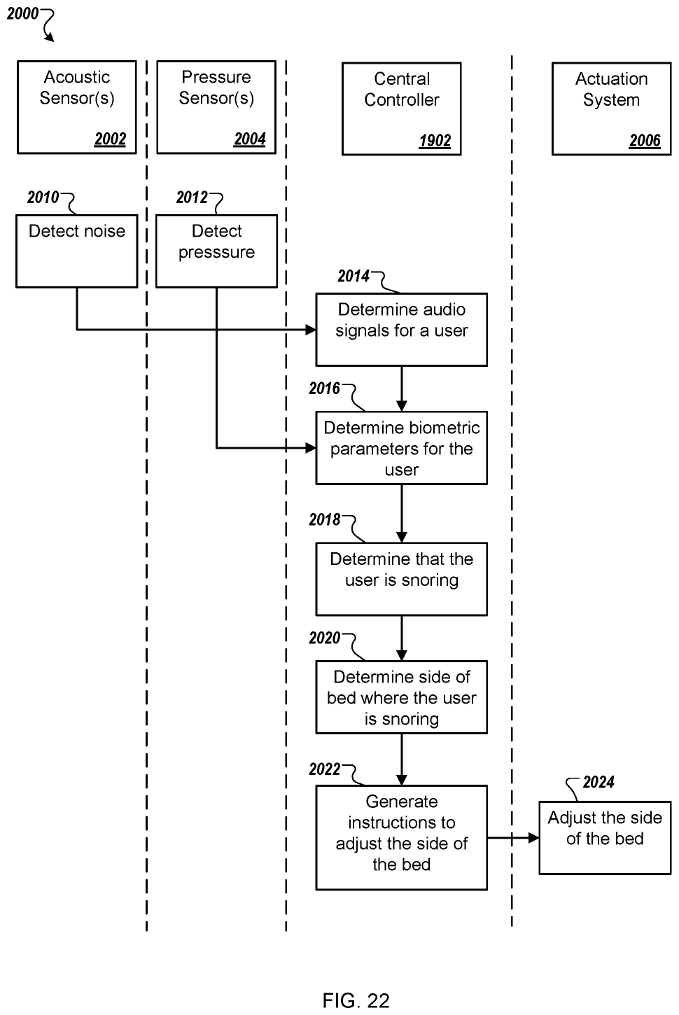

FIG. 22 is a swimlane diagram of an example process 2000 for detecting and responding to snore. The process 2000 can be performed to determine whether a user is snoring based on correlating audio and pressure signals detected at a bed system. Once it is determined by the components described herein that the user is snoring, instructions can be generated to cause an actuation system of the bed system to adjust the bed to a position intended to reduce or otherwise stop the snoring. In this position, the user's head is elevated above the user's feet along a substantially straight plane.

For clarity, the process 2000 is being described with reference to components of the bed system 1900, including but not limited to acoustic sensor(s) 2002, pressure sensor(s) 2004, the central controller 1902, and actuation system 2006. However, other system or systems can be used to perform the same or a similar process.

Referring to the process 2000 in FIG. 22, the process 2000 can begin when noise is detected by the acoustic sensor(s) 2002 (block 2010). The acoustic sensor(s) 2002 can sense audio at the bed system. In some implementations, the acoustic sensors 2002 can be located on an outer edge of a deck panel within 2 feet of an edge of the deck panel at a head of the bed. In some implementations, the acoustic sensors 2002 can be microphones that are part of the bed, positioned in a sleep environment of the user, and/or part of a user device, such as a mobile phone, smartphone, laptop, tablet, and/or home automation device. A microphone of a smartphone can, for example, detect noise at the bed and transmit the detected noise to the central controller 1902 for processing and analysis. When a user device detects the noise, the user device can also transmit an audio fingerprint to the central controller 1902. The audio fingerprint can be used by the central controller 1902 to calibrate and configure the central controller 1902 to detect snore based on that particular user device.

Before, during, and/or after block 2010, the pressure sensor(s) 2004 can detect pressure on or around a portion of the bed system (block 2012). The pressure sensor(s) 2004 can sense pressure on a mattress of the bed system. As described herein, the pressure sensor(s) 2004 can be part of the bed system.

Sometimes, one or more of the pressure sensor(s) 2004 can be positioned in support legs of the adjustable foundation of the bed system. These pressure sensor(s) 2004 can be load cells and can be configured to sense pressure on the bed system that can indicate a location of the user on the mattress. Moreover, in some implementations with beds designed for two simultaneous users laying side-by-side, a pressure in a first air chamber of the two sleeper bed can be monitored. A pressure in a second air chamber of the two sleeper bed can also be monitored. A first set of force sensors mounted to a first set of legs on the bed can be monitored. The first set of legs can be on a first side of the bed that includes the first air chamber. A second set of force sensors mounted to a second set of legs on the bed can be monitored. The second set of legs can be on a second side of the bed that includes the second air chamber. The pressure in the first air chamber can then be compared to a combined load of the first set of force sensors. The pressure in the second air chamber can be compared to a combined load of the second set of force sensors. A detected pressure in either the first air chamber or the second air chamber and a detected load on the corresponding force sensors can be associated with a presence of a user, which can be used to determine which side of the bed that snoring originates.

The bed system can also include a pump that can be operably connected to the mattress (e.g., an inflatable air mattress). The pressure sensor(s) 2004 can include a pressure transducer that can be positioned in the pump and fluidically connected to both a manifold of the pump and to the mattress to detect pressure signals of the bed system.

Sometimes, at least one load stream from at least one load sensor (e.g., pressure sensor) can be detected in block 2012. The at least one load stream represents a load sensed by the load sensor. In two sleeper bed implementations, a pressure stream from a first pressure sensor and a load stream of a first set of load sensors can be joined to create a first-side joined stream. Similarly, a pressure stream from a second pressure sensor and a load stream of a second set of load sensors can be joined to create a second-side joined stream. Such streams can be processed simultaneously or serially by the central controller 1902 to similar effects as in implementations with a single stream.

The detected noise can be transmitted to the central controller 1902 at a same or different time as the detected pressure is transmitted to the central controller 1902. For example, the detected noise can first be transmitted to the central controller 1902 and once the central controller 1902 processes the detected noise through blocks 2014 and 2018, the central controller 1902 can poll the pressure sensor(s) 2004 for any detected pressure. As described throughout this disclosure, polling the pressure sensor(s) 2004 for the detected pressure can be part of a plausibility check to determine from which side of the bed system the snoring originates and whether the user is in fact snoring. The central controller 1902 can poll the pressure sensor(s) 2004 at one or more time intervals after receiving and/or processing the detected noise from the acoustic sensor(s) 2002. The time intervals can include but are not limited to 30 seconds, 1 minute, 2 minutes, 3 minutes, etc.

Upon receiving the detected noise from the acoustic sensor(s) 2002, the central controller 1902 can determine audio signals for the user of the bed system (block 2014). For example, the noise can be determined to be a snore based on a signal from the one or more acoustic sensors 2002 by the central controller 1902.

Upon receiving the detected pressure from the pressure sensor(s) 2004, the central controller 1902 can determine biometric parameters for the user (block 2016). The biometric parameters can include BCG signals. As described throughout this disclosure, the biometric parameters can be determined using a machine learning model that was trained using a training dataset to detect and identify movements due to snore, which are reflected in pressure signals. The machine learning model can also be trained to detect and identify these movements, which can also be present in a frequency band of the audio signal.

For example, a sleeping parameter of the user can be generated from any of the detected pressure (e.g., at least a pressure stream and a load stream). In two sleeper bed implementations described above, the first-side joined stream and the second-side joined streams can be used to generate a first-side sleep parameter of the first user and a second-side sleep parameter of the second user. In some implementations, the sleeping parameter can include a restlessness of a user. In some implantations, a comparison parameter can be created by comparing the load stream and the pressure stream. Moreover, one or more other biometric parameters can be determined in block 2016.

Next, the central controller 1902 can determine whether the user is snoring in block 2018. The central controller 1902 can determine whether the user is snoring based off one of the audio signals and the biometric parameters. For example, the central controller 1902 can determine whether the user is snoring based on sound waves generated by the user and detected by the pressure sensor(s) 2004.

The central controller 1902 can also determine whether the user is snoring based on correlating the audio signals with the biometric parameters. The audio signals and the biometric parameters can be correlated using a machine learning model. The machine learning model can be previously trained to detect snore using a dataset of training acoustic data that was annotated with pressure signals when the adjustable foundation was inclined at one or more different angles. One or more of the audio signals and the biometric parameters can be provide as input to the machine learning model during runtime. The machine learning model can output an indication of whether snore is detected at the bed.

The central controller 1902 can also determine a side of the bed where the user is snoring (block 2020). For example, the central controller 1902 can analyze the biometric parameters to determine a left side or a right side of the mattress from which the snoring originates. The central controller 1902 can also correlate the biometric parameters with the audio signals to determine the side of the bed where the user is snoring.

If and when the bed is adjusted (e.g., inclined, tilted), it may be challenging for the pressure sensor(s) 2004 to accurately detect pressure signals, thereby making it more challenging to detect which side of the bed from which the snoring originates. The central controller 1902 can therefore apply a compensation to the detected pressure signals so that the pressure signals can be used to determine the side of the bed from which the snoring originates. The compensation can be offsets and/or scaling of the pressure signals so that the pressure signal can resemble what signals would be detected if the bed is not inclined, tilted, or otherwise adjusted. Typically, when the bed is adjusted, the pressure signals from the bed may increase by a certain delta pressure. After all, a decrease in volume (e.g., adjusting the bed) can cause an increase in pressure, especially when temperature is constant. Therefore, compensation can include subtracting the delta pressure from the pressure signals that are detected after the bed has been adjusted.

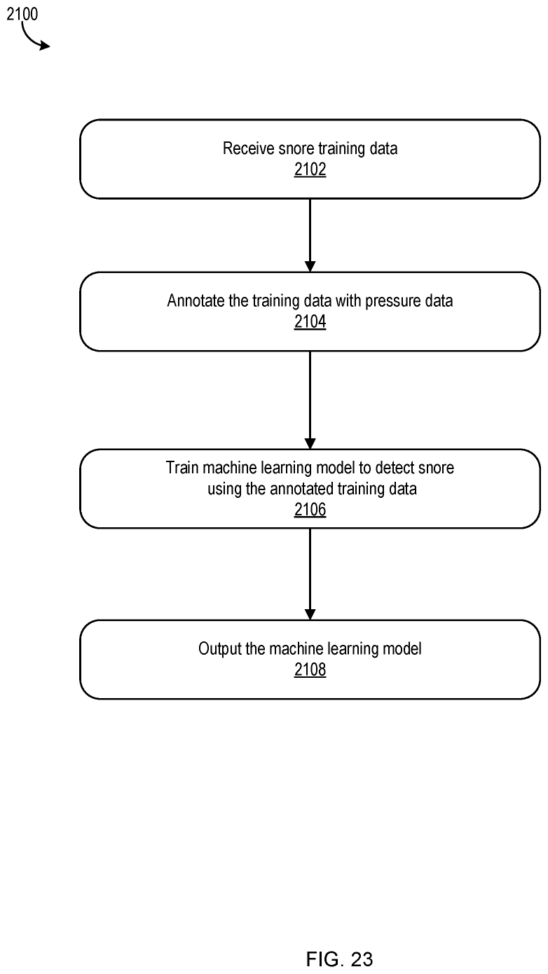

In some implementations, one or more machine learning models can be used to determine from which side of the bed the snoring originates. A machine learning model, for example, can be agnostic to an angle of inclination and thus can be used to accurately determine the side of the bed from which the snoring originates, regardless of the inclination of the bed when the pressure signals are detected. During training, as described further in reference to FIG. 23, the model can be trained with a training dataset of acoustic data that was captured/annotated with pressure signals when a bed was inclined at various different angles. As a result, the machine learning model can be used during runtime to accurately determine the side of the bed from which the snoring originates with the audio signals, regardless of a fidelity of detected pressure signals.

In implementations where the bed system is intended for a single user, the block 2020 may not be performed in the process 2000. In some cases, the block 2018 and 2020 can be performed simultaneously as part of a single determination based on the audio signals and biometric parameters.

In block 2022, the central controller 1902 can generate instructions to adjust the side of the bed. The instructions can be transmitted to the actuation system 2006 (e.g., articulation system) and used to tilt the adjustable foundation of the bed system to a predetermined position such that an angle between a head portion of the adjustable foundation and the ground is greater than an angle between a foot portion of the adjustable foundation and the ground, the ground being a horizontal plane.

The actuation system 2006 can adjust the side of the bed based on executing the instructions in block 2024. For example, the actuation system 2006 can activate, in response to receiving the instructions from the central controller 1902, one or more motors of the side of the bed to raise legs supporting a head portion of the side of the bed to a height that corresponds to an angle between the head portion of the side of the bed and the ground when the side of the bed is in a position for snore mitigation. As described herein, in this position, the user's head can be elevated (e.g., inclined, raised) above the user's feet while maintaining the body in a substantially straight plane. The angle, as described herein, can be 7°. As another example, the actuation system 2006 can activate, in response to receiving the instructions from the central controller 1902, one or more motors of the side of the bed to lower legs supporting a foot portion of the side of the bed to a height that corresponds to an angle between the foot portion of the side of the bed and the ground when the side of the bed is in the position for snore mitigation. For example, the angle can be less than 7°. In some implementations, the angle can be 7° or any other angle that is determined to reduce or otherwise stop snoring.

The actuation system 2006 may maintain the bed in the adjusted position based on one or more factors. For example, the bed can be maintained in the adjusted position until the user wakes up. The bed can also be maintained in the adjusted position until a certain amount of time passes since adjusting the bed and/or the user's sleep cycle ends (e.g., the bed can be adjusted to the position for snore mitigation for one cycle of the user's sleep then adjusted to a flat position at the end of the cycle). Leaving the bed in the adjusted position can be advantageous for a variety of reasons. For example, remaining in the adjusted position can prevent the user from snoring again. Remaining in the adjusted position can also prevent potential injury or other harm to the user or another user or animal if they get in the way of components of the bed when the bed is being automatically adjusted. Remaining in the adjusted position can also be advantageous to limit an amount of noise that may result from automatically adjusting the bed, thereby avoiding disturbing the user and a partner while they are sleeping in the bed.

In some implementations, the central controller 1902 can, in response to determining that the user has woken up, generate and transmit instructions to the actuation system 2006 to move the side of the bed into a neutral position (blocks 2022-2024). The actuation system 2006 can then lower, in response to receiving the instructions, the legs supporting the head portion of the side of the bed to a second height that corresponds to the neutral position. In the neutral position, the side of the bed can be in a relatively horizontal configuration in which the entire side of the bed is parallel with the ground. Sometimes, the actuation system 2006 may also raise, in response to receiving the instructions, the legs supporting the foot portion of the side of the bed to the second height that corresponds to the neutral position.

In some implementations, the actuation system 2006 can actuate one or more motors to adjust, based on receiving an indication of user input from a user device indicating selection of an option to move the side of the bed to another position, the height of one or more of the legs supporting the side of the bed to move the adjustable foundation to the another position. The user device can be any type of mobile device, remote control, or other computing device that may communicate with components of the bed system described herein.

The side of the bed can remain inclined until some user input is received indicating that the bed should be moved to another position (such as down into a flat or neutral position), as described throughout this disclosure. In some implementations, however, the side of the bed can optionally be moved to another position based on one or more other conditions. As an illustrative example, in some implementations, the side of the bed can remain inclined in the position for snore mitigation until the central controller 1902 determines that a sleep cycle of the user has ended. The central controller 1902 can make this determination based on continuously performing any one or more of the blocks 2010-2018. In response to determining that the sleep cycle of the user has ended, the central controller 1902 can generate instructions that, when executed by the actuation system 2006, cause one or more motors to be actuated to lower the side of the bed to a flat or neutral position. Sometimes, the central controller 1902 can control actuation of the one or more motors without sending instructions to the actuation system 2006.

As another illustrative example, in some implementations, the side of the bed can remain inclined in the position for snore mitigation for a predetermined amount of time. In response to determining that the predetermined amount of time has ended since the side of the bed was first inclined, the central controller 1902 can generate instructions that, when executed by the actuation system 2006, cause one or more motors to be actuated to lower the side of the bed to a flat or neutral position. Sometimes, the central controller 1902 can control actuation of the one or more motors without sending instructions to the actuation system 2006.

In some implementations, the bed system can include multiple air chambers, such as first and second air chambers. A first pressure sensor 2004 can be configured to sense air pressure of the first air chamber and a second pressure sensor 2004 can be configured to sense air pressure of the second air chamber. A first user can rest on top of the first air chamber and a second user can rest on top of the second air chamber. The central controller 1902 can determine biometric parameters for the first user supported by the first air chamber based on detected pressure by the first pressure sensor 2004. The central controller 1902 can determine biometric parameters for the second user supported by the second air chamber based on detected pressure by the second pressure sensor 2004. Based on analysis of the biometric parameters for the first and second users, the central controller 1902 can determine which of the user sis snoring. The central controller 1902 can also generate instructions that, when executed by the actuation system 2006, can cause one or more motors to articulate a side of the bed system where the first or second user is snoring.

FIG. 23 is a flowchart of a process 2100 for training a machine learning model to detect snore. One or more machine learning techniques can be used in the process 2100, such as a deep neural network (DNN), convolutional neural network (CNN), recurrent neural network, long short-term memory-LSTM, and/or other artificial neural networks or types of deep learning. The machine learning model can be trained using data from one or more users. As a result, the model can be applied to any bed system during runtime. In some implementations, the model can be trained using data specific to a particular user. As a result, the model can be user-specific, and each user of bed systems described herein can have their respective machine learning model(s). In yet some implementations, models can be trained based on clusters of users, where the clusters can be determined based on a variety of factors/characteristics of the users (e.g., age, demographics, geographic region, health parameters, etc.). An example user cluster can be for sleep apnea patients who may have a different spectral distribution in their snore audio signals in comparison to users who do not have sleep apnea.

The process 2100 can be performed by the central controller 1902. The process 2100 can also be performed by one or more other controllers, computing systems, devices, network of devices and/or systems, and/or cloud-based service/system. For example, the process 2100 for training the machine learning model can be performed by a computing system that is separate and remote form the central controller 1902 and/or the bed system 1900 or other bed systems. For illustrative purposes, the process 2100 is described from the perspective of a computer system.

Referring to the process 2100, the computer system can receive snore training data in block 2102. This training data can include acoustic signals that have been positively identified as indicative of snoring. The acoustic signals can be received from various different bed systems and can represent various different sounds that have been positively identified as snores. Moreover, these acoustic signals that positively identify snoring can be detected from bed systems that are inclined at many different angles. In some implementations, the computer system can positively identify the snoring in the acoustic signals. Sometimes, a relevant user can positively identify the snoring. Similarly, the snore training data can be annotated and labeled then received in block 2102. Annotating and labeling can be performed automatically by the computer system and/or manually by the relevant user. When annotated and labeled training data is received in block 2102, the computer system can skip block 2104.

In block 2104, the computer system can annotate the training data with pressure data. As mentioned above, annotating and labeling the training data can be performed automatically by the computer system and/or manually by the relevant user. The training data can be annotated in 20 second windows to determine presence of snoring in the data. One or more other windows of time may also be used for annotating purposes. Once the bed systems are inclined, pressure signals can be detected by pressure sensors of the bed. These pressure signals can be used by the computer system (or the relevant user) to annotate the training data. As an example, if snore is detected in acoustic signals from a bed system that was inclined 15°, this training data can be annotated with pressure signals detected from this bed at the inclination of 15°. Annotating the training data with pressure signals from pressure sensors and/or load cells can beneficially train the model to accurately detect (i) snore from only acoustic signals during runtime use (since inclining the bed at various angles can muffle or otherwise make accurate detection of acoustic signals alone more challenging) and (ii) determine from which side of a bed the snore originates.

The computer system can train a machine learning model to detect snore using the annotated training data (block 2106). In some implementations, approximately 70% of the annotated training data can be used for training a DNN. 30% of the annotated training data can then be used for validation purposes. The DNN can be composed of convolutional layers, which extract spectral information, and recurrent layers, which keep track of time. Once the DNN is trained, the remaining 30% of the annotated training data may be used to determine model snore detection accuracy.

In some implementations, the computer system can generate classifiers from the annotated training data using artificial neural network machine learning techniques. The classifiers can then be trained using large sets of pre-classified variation patterns in acoustic signals (and/or pressure signals). For example, one bed or many beds may report the signals to a cloud reporting service for labeling, recording, and storing to then be used in training the classifiers.

One or more DNN trainers can be used to train the machine learning model (and/or the classifiers). As described above, a DNN can be generated, and the training data can be provided as input to the DNN to generate output. The output can be a classification (e.g., snore detected or no snore detected) and a confidence score. The confidence score can be a value on a scale of 0 to 1. As mentioned above, one or more other machine learning techniques can be used to train the machine learning model.

The computer system can then output the machine learning model for use during runtime (block 2108). Outputting the model can include storing the model in a data store, for later retrieval and/or use by the central controllers of different bed systems. Outputting the model can also include transmitting/deploying the model at the central controllers of the different bed systems.

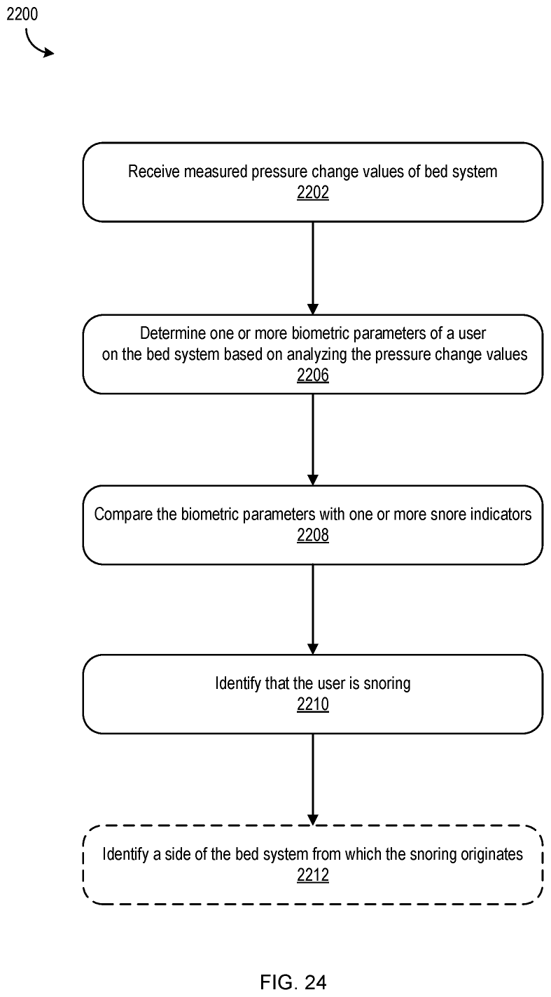

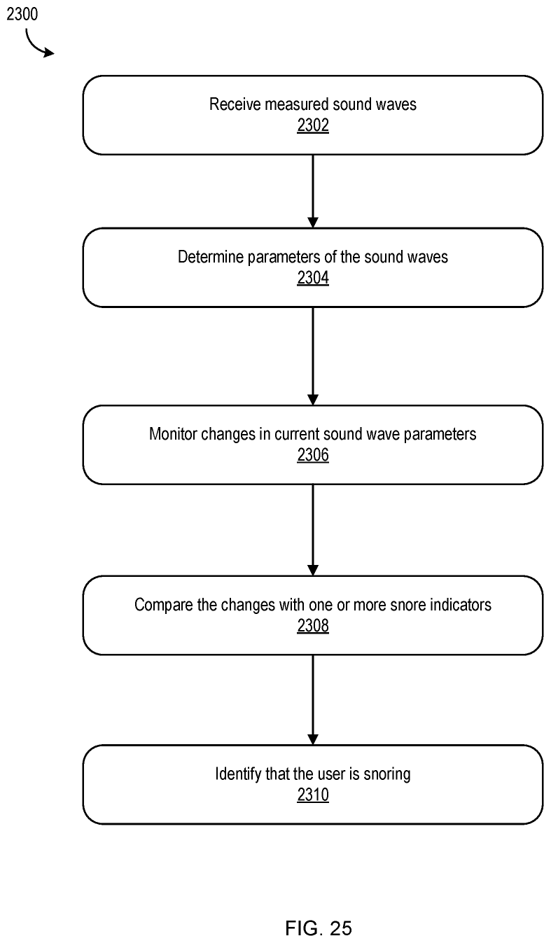

FIG. 24 is a flowchart of a process 2200 for determining that a user is snoring based on pressure data. In some implementations, as described herein and in the process 2200, snore detection can be determined based on pressure data. Sometimes, as described in reference to FIGS. 19 and 22, snore detection can be performed based on correlating pressure data with acoustic signals. Yet sometimes, as described in reference to FIGS. 22 and 25, snore detection can be performed based on acoustic signals.

The process 2200 can be performed by the central controller 1902. The process 2200 can also be performed by one or more other controllers, computing systems, devices, network of devices and/or systems, and/or cloud-based service/system. For illustrative purposes, the process 2200 is described from the perspective of a computer system.

Referring to the process 2200, the computer system can receive measured pressure change values of a bed system in block 2202. In some implementations, the computer system can execute instructions that cause one or more pressure sensors of the bed system to measure pressure variations. Other times, the pressure sensors can automatically detect and measure pressure variations and transmit those detected signals to the computer system. The signals can be transmitted in real-time. The signals can also be transmitted in near real-time or at one or more time intervals.

The computer system can determine one or more biometric parameters of a user on the bed system based on analyzing the pressure change values (block 2206). Through band-pass filtering of the pressure signal, BCG signals can be extracted. Such signals can include a breathing signal superposed with a cardiac signal of the user. Analysis of the BCG signals can be beneficial to estimate breathing rate and heartrate of the user.

For example, the computer system can process the pressure change values so that they can be used in further analysis by the bed system. Further analysis can include identification of peaks, valleys, or other aspects of the pressure change values that may indicate biometric parameters including but not limited to snore, heartrate, sleep apnea, and/or respiration rate.

The computer system can also compare the biometric parameters with one or more snore indicators in block 2208. Snore indicators can include predetermined values, ranges, and/or patterns that are indicative of snoring. Such indicators can be automatically identified by the computer system as indicative of snoring. Such indicators can also be identified by a relevant user as indicative of snoring. The indicators can be generated before run-time use and stored in a data store for retrieval and use during the process 2200.

In some implementations, comparing the biometric parameters with the snore indicators can be performed by a machine learning model. In other words, the computer system can provide the biometric parameters as input to a machine learning model, which can be trained to identify snore from the biometric parameters. The machine learning model can be trained as described in the process 2100 of FIG. 23. The machine learning model can output an indication of whether the biometric parameters are indicative of snoring and a confidence score associated with that indication.

Accordingly, the computer system can identify that the user is snoring in block 2210. If, for example, the biometric parameters satisfy values, ranges, and/or patterns indicative of snoring, the computer system can identify that the user is snoring. If, on the other hand, the biometric parameters do not satisfy the values, ranges, and/or patterns, then the computer system can determine that the user is not snoring. The user might have, for example, coughed, sneezed, or moved around on the bed, thereby causing pressure changes in the bed but not pressure changes indicative of snoring. After block 2210, the computer system can return to block 2202 and repeat the process 2200 until the user wakes up or for a predetermined amount of time. Other times, the process 2200 can stop after block 2210.

Optionally, the computer system can also identify a side of the bed system from which the snoring originates, as described throughout this disclosure (block 2212). For example, the computer system can receive a set of pressure signals from each side of the bed. The computer system can compare the sets of pressure signals to determine which set of pressure signals is indicative of the snoring. The computer system can correlate the set of pressure signals indicate of the snoring with a corresponding side of the bed. By identifying the side of the bed with the snore, the computer system can generate instructions to adjust that side to reduce or otherwise stop the snoring.

When identifying the side of the bed from which the snoring originates, the computer system can detect additional components in the pressure signals that result from a user snoring. Moreover, in some implementations, the computer system may also determine the side of the bed from which the snoring originates using the audio signals.

FIG. 25 is a flowchart of a process 2300 for determining that a user is snoring based on acoustic data. As described herein, snore can be detected based on acoustic data (e.g., signals). For example, a machine learning model can be trained and used to determine snore based on acoustic signals detected by one or more acoustic sensors (e.g., microphones) on, integrated into, and/or proximate a bed system.

The process 2300 can be performed by the central controller 1902. The process 2300 can also be performed by one or more other controllers, computing systems, devices, network of devices and/or systems, and/or cloud-based service/system. For illustrative purposes, the process 2300 is described from the perspective of a computer system.

Referring to the process 2300, the computer system can receive measured sound waves in block 2302. The computer system can execute instructions that cause acoustic sensors (e.g., microphones, voice controllers, etc.) of the bed system to measure sound waves (e.g., detect sound) near the bed system. In some implementations, the acoustic sensors may only measure sound waves that satisfy a predetermined decibel value and/or range. Doing so can preserve privacy of the user(s) in the bed system. Sometimes, the sound waves can be measured continuously or at predetermined sample rates. The sound waves can also be measured based on identifying frequency bands that do not contain intelligible human speech. For example, sound waves may only be measured if they have signals below 2 KHz.

In block 2304, the computer system can determine parameters of the sound waves. The sound waves can reflect breathing patterns. By estimating a breathing rate, which is typically lower during sleep than when the user is awake, the computer system can detect wake and sleep states of the user. As another example, the computer system can determine when the user is falling asleep based on the sound waves. The computer system can also determine whether the user is sleeping based on the sound waves. Moreover, the computer system can determine baseline parameters for the associated user, such as typical breathing patterns of the user (or breathing pattern of the user for the particular sleep cycle) and/or when the user typically falls asleep and/or wakes up.

The computer system can monitor changes in current sound wave parameters in block 2306. The computer system can monitor for changes (e.g., increases and/or decreases) in audible level, frequency, and/or wave patterns. Any of these changes can indicate that the user is snoring, waking up, and/or woken up. In other words, any of these changes can indicate that the user is deviating from their baseline parameters.

In block 2308, the computer system can compare the changes with one or more snore indicators. For example, if the computer system detects one or more changes with respect to the baseline parameters, the computer system can compare the current parameters with predetermined values/ranges of audible levels, frequencies, and wave patterns that are indicative of snoring. The predetermined values/ranges can be determined by the computer system (or another computing system) at another time and stored in a data store. The predetermined values/ranges can be retrieved by the computer system during runtime and used in block 2308.

The computer system can then identify that the user is snoring in block 2310. For example, if the changes in the current parameters satisfy one or more predetermined values/ranges, the computer system can determine that the user is snoring. After block 2310, the computer system can return to block 2302 and continuously monitor for changes in sound that is detected at the bed system. Sometimes, the process 2300 can stop. Sometimes, the process 2300 can be triggered at one or more predetermined time intervals and/or when a change in current parameters is detected/identified at the bed system.

One or more of the blocks described in the process 2300 can be performed using machine learning techniques. For example, instead of performing blocks 2308-2310, the computer system can provide measured sound waves as input to a machine learning model. The model can be trained using the techniques described in FIG. 23. The machine learning model can then determine whether the user is snoring based on analysis of the measured sound waves, as described throughout this disclosure.

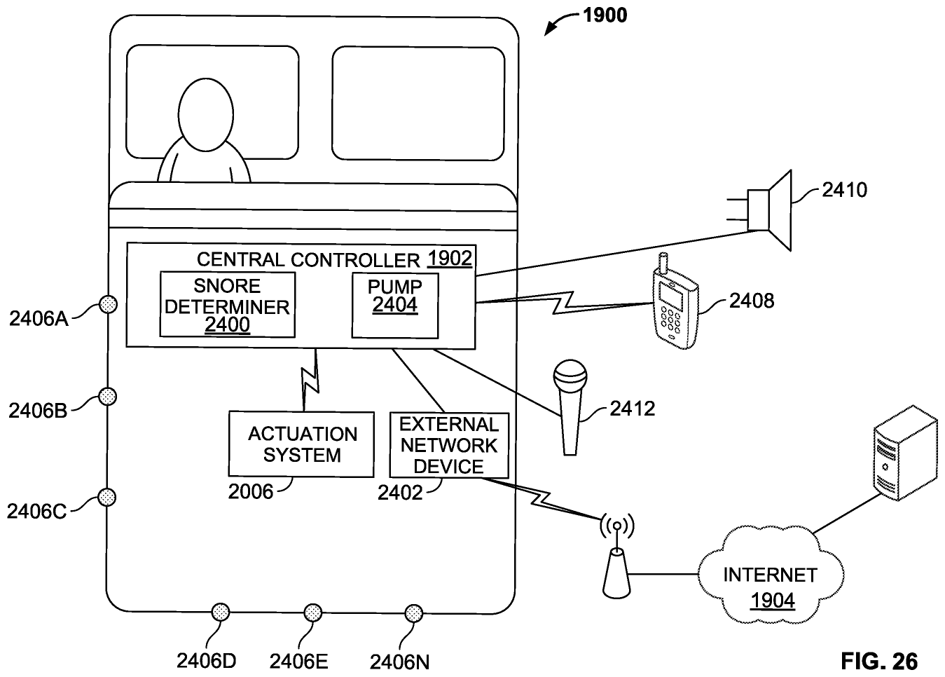

FIG. 26 is a conceptual diagram of a bed system 1900 for detecting and responding to snore. The bed system 1900 includes the central controller 1902 and the actuation system 2006 in communication with an external network device 2402, mobile device 2408 (e.g., or other type of remote controller) and a voice controller 2412. In addition to providing input for various types of commands, the mobile device 2408 can also be used for detecting snoring of a user. Thus, the mobile device 2408 can include any detection means capable of detecting sound waves (e.g., acoustic data/signals), such as a microphone. Similarly, in addition to providing for input of vocal commands, the voice controller 2412 can also be used for detecting snoring. Thus, the voice controller 2412 can include any detection means capable of detecting sound waves, such as a microphone. While described as an air bed, the bed system 1900 may also be other types of beds.

The bed system 1900 can be configured as a star topology with the central controller 1902 functioning as the hub and the actuation system 2006, the external network device 2402, the mobile device 2408, and the voice controller 2412 functioning as possible spokes, also referred to as components. Thus, in various examples, the central controller 1902 can act as a relay between the various components.

In some implementations, the central controller 1902 listens to communications (e.g., control signals) between components even if the communication is not being relayed through the central controller 1902. In other examples, different topologies may be used. For example, the components and the central controller 1902 may be configured as a mesh network in which each component may communicate with one or all of the other components directly, bypassing the central controller 1902. In various examples, a combination of topologies may be used. For example, the mobile device 2408 may communicate directly to the actuation system 2006 and/or a pump 2404 but also relay the communication to the central controller 1902.

The bed system 1900 can include the pump 2404, which can be controlled by the central controller 1902 (and/or a firmness controller) to regulate pressure in an air mattress of the bed system 1900. The pump 2404 can be part of the central controller 1902 in some implementations. Accordingly, the central controller 1902 may be responsible for pressure regulation as well as other functionality described herein. Moreover, although not depicted in FIG. 26, one or more pressure sensors can be integrated into, attached, or otherwise proximate the pump 2404. These pressure sensors can measure changes in pressure along/inside the pump 2404, which can be used by the central controller 1902 to determine whether the user is snoring and from which side of the bed the snoring originates.

In various examples, the actuation system 2006 can be configured to adjust a position of a bed mattress by adjusting a foundation of the bed system 1900. As described herein, the actuation system 2006 can activate one or more motors to tilt the foundation to a predetermined angle intended for reducing or otherwise stopping snore. In some implementations, the bed system 1900 can include a single foundation configured to adjust the position of a bed having a single mattress. In some implementations, the bed system 1900 can include two side-by-side foundations (or a foundation having two independently articulable sides) configured to adjust the position of a bed having a single mattress.

In various examples, additional controllers may communicate with the central controller 1902. These controllers may include, but are not limited to, audio/visual controllers for controlling one or more audio/visual components 2410 located near the bed system 1900. The central controller 1902 can therefore control power status and/or volume of the audio/visual components 2410. The audio/visual components 2410 can also include sound detection means, such as audio sensors and/or microphones. Therefore, the central controller 1902 can execute instructions that cause the audio/visual components 2410 to detect audio signals at the bed system 1900 that can be used by the central controller 1902 to determine whether the user is snoring.

The central controller 1902 can analyze data collected by sensors 2406A-N of the bed system 1900. The sensors 2406A-N, as described throughout this disclosure, can include acoustic sensors and/or pressure sensors. Using the data collected by the sensors 2406A-N, the central controller 1902 can determine whether the user is snoring and from which side of the bed the snoring originates. In particular, the central controller 1902 can include a snore determiner 2400. The snore determiner 2400 can be configured to perform the techniques described herein to detect whether the user is snoring. Based on a determination made by the snore determiner 2400, the central controller 1902 can generate instructions that, when executed by the actuation system 2006, cause the foundation of the bed system 1900 to be tilted (e.g., inclined) to a predetermined position, such as a position for snore mitigation.

Moreover, the central controller 1902 can determine biometric parameters about the user, such as heartrate, respiration rate, and/or movement, based on detected pressure signals. The biometric parameters can be used for determining whether the user is snoring and/or from which side of the bed the snoring originates. Thus, additional processing may be done using the collected data to determine various sleep data about the user. In some implementations, the data collected by the sensors 2406A-N can be sent to a cloud-based computing system for remote analysis via the network(s) 1904 (e.g., internet).

In various examples, the external network device 2402 includes a network interface to interact with an external server for processing and storage of data related to components in the bed system 1900. For example, the determined sleep data as described above may be transmitted via the network(s) 1904 from the central controller 1902 to the external network device 2402 for storage in a data store. In an example, acoustic and/or pressure signals detected by any of the sensors 2406A-N may be transmitted to the external server for additional analysis. The external network device 2402 may also analyze and filter the signals before transmitting them to the external server.

What is claimed is:

1. A bed system comprising:
a mattress;
an adjustable foundation; and
a controller comprising a processor, wherein the processor is configured to generate, in response to sensing snoring from a user of the bed system, instructions for tilting the adjustable foundation to a predetermined position such that a head portion of the adjustable foundation is higher than a foot portion of the adjustable foundation.

2. The bed system of claim 1, further comprising an acoustic sensor for sensing audio at the bed system,
wherein the processor is further configured to:
receive acoustic signals from the acoustic sensor; and
analyze the acoustic signals to determine that the user is snoring.

3. The bed system of claim 1, further comprising a pressure sensor for sensing pressure on the mattress,
wherein the processor is further configured to:
receive pressure signals from the pressure sensor; and
analyze the pressure signals to determine that the user is snoring.

4. The bed system of claim 3, wherein the processor is further configured to analyze the pressure signals to determine a left side or a right side of the mattress from which the snoring originates.

5. The bed system of claim 1, further comprising a plurality of pressure sensors, wherein each of the plurality of pressure sensors are positioned in a support leg of the adjustable foundation, wherein the plurality of pressure sensors are configured to sense pressure on the bed system indicative of a location of the user on the mattress.

6. The bed system of claim 1, further comprising at least one sensor,
wherein the processor is further configured to:
receive signals from the at least one sensor; and
analyze the signals to determine that the user is snoring.

7. The bed system of claim 6, wherein the at least one sensor is at least one of a pressure sensor and an acoustic sensor.

8. The bed system of claim 6, wherein the signals include at least one of acoustic signals and pressure signals.

9. The bed system of claim 6, wherein the signals include acoustic signals and pressure signals,
wherein the processor is further configured to correlate the acoustic signals with the pressure signals to determine (i) whether the user is snoring and (ii) a left side or right side of the mattress from which the snoring originates.

10. The bed system of claim 1, further comprising an articulation system configured to tilt the adjustable foundation to the predetermined position.

11. The bed system of claim 10, wherein:
the processor is configured to transmit, in response to determining that the user is snoring, instructions to the articulation system to tilt the adjustable foundation to the predetermined position; and
the articulation system is configured to:
raise, in response to receiving the instructions from the processor, legs supporting the head portion of the adjustable foundation to a height that corresponds to the angle between the head portion of the adjustable foundation and the ground when the adjustable foundation is in the predetermined position.

12. The bed system of claim 11, wherein the articulation system is configured to lower, in response to receiving the instructions from the processor, legs supporting the foot portion of the adjustable foundation to a height that corresponds to the angle between the foot portion of the adjustable foundation and the ground when the adjustable foundation is in the predetermined position.

13. The bed system of claim 12, wherein the angle between the foot portion of the adjustable foundation and the ground is less than 7°.

14. The bed system of claim 11, wherein:
the processor is configured to transmit, in response to determining that the user has woken up, instructions to the articulation system to move the adjustable foundation into a neutral position; and
the articulation system is configured to lower, in response to receiving the instructions from the processor, the legs supporting the head portion of the adjustable foundation to a second height that corresponds to the neutral position, wherein in the neutral position, the adjustable foundation is parallel with the ground.

15. The bed system of claim 14, wherein the articulation system is configured to raise, in response to receiving the instructions from the processor, the legs supporting the foot portion of the adjustable foundation to the second height that corresponds to the neutral position.

16. The bed system of claim 11, wherein the articulation system is configured to adjust, based on receiving an indication of user input from a user device indicating selection of an option to move the adjustable foundation to another position, a height of one or more of the legs supporting the adjustable foundation to move the adjustable foundation to the another position.

17. The bed system of claim 1, wherein in the predetermined position:
the head portion of the adjustable foundation is tilted above the foot portion of the adjustable foundation, and
the adjustable foundation remains inclined along a substantially straight plane.

18. The bed system of claim 1, wherein in the predetermined position, the adjustable foundation is maintained along an inclined and substantially straight plane.

19. A bed system comprising:
a mattress;
an adjustable foundation; and
a controller comprising a processor, wherein the processor is configured to generate, in response to sensing snoring from a user of the bed system, instructions for tilting the adjustable foundation to a predetermined position such that a head portion of the adjustable foundation and a foot portion of the adjustable foundation are in-line with respect to each-other and tilted with respect to horizontal.

20. A bed system comprising:
a mattress;
an adjustable foundation; and
a controller comprising a processor, wherein the processor is configured to generate, in response to sensing snoring from a user of the bed system, instructions for tilting a head portion of the adjustable foundation and a foot portion of the adjustable foundation at substantially a same angle.
